(19) United States
(12) Patent Application Publication
(10) Pub. No.: US 2024/0110214 A1
(43) Pub. Date: Apr. 4, 2024

(54) RNA MANUFACTURING

(71) Applicant: BioNTech SE, Mainz (DE)

(72) Inventors: Thomas Ziegenhals, Nierstein (DE); Andreas Kuhn, Mainz (DE); Stephanie Fesser, Mannheim (DE)

(21) Appl. No.: 18/266,135

(22) PCT Filed: Dec. 7, 2021
(86) PCT No.: PCT/EP21/84488

§ 371 Date: Jun. 8, 2023

Related U.S. Application Data
(60) Provisional application No. 63/132,473, filed on Dec. 30, 2020.
(60) Provisional application No. 63/123,465, filed on Dec. 9, 2020.

(51) Int. Cl. C12P 19/34; A61K 31/7115

(52) U.S. Cl. CPC C12P 19/34; A61K 31/7115

(57) ABSTRACT

The present disclosure provides technologies for performing in vitro transcription that can generate product RNA preparations with reduced levels of certain contaminants (e.g., aberrant products), and particularly of double-stranded RNA (dsRNA).

### TECHNICAL FIELD OF THE INVENTION

The present invention relates to methods for dsRNA reduction during in vitro transcription through step wise addition of nucleotides. The present invention further relates to nucleic acids produced by a method of the invention and to the use of such nucleic acids in methods of treating a subject in need thereof.

### BACKGROUND

In vitro production of RNA has become increasingly important in the biotechnology and pharmaceutical industries. Improvements to manufacturing processes, particularly those that can produce high-quality RNA (e.g., mRNA) at large scale, including specifically therapeutic-grade RNA (e.g., mRNA), are important and valuable.

### SUMMARY

RNA transcribed in vitro (e.g., by T7 RNA Polymerase (RNAP)) can contain aberrant products, including at significant levels. Without wishing to be bound by theory, it is proposed that some or all such products can be generated by unconventional activity of a utilized RNAP. One such aberrant product is dsRNA, which can prove particularly problematic, for example in light of its propensity to induce inflammatory cytokines and/or to activate immuno-effector proteins which, among other things, can lead to inhibition of protein synthesis. dsRNA is typically the major contaminant of in vitro RNA transcription reactions.

Commonly, aberrant products, and particularly dsRNA, are removed from in vitro-transcribed RNA preparations by purification; a variety of purification technologies are available (e.g., via LiCl and/or alcohol-based precipitation, size exclusion and/or ion-exchange chromatography, silica matrix purification, ion-pair reversed-phase high performance liquid chromatography [HPLC], cellulose-based separation, etc.). However, most or all of such purification strategies may be impractical and/or otherwise not ideal, particularly for commercial scale and/or pharmaceutical-grade production, among other things because they often remove the desired RNA product together with the aberrant product (and/or other contaminant), resulting in undesirably high loss of RNA product.

Among other things, the present disclosure provides an insight that, surprisingly, limiting the amount of UTP or a functional analog thereof when synthesizing RNA by transcription reactions, and supplementing the reaction mix with UTP or a functional analog thereof during the course of the transcription reaction, yields RNA of increased purity, reduced immunogenicity and favorable stability.

Furthermore, the present disclosure provides an insight that benefits could be gained by reducing production of aberrant products (and/or other contaminants) in the first instance. The present disclosure provides technologies for performing in vitro transcription that can generate product RNA preparations with reduced levels of certain contaminants (e.g., aberrant products), and particularly of dsRNA. Advantages of provided technologies include, but are not limited to, more efficient manufacturing, including higher yield of product RNA (e.g., less product loss during processing), fewer processing steps (which may contribute to reduced product loss), lower production cost, shorter production timelines, etc. Moreover, the present disclosure teaches that provided improved production technologies (e.g., improved transcription reaction conditions) have various advantages (including the foregoing) even relative to improved purification technologies.

In one aspect, the invention relates to a method of producing an RNA comprising transcribing RNA from a DNA template using a reaction mix which comprises adenosine triphosphate (ATP), guanosine triphosphate (GTP), cytidine triphosphate (CTP) and uridine triphosphate (UTP), or functional analogs thereof, wherein the starting concentration of UTP, or a functional analog thereof, is lower than the starting concentration of CTP and/or ATP, or a functional analog thereof, wherein the method comprises supplementing the reaction mix during the course of the transcription reaction with a composition which comprises UTP, or a functional analog thereof, and is substantially free of CTP or ATP, or a functional analog thereof.

In one aspect, the invention relates to a method of producing an RNA comprising transcribing RNA from a DNA template using a reaction mix which comprises adenosine triphosphate (ATP), guanosine triphosphate (GTP), cytidine triphosphate (CTP) and uridine triphosphate (UTP), or functional analogs thereof, wherein the starting concentration of CTP, or a functional analog thereof, is equal to the starting concentration of ATP, or a functional analog thereof, and wherein the starting concentration of UTP, or a functional analog thereof, is lower than the starting concentration of CTP or ATP, or a functional analog thereof, wherein the method comprises supplementing the reaction mix with UTP, or a functional analog thereof, during the course of the transcription reaction.

In one aspect, the invention relates to a method of producing a composition comprising RNA having a reduced double-stranded (ds) RNA content, wherein the method comprises transcribing RNA from a DNA template using a reaction mix which comprises adenosine triphosphate (ATP), guanosine triphosphate (GTP), cytidine triphosphate (CTP) and uridine triphosphate (UTP), or functional analogs thereof, wherein the starting concentration of UTP, or a functional analog thereof, is lower than the starting concentration of CTP and/or ATP, or a functional analog thereof, wherein the method comprises supplementing the reaction mix during the course of the transcription reaction with a composition which comprises UTP, or a functional analog thereof, and is substantially free of CTP or ATP, or a functional analog thereof.

In one aspect, the invention relates to a method of producing a composition comprising RNA having a reduced double-stranded (ds) RNA content, wherein the method comprises transcribing RNA from a DNA template using a reaction mix which comprises adenosine triphosphate (ATP), guanosine triphosphate (GTP), cytidine triphosphate (CTP) and uridine triphosphate (UTP), or functional analogs thereof, wherein the starting concentration of CTP, or a functional analog thereof, is equal to the starting concentration of ATP, or a functional analog thereof, and wherein the starting concentration of UTP, or a functional analog thereof, is lower than the starting concentration of CTP or ATP, or a functional analog thereof, wherein the method comprises supplementing the reaction mix with UTP, or a functional analog thereof, during the course of the transcription reaction.

In some embodiments, the double-stranded (ds) RNA content of the composition comprising RNA is reduced compared to the dsRNA content of a composition comprising RNA transcribed from the same DNA template using equimolar amounts of adenosine triphosphate (ATP), guanosine triphosphate (GTP), cytidine triphosphate (CTP) and uridine triphosphate (UTP), or functional analogs thereof.

In some embodiments, the immunogenicity of the composition comprising RNA is reduced compared to the immunogenicity of a composition comprising RNA transcribed from the same DNA template using equimolar amounts of adenosine triphosphate (ATP), guanosine triphosphate (GTP), cytidine triphosphate (CTP) and uridine triphosphate (UTP), or functional analogs thereof.

In some embodiments, uridine triphosphate (UTP), or a functional analog thereof, is present in a starting concentration that limits the rate of transcription.

In some embodiments, the ratio of the starting concentration of uridine triphosphate (UTP), or a functional analog thereof, to the starting concentration of cytidine triphosphate (CTP) or adenosine triphosphate (ATP), or a functional analog thereof, is between about 1:1.5 and about 1:15.

In some embodiments, the reaction mix is supplemented with uridine triphosphate (UTP), or a functional analog thereof, when the concentration of UTP, or a functional analog thereof, nears depletion.

In some embodiments, the reaction mix is supplemented at least once with uridine triphosphate (UTP), or a functional analog thereof, during the course of the transcription reaction.

In some embodiments, the reaction mix is supplemented continuously with uridine triphosphate (UTP), or a functional analog thereof, during the course of the transcription reaction.

In some embodiments, the reaction mix is supplemented periodically with uridine triphosphate (UTP), or a functional analog thereof, during the course of the transcription reaction.

In some embodiments, supplementing the reaction mix with uridine triphosphate (UTP), or a functional analog thereof, maintains or restores the initial ratio of the concentration of UTP, or a functional analog thereof, to the concentration of cytidine triphosphate (CTP) or adenosine triphosphate (ATP), or a functional analog thereof.

In some embodiments, the reaction mix is supplemented with uridine triphosphate (UTP), or a functional analog thereof, until the end of the transcription reaction.

In some embodiments, the starting concentration of guanosine triphosphate (GTP), or a functional analog thereof, is lower than the starting concentration of cytidine triphosphate (CTP) or adenosine triphosphate (ATP), or a functional analog thereof. In some embodiments, guanosine triphosphate (GTP), or a functional analog thereof, preferably is present in a starting concentration that limits the rate of transcription.

In some embodiments, the ratio of the starting concentration of guanosine triphosphate (GTP), or a functional analog thereof, to the starting concentration of cytidine triphosphate (CTP) or adenosine triphosphate (ATP), or a functional analog thereof, is between about 1:1.5 and about 1:15.

In some embodiments, the reaction mix is supplemented with guanosine triphosphate (GTP), or a functional analog thereof, during the course of the transcription reaction.

In some embodiments, the reaction mix is supplemented with guanosine triphosphate (GTP), or a functional analog thereof, when the concentration of GTP, or a functional analog thereof, nears depletion.

In some embodiments, the reaction mix is supplemented at least once with guanosine triphosphate (GTP), or a functional analog thereof, during the course of the transcription reaction.

In some embodiments, the reaction mix is supplemented continuously with guanosine triphosphate (GTP), or a functional analog thereof, during the course of the transcription reaction.

In some embodiments, the reaction mix is supplemented periodically with guanosine triphosphate (GTP), or a functional analog thereof, during the course of the transcription reaction.

In some embodiments, supplementing the reaction mix with guanosine triphosphate (GTP), or a functional analog thereof, maintains or restores the initial ratio of the concentration of GTP, or a functional analog thereof, to the concentration of cytidine triphosphate (CTP) or adenosine triphosphate (ATP), or a functional analog thereof.

In some embodiments, the reaction mix is supplemented with guanosine triphosphate (GTP), or a functional analog thereof, until the end of the transcription reaction.

In some embodiments, a provided method does not comprise supplementing the transcription mix with cytidine triphosphate (CTP) and/or adenosine triphosphate (ATP), or a functional analog thereof, during the course of the transcription reaction.

In some embodiments, the reaction mix comprises a start nucleotide corresponding to the first nucleotide in the RNA molecule.

In some embodiments, the start nucleotide is a nucleoside monophosphate, a nucleoside diphosphate, a nucleoside triphosphate or a dinucleoside triphosphate.

In some embodiments, the start nucleotide is a 5′ cap or a 5′ cap analog.

In some embodiments, the 5′ cap or 5′ cap analog is selected from the group consisting of G[5′]ppp[5′]G, m7G[5′]ppp[5′]G, m₃^{2,2,7}G[5′]ppp[5′]G, m₂^{7,3′-O}G[5′]ppp[5′]G (3′-ARCA), m₂^{7,2′-O}GpppG (2′-ARCA), m₂^{7,2′-O}GppspG D1 (β-S-ARCA D1), m₂^{7,2′-O}GppspG D2 (β-S-ARCA D2) and m₂^{7,3′-O}Gppp(m₂′-O)ApG (CC413).

In some embodiments, the 5′ cap or 5′ cap analog in the reaction mix is present in excess compared to guanosine triphosphate (GTP), or a functional analog thereof.

In some embodiments, the ratio of the starting concentration of 5′ cap or 5′ cap analog to the starting concentration of guanosine triphosphate (GTP), or a functional analog thereof, is between about 2:1 and about 20:1.

In some embodiments, the ratio of the starting concentration of 5′ cap or 5′ cap analog to the starting concentration of guanosine triphosphate (GTP), or a functional analog thereof, is about 4:1.

In some embodiments, the reaction mix further comprises an RNA polymerase, a buffer and at least one monovalent or divalent cation.

In some embodiments, the cation is Li⁺, Na⁺, K⁺, NH⁴⁺, tris(hydroxymethyl)aminomethane cation, Mg²⁺, Ba²⁺ or Mn²⁺.

In some embodiments, the RNA polymerase is selected from the group consisting of T7 RNA polymerase, T3 RNA polymerase and SP6 RNA polymerase.

In some embodiments, the functional analog of uridine triphosphate (UTP) is selected from the group consisting of Pseudo-UTP, N1-Methylpseudo-UTP, 2-Thio-UTP and 4-Thio-UTP.

In some embodiments, the functional analog of guanosine triphosphate (GTP) is selected from the group consisting of 7-Deaza-GTP, N1-Methyl-GTP and O6-Methyl-GTP.

In some embodiments, the DNA template encodes one or more of a 5′ untranslated region (UTR), a 3′ UTR, an open reading frame and a poly(A)-tail.

In some embodiments, the RNA comprises one or more of a 5′ untranslated region (UTR), a 3′ UTR, an open reading frame and a poly(A)-tail.

In some embodiments, the RNA encodes at least one peptide or protein.

In some embodiments, the RNA is mRNA.

In some embodiments, the RNA is a self-replicating RNA.

In some embodiments, RNA produced by a method of the invention has a length of between 200 to 20000 nucleotides, between 200 to 12000 nucleotides, between 200 to 8000 nucleotides, between 500 to 5000 nucleotides, between 500 to 2500 nucleotides, in particular between 600 to 2500 nucleotides or between 800 to 2000 nucleotides.

In some embodiments, the pH value of the reaction mix is kept substantially constant during the course of the transcription reaction.

In some embodiments, the progress of the transcription reaction is monitored in real time.

In some embodiments, the method is performed using a bioreactor.

In one aspect, the invention relates to an RNA produced by a method of the invention.

In one aspect, the invention relates to a composition comprising RNA produced by the method of the invention.

In one aspect, the invention relates to a method of treating a subject comprising the steps of: (i) obtaining RNA produced by the method of the invention, or obtaining a composition comprising RNA produced by the method of the invention, and (ii) administering the RNA or the composition comprising RNA to the subject.

In one aspect, the invention relates to a method of producing an RNA by in vitro transcription, wherein the method comprises restricting concentration of UTP or functional analogs thereof during the in vitro transcription reaction.

In one aspect, the invention relates to an in vitro transcription reaction comprising an RNA template comprising a promoter that directs transcription of a template to generate a transcript, optionally with a polyA sequence element; an RNA polymerase that acts on the promoter; and adenosine triphosphate (ATP), guanosine triphosphate (GTP), cytidine triphosphate (CTP) and uridine triphosphate (UTP), or functional analogs thereof, wherein the starting concentration of UTP or functional analog thereof, is lower than the concentration of CTP and/or ATP or functional analogs thereof.

In one aspect, the invention relates to a method of treating a subject by administering the RNA produced by a method of the invention or the composition comprising RNA produced by a method of the invention to the subject.

In one aspect, the invention relates to a method for reducing the amount of double-stranded RNA resulting from in vitro transcription of RNA, the method comprising in vitro transcribing RNA from a template under transcription conditions in which the amount or concentration of UTP or a functional analog thereof is a limiting amount or concentration for the transcribing of the RNA in comparison to the amount or concentration of one or more of ATP, CTP and/or GTP or a respective analog thereof.

In one aspect, the invention relates to a method for reducing the amount of double-stranded RNA in a composition comprising RNA resulting from in vitro transcription of RNA, the method comprising in vitro transcribing RNA from a template under transcription conditions in which the amount or concentration of UTP or a functional analog thereof is a limiting amount or concentration for the transcribing of the RNA in comparison to the amount or concentration of one or more of ATP, CTP and/or GTP or a respective analog thereof.

In one aspect, the invention relates to a method for reducing the immunogenicity of a composition comprising RNA resulting from in vitro transcription of RNA, the method comprising in vitro transcribing RNA from a template under transcription conditions in which the amount or concentration of UTP or a functional analog thereof is a limiting amount or concentration for the transcribing of the RNA in comparison to the amount or concentration of one or more of ATP, CTP and/or GTP or a respective analog thereof.

In an embodiment, the double-stranded RNA is the result of at least two distinct RNA molecules annealing to each other, i.e., the result of inter-molecular binding. In an embodiment, the double-stranded RNA is the result of intra-molecular binding, i.e., parts of an RNA molecule annealing to itself, e.g., in cases where the transcript backfolds on itself.

### BRIEF DESCRIPTION OF THE DRAWING

FIG. 1 demonstrates exemplary results for dsRNA content generated by an IVT reaction transcribing unmodified RNA with step-wise addition of NTP. RNA was in vitro transcribed with reduced starting concentration (limited) of indicated NTP. G-GTP, A-ATP, U-UTP, A/U-ATP+UTP. The limited NTP were fed step-wise to the IVT reaction until the final concentration NTP was reached for all NTPs. All RNA were co transcriptionally capped using CC413 cap analog. As a control the limitation of GTP was used. A. RNA yield was unaffected compared to control by the type of NTP that was fed over the course of the reaction. B. RNA integrity was unaffected compared to control by the type of NTP that was fed over the course of the reaction. C. dsRNA content was increased compared to control when ATP was fed and reduced when UTP was fed. Feeding both ATP and UTP abrogated each other's effect, resulting in a dsRNA content comparable to that of control (GTP fed). D. Capping efficiency was reduced compared to control when GTP was not fed.

FIG. 2 demonstrates exemplary results for dsRNA content generated by an IVT reaction of unmodified RNA with step-wise addition of UTP or GTP and UTP, which rescues capping efficiency. RNA was in vitro transcribed with reduced starting concentration (limited) of indicated NTP. G-GTP, U-UTP, G/U-GTP+UTP. The limited NTP were step wise fed to the IVT reaction until the final concentration NTP was reached for all NTPs. As a control the limitation of GTP was used. All RNA were co transcriptionally capped using D1-β-S1 ARCA cap analog. A. RNA yield was increased compared to control when UTP or UTP and GTP in combination were fed under these reaction conditions. B. Integrity of purified RNA was reduced when UTP or GTP and UTP were fed. When GTP and UTP were fed in combination RNA integrity was rescued to the level of that when the IVT reaction was GTP fed (control). C. dsRNA content was reduced by feeding UTP compared to the control GTP fed. Feeding both GTP und UTP reduces the dsRNA content as well, but to a lesser extent than the UTP fed alone. D. Capping efficiency was reduced compared to control when feeding UTP alone but was rescued by feeding GTP and UTP in combination.

FIG. 3 demonstrates exemplary results for dsRNA content generated by IVT reaction transcribing N1-methyl psueoduridine (m1ΨTP) containing RNA with step-wise addition of m1ΨTP or m1ψTP and GTP. RNA was in vitro transcribed with reduced starting concentration (limited) of indicated NTP. G-GTP, m1Ψ-m1ΨTP, G/m1Ψ-GTP+m1ΨTP. The limited NTP were fed step wise to the IVT reaction until the final concentration NTP was reached for all NTPs. As a control the limitation of GTP was used. All RNA were co transcriptionally capped using CC413 GAG cap analog. A. RNA yield was unaffected compared to control by the type of NTP that was fed over the course of the reaction. B. RNA Integrity was reduced compared to control when m1ΨTP was fed. When GTP and m1YTP were fed in combination, RNA integrity was rescued to the level of the GTP fed control. C. dsRNA content was reduced by feeding m1ΨTP compared to the standard GTP fed control. Feeding of both GTP und m1ΨTP reduced dsRNA content comparably to those from a m1ΨTP-only fed reaction. D. Capping efficiency was reduced compared to control when m1ΨTP fed, but were rescued by feeding both GTP and m1ΨTP.

FIG. 4 demonstrates exemplary results of higher order structure for RNA product using circular dichroism.

### CERTAIN DEFINITIONS

Certain terms used herein may be understood to be defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

Also, indications of relative amounts of a component characterized by a generic term are typically meant to refer to the total amount of all specific variants or members covered by said generic term. If a certain component defined by a generic term is specified to be present in a certain relative amount, and if this component is further characterized to be a specific variant or member covered by the generic term, it is meant that no other variants or members covered by the generic term are additionally present such that the total relative amount of components covered by the generic term exceeds the specified relative amount; more preferably no other variants or members covered by the generic term are present at all.

A: Terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

About or Approximately: The term “about” or “approximately”, when used herein in reference to a value, refers to a value that is similar, in context to a stated reference value. In general, those skilled in the art, familiar with the context, will appreciate the relevant degree of variance encompassed by “about” or “approximately” in that context. Those skilled in the art will appreciate that, in many embodiments (as will be understood from context), the term “about” means approximately or nearly, and in the context of a numerical value or range set forth herein preferably means +/−10% of the numerical value or range. For example, in some embodiments, the term “about” or “approximately” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, intradermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may be intramuscular. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Agent: In general, the term “agent”, as used herein, is used to refer to an entity (e.g., for example, a lipid, metal, nucleic acid, polypeptide, polysaccharide, small molecule, etc., or complex, combination, mixture or system [e.g., cell, tissue, organism] thereof), or phenomenon (e.g., heat, electric current or field, magnetic force or field, etc.). In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc., as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with the present disclosure is in a format selected from, but not limited to, intact IgA, IgG, IgE or IgM antibodies; bi- or multi-specific antibodies (e.g., Zybodies®, etc.); antibody fragments such as Fab fragments, Fab′ fragments, F(ab′)2 fragments, Fd′ fragments, Fd fragments, and isolated complementarity determining regions (CDRs) or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments, an included CDR is substantially identical to a reference CDR in that it shows at least 95%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an included CDR is substantially identical to a reference CDR in that at least one amino acid within the included CDR is substituted as compared with the reference CDR but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Antibody agents can be made by the skilled person using methods and commercially available services and kits known in the art. For example, methods of preparation of monoclonal antibodies are well known in the art and include hybridoma technology and phage display technology. Further antibodies suitable for use in the present disclosure are described, for example, in the following publications: Antibodies A Laboratory Manual, Second edition. Edward A. Greenfield. Cold Spring Harbor Laboratory Press (Sep. 30, 2013); Making and Using Antibodies: A Practical Handbook, Second Edition. Eds. Gary C. Howard and Matthew R. Kaser. CRC Press (Jul. 29, 2013); Antibody Engineering: Methods and Protocols, Second Edition (Methods in Molecular Biology). Patrick Chames. Humana Press (Aug. 21, 2012); Monoclonal Antibodies: Methods and Protocols (Methods in Molecular Biology). Eds. Vincent Ossipow and Nicolas Fischer. Humana Press (Feb. 12, 2014); and Human Monoclonal Antibodies: Methods and Protocols (Methods in Molecular Biology). Michael Steinitz. Humana Press (Sep. 30, 2013)).

Antibodies may be produced by standard techniques, for example by immunization with the appropriate polypeptide or portion(s) thereof, or by using a phage display library. If polyclonal antibodies are desired, a selected mammal (e.g., mouse, rabbit, goat, horse, etc.) is immunized with an immunogenic polypeptide bearing a desired epitope(s), optionally haptenized to another polypeptide. Depending on the host species, various adjuvants may be used to increase immunological response. Such adjuvants include, but are not limited to, Freund's, mineral gels such as aluminum hydroxide, and surface-active substances such as lysolecithin, pluronic polyols, polyanions, peptides, oil emulsions, keyhole limpet hemocyanin, and dinitrophenol. Serum from the immunized animal is collected and treated according to known procedures. If serum containing polyclonal antibodies to the desired epitope contains antibodies to other antigens, the polyclonal antibodies can be purified by immunoaffinity chromatography or any other method known in the art. Techniques for producing and processing polyclonal antisera are well known in the art.

Antigen: The term “antigen”, as used herein, refers to an agent that elicits an immune response; and/or (ii) an agent that binds to a T cell receptor (e.g., when presented by an MHC molecule) or to an antibody. In some embodiments, an antigen elicits a humoral response (e.g., including production of antigen-specific antibodies); in some embodiments, an antigen elicits a cellular response (e.g., involving T-cells whose receptors specifically interact with the antigen). In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer [e.g., other than a nucleic acid or amino acid polymer) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen.

Autologous: The term “autologous” is used to describe anything that is derived from the same subject. For example, “autologous cell” refers to a cell derived from the same subject. Introduction of autologous cells into a subject is advantageous because these cells overcome the immunological barrier which otherwise results in rejection.

Allogeneic: The term “allogeneic” is used to describe anything that is derived from different individuals of the same species. Two or more individuals are said to be allogeneic to one another when the genes at one or more loci are not identical.

Base pair: As is understood in the art, a “base pair” is a structural motif of a secondary structure wherein two nucleotide bases associate with each other through hydrogen bonds between donor and acceptor sites on the bases. Complementary bases, A:U and G:C are understood to be able to form stable base pairs through hydrogen bonds between donor and acceptor sites on the bases; the A:U and G:C base pairs are called Watson-Crick base pairs. A weaker base pair (called Wobble base pair) is formed by the bases G and U (G:U). Base pairs A:U and G:C may be referred to as “canonical” base pairs. Other base pairs, such as G:U (which occurs fairly often in RNA) and other relatively uncommon base-pairs (e.g. A:C; U:U) may be referred to as non-canonical base pairs.

Batch: As used herein, the term “batch” or “batch reaction” or similar terms refers to at least one discrete supplementation event for at least one component (e.g., in some embodiments) specifically at least one for UTP or analog thereof, optionally at least one for other component(s), optionally multiple components supplemented in same discrete supplementation event.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

Bioreactor: The term “bioreactor” as used herein refers to a vessel used for in vitro transcription described herein. A bioreactor can be of any size so long as it is useful for in vitro transcription. For example, in some embodiments, a bioreactor can be at least 0.5 liter, including 1, 2, 3, 4, 5, 6, 7, 8, 9, 10, 15, 20, 25, 30, 35, 40, 45, 50 liters or more, or any volume in between. The internal conditions of the bioreactor, including, but not limited to pH and temperature, are typically controlled during in vitro transcription. The bioreactor can be composed of any material that is suitable for in vitro transcription under the conditions as described herein, including glass, plastic or metal. One of ordinary skill in the art will be aware of and will be able to choose suitable bioreactor volume for use in practicing in vitro transcription.

Cap: As used herein, the term “cap” refers to a structure comprising or essentially consisting of a nucleoside-5′-triphosphate that is typically joined to a 5′-end of an uncapped RNA (e.g., an uncapped RNA having a 5′-diphosphate). In some embodiments, a cap is or comprises a guanine nucleotide. In some embodiments, a cap is or comprises a naturally-occurring RNA 5′ cap, including, e.g., but not limited to a N7-methylguanosine cap, which has a structure designated as “m7G.” In some embodiments, a cap is or comprises a synthetic cap analog that resembles an RNA cap structure and possesses the ability to stabilize RNA if attached thereto, including, e.g., but not limited to anti-reverse cap analogs (ARCAs) known in the art). Those skilled in the art will appreciate that methods for joining a cap to a 5′ end of an RNA are known in the art. For example, in some embodiments, a capped RNA may be obtained by in vitro capping of RNA that has a 5′ triphosphate group or RNA that has a 5′ diphosphate group with a capping enzyme system (including, e.g., but not limited to vaccinia capping enzyme system or Saccharomyces cerevisiae capping enzyme system). Alternatively, a capped RNA can be obtained by in vitro transcription (IVT) of a DNA template, wherein, in addition to the GTP, an IVT system also contains a cap analog, e.g., as known in the art. Non-limiting examples of a cap analog include a m7GpppG cap analog or an N7-methyl-, 2′-O-methyl-GpppG ARCA cap analog or an N7-methyl-, 3′-O-methyl-GpppG ARCA cap analog, or any commercially available cap analogs, including, e.g., CleanCap (Trilink), EZ Cap, etc. In some embodiments, a cap analog is or comprises a trinucleotide cap analog. Various cap analogs are described herein and known in the art, e.g., commercially available.

Codon: As is understood in the art, the term “codon” refers to a base triplet in a coding nucleic acid that specifies which amino acid will be added next during protein synthesis at the ribosome.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Complementary: As used herein, the term “complementary” is used in reference to oligonucleotide hybridization related by base-pairing rules. For example, the sequence “C-A-G-T” is complementary to the sequence “G-T-C-A.” Complementarity can be partial or total. Thus, any degree of partial complementarity is intended to be included within the scope of the term “complementary” provided that the partial complementarity permits oligonucleotide hybridization. Partial complementarity is where one or more nucleic acid bases is not matched according to the base pairing rules. Total or complete complementarity between nucleic acids is where each and every nucleic acid base is matched with another base under the base pairing rules. As is understood in the art, percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” or “fully complementary” means that all the contiguous residues of a nucleic acid sequence will hydrogen bond with the same number of contiguous residues in a second nucleic acid sequence. In many embodiments, degree of complementarity according to the invention is at least 70%, preferably at least 75%, preferably at least 80%, more preferably at least 85%, even more preferably at least 90% or most preferably at least 95%, 96%, 97%, 98% or 99%. In certain embodiments, the degree of complementarity according to the invention is 100%.

Comprising: Unless expressly specified otherwise, the term “comprising” is used in the context of the present document to indicate that further members may optionally be present in addition to the members of the list introduced by “comprising”. It is, however, contemplated as a specific embodiment of the present invention that the term “comprising” encompasses the possibility of no further members being present, i.e. for the purpose of this embodiment “comprising” is to be understood as having the meaning of “consisting of”

Decreasing, reducing, inhibiting: As is understood in the art, terms such as “decreasing”, “reducing” or “inhibiting” may be used herein to refer to an ability to cause an overall and/or relative decrease, e.g., in an entity, event, frequency, activity, etc. In some embodiments, a decrease, for example, may be 5% or greater, 10% or greater, 20% or greater, 25% or greater, 30% or greater, 35% or greater, 40% or greater, 45% or greater, in some embodiments of 50% or greater, 60% or greater, 70% or greater, and in some embodiments 75% or greater. In some embodiments, a decrease may be 2 fold or greater, 3 fold or greater, 4 fold or greater, 5 fold or greater, 6 fold or greater, 7 fold or greater, 8 fold or greater, 9 fold or greater, 10 fold or greater, 15 fold or greater, 20 fold or greater, 25 fold or greater, 30 fold or greater, 40 fold or greater, 50 fold or greater, 100 fold or greater, etc. In some embodiments, a decrease is assessed relative to an appropriate reference. In some embodiments, inhibition may be complete or “ ”essentially complete, for example to an undetectable level, e.g., to zero or essentially to zero.

Derivative: As used herein, the term “derivative” refers to a structural analogue of a reference substance. That is, a “derivative” is a substance that shows significant structural similarity with the reference substance, for example sharing a core or consensus structure, but also differs in certain discrete ways. In some embodiments, a derivative is a substance that can be generated from the reference substance by chemical manipulation. In some embodiments, a derivative is a substance that can be generated through performance of a synthetic process substantially similar to (e.g., sharing a plurality of steps with) one that generates the reference substance. For example, in some embodiments, a “derivative” of a nucleic acid residue may be or comprise a difference on a nucleotide base, on the sugar or on the phosphate. In some embodiments, a “derivative” of a nucleic acid may be a nucleic acid that contains one or more nucleotides and/or nucleotide analogs not occurring naturally. In some embodiments, a derivative of a nucleic acid is more stable than a comparable nucleic acid lacking the relevant derivatization. In some embodiments, the term “derivative” is used to refer to a nucleic acid sequence that “ ”is a variant with respect to a particular reference sequence; in some such embodiments, such derived (i.e., variant) sequence shows comparable or improved stability and/or translation efficiency relative to its parent reference sequence, for example, when it replaces such parent reference sequence in an RNA molecule.

Detecting: The term “detecting” is used broadly herein to include appropriate means of determining the presence or absence of an entity of interest or any form of measurement of an entity of interest in a sample. Thus, “detecting” may include determining, measuring, assessing, or assaying the presence or absence, level, amount, and/or location of an entity of interest. Quantitative and qualitative determinations, measurements or assessments are included, including semi-quantitative. Such determinations, measurements or assessments may be relative, for example when an entity of interest is being detected relative to a control reference, or absolute. As such, the term “quantifying” when used in the context of quantifying an entity of interest can refer to absolute or to relative quantification. Absolute quantification may be accomplished by correlating a detected level of an entity of interest to known control standards (e.g., through generation of a standard curve). Alternatively, relative quantification can be accomplished by comparison of detected levels or amounts between two or more different entities of interest to provide a relative quantification of each of the two or more different entities of interest, i.e., relative to each other.

Determine: Those of ordinary skill in the art, reading the present specification, will appreciate that a step of “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Encapsulate: The term “encapsulate” or “encapsulation” is used herein to refer to at least a portion of a component is enclosed or surrounded by another material or another component in a composition. In some embodiments, a component can be fully enclosed or surrounded by another material or another component in a composition.

Excipient: As used herein, the term “excipient” refers to a non-therapeutic agent that may be included in a pharmaceutical composition, for example to provide or contribute to a desired property or effect (e.g., desired consistency, delivery, and/or stabilizing effect, etc.). In some embodiments, the term “excipient” is intended to indicate substance(s) which may be present in a pharmaceutical composition and which are not active ingredients such as, e.g., carriers, binders, lubricants, thickeners, surface active agents, preservatives, emulsifiers, buffers, flavoring agents, or colorants. In some embodiments, suitable pharmaceutical excipients to be added to a LNP composition may include, for example, salts, starch, glucose, lactose, sucrose, gelatin, sodium chloride, glycerol, propylene, glycol, water, ethanol and the like.

Encode: As used herein, the term “encode” or “encoding” refers to sequence information of a first molecule that guides production of a second molecule having a defined sequence of nucleotides (e.g., mRNA) or a defined sequence of amino acids. For example, a DNA molecule can encode an RNA molecule (e.g., by a transcription process that includes a DNA-dependent RNA polymerase enzyme). An RNA molecule can encode a polypeptide (e.g., by a translation process). Thus, a gene, a cDNA, or a single-stranded RNA (e.g., an mRNA) encodes a polypeptide if transcription and translation of mRNA corresponding to that gene produces the polypeptide in a cell or other biological system. In some embodiments, a coding region of a single-stranded RNA encoding a target polypeptide agent refers to a coding strand, the nucleotide sequence of which is identical to the mRNA sequence of such a target polypeptide agent. In some embodiments, a coding region of a single-stranded RNA encoding a target polypeptide agent refers to a non-coding strand of such a target polypeptide agent, which may be used as a template for transcription of a gene or cDNA. As is understood in the art, the phrase “nucleic acid encoding a peptide or protein” means that the nucleic acid, if present in the appropriate environment, for example within a cell and/or in a cell-free translation system, can direct the assembly of amino acids to produce the peptide or protein via a process of translation. In some embodiments, a coding is able to interact with cellular translation machinery allowing translation of such coding RNA to yield the encoded peptide or protein.

Endogenous: As used herein “endogenous” refers to material from or produced inside an organism, cell, tissue or system in which it is found.

Exogenous: As used herein, the term “exogenous” refers to a material introduced into or produced outside of an organism, cell, tissue or system in which it is located.

Expression: As is understood in the art, the term “expression” is used to refer to production of a templated nucleic acid (typically an RNA template) and/or of a polypeptide encoded thereby. Thus, in some embodiments, the term may be used to refer to production of RNA, of polypeptide, of RNA and polypeptide; alternatively or additionally, in some embodiments, it may refer to comprises partial expression of nucleic acids. Furthermore, expression may be transient or stable or continuous. As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein. Those skilled in the art will appreciate that the term “expression” or “translation”, when applied to an RNA, typically refers to a process by which a ribosome (e.g., in a cell) reads a strand of coding RNA (e.g. messenger RNA) and directs the assembly of a sequence of amino acids to make an encoded peptide or protein.

Expression control sequence: As will be appreciated by those skilled in the art reading the present disclosure, the term “expression control sequence”, as used herein, refers to a sequence element whose presence and/or identity influences one or more features of expression of another sequence. Often, an expression control sequence is a nucleic acid sequence element, and often it acts in cis. Thus, in some embodiments, an expression control sequence may be, for example, a promoter, an enhancer, a repressing element, a looping site, a termination site, a ribosome-binding sequence, a translation pause signal, and/or another control element(s) which, for example, may control or regulate transcription of a gene and/or or translation of a transcribed RNA. In particular embodiments of the invention, the expression control sequences can be regulated. The precise structure of expression control sequences present in and/or otherwise associated with a particular expressible construct may vary, for example, depending on species or cell type of relevant expression machinery (e.g., RNA polymerase, splicosome, ribosome, etc) but in many embodiments may include 5′-untranscribed and 5′- and 3′-untranslated sequences involved in initiating transcription and translation, respectively. More specifically, in some embodiments, 5′-untranscribed expression control sequences may include a promoter region which encompasses a promoter sequence for transcription control of a functionally linked gene. In some embodiments, expression control sequences may also include enhancer sequences or upstream activator sequences. In many embodiments, an expression control sequence of a DNA molecule may include 5′-untranscribed and 5′- and 3′-untranslated sequences such as TATA box, capping sequence, CAAT sequence and the like.

Fed-batch process: The term “fed-batch process” as used herein refers to a process in which one or more components are introduced into a vessel, e.g., a bioreactor, at some time subsequent to the beginning of a reaction. In some embodiments, one or more components are introduced by a fed-batch process to maintain its concentration low during a reaction. In some embodiments, one or more components are introduced by a fed-batch process to replenish what is depleted during a reaction.

Five prime untranslated region: As used herein, the terms “five prime untranslated region” or “5′ UTR” refer to a sequence of an mRNA molecule that begins at the transcription start site and ends one nucleotide (nt) before the start codon (usually AUG) of the coding region of an RNA.

Fragment: “Fragment”, with reference to a nucleic acid sequence, relates to a part of a nucleic acid sequence, e.g., a sequence which represents less than the parental sequence from which the fragment is derived, e.g., a nucleic acid sequence shortened at the 5′- and/or 3′-end(s), and/or by removal of one or more internal residues. In some embodiments, a fragment of a nucleic acid sequence comprises at least 80%, or in some embodiments at least 90%, 95%, 96%, 97%, 98%, or 99% of the corresponding nucleotide residues from such parental nucleic acid sequence. In many embodiments, a fragment retains one or more properties or attributes of its parental sequence. For example, in some embodiments, a fragment of a translatable RNA is characterized by stability and/or translational efficiency that is at least reasonably comparable to that of its parent. In some embodiments, a nucleic acid whose nucleic acid sequence represents a two or more discontinuous sequences derived from the same parental nucleic acid fused together is considered to be a fragment of that parental nucleic acid.

“Fragment”, with reference to an amino acid sequence (peptide or protein), relates to a part of an amino acid sequence, e.g. a sequence which represents the amino acid sequence shortened at the N-terminus and/or C-terminus and/or missing one or more internal residues. In some embodiments, a fragment shortened at the C-terminus (N-terminal fragment) is obtainable e.g. by translation of a truncated open reading frame that lacks the 3′-end of the open reading frame. In some embodiments, a fragment shortened at the N-terminus (C-terminal fragment) is obtainable e.g. by translation of a truncated open reading frame that lacks the 5′-end of the open reading frame, as long as the truncated open reading frame comprises a start codon that serves to initiate translation. In many embodiments, a fragment of an amino acid sequence comprises e.g. at least 1%, at least 2%, at least 3%, at least 4%, at least 5%, at least 10%, at least 20%, at least 30%, at least 40%, at least 50%, at least 60%, at least 70%, at least 80%, at least 90% of the amino acid residues from an amino acid sequence. In many embodiments, a fragment includes at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80 85, 90 95, 100 or more amino acids, In many embodiments, a fragment retains one or more properties or attributes of its parental sequence. In some embodiments, a polypeptide whose nucleic acid sequence represents a two or more discontinuous sequences derived from the same parental polypeptide fused together is considered to be a fragment of that parental polypeptide.

Functional: As used herein, a “functional” biological molecule is a biological molecule in a form in which it exhibits a property and/or activity by which it is characterized. In some embodiments, a biological molecule may have one function (i.e., monofunctional), two functions (i.e., bifunctional) or many functions (i.e., multifunctional).

Functional analog: In certain embodiments, an “analog” is a “functional analog”. The term “functional analog” refers to an analog of a substance which comprises or shares one or more functions with the reference substance. For example, a functional analog of a nucleoside triphosphate (NTP) shares one or more functions with the reference NTP. For example, a functional analog of GTP shares one or more functions with GTP. For example, a functional analog of CTP shares one or more functions with CTP. For example, a functional analog of ATP shares one or more functions with ATP. For example, a functional analog of UTP shares one or more functions with UTP. In certain embodiments, a functional analog of an NTP is translatable. In certain embodiments, a functional analog of an NTP can be incorporated into a product molecule, e.g., into an RNA instead of, i.e., replacing, the reference NTP. In certain embodiments, the functional analog of an NTP, when incorporated in an RNA molecule, allows translation of the RNA molecule, wherein the functional analog functions as the reference NTP during translation. In some embodiments, a functional analog of an NTP has further characteristics not shared with the reference NTP; for example, it is known that incorporation of Pseudo-UTP and/or N1-Methylpseudo-UTP instead of, i.e., replacing, UTP, may result in RNA with reduced immunogenicity compared to RNA transcribed from the same template using non-modified UTP. In some embodiments, a functional analog of UTP can be incorporated into an RNA molecule instead of UTP and/or is translatable, e.g., as or instead of UTP. Similarly, a functional analog of GTP can be incorporated into an RNA molecule instead of GTP and/or is translatable, e.g., as or instead of GTP. The same applies to CTP and ATP. In certain embodiments, a functional analog of an NTP can be incorporated during synthesis of an RNA molecule at any given position where the respective NTP is expected or predicted, e.g., by the matrix used such as DNA from which the RNA is transcribed.

Functional linkage: As used herein, “functional linkage” or “functionally linked” relates to a connection within a functional relationship. A nucleic acid is “functionally linked” if it is functionally related to another nucleic acid sequence. For example, a promoter is functionally linked to a coding sequence if it influences transcription of said coding sequence. Functionally linked nucleic acids are typically adjacent to one another, where appropriate separated by further nucleic acid sequences, and, in particular embodiments, are transcribed by RNA polymerase to give a single RNA molecule (common transcript). In particular embodiments, a nucleic acid is functionally linked according to the invention to expression control sequences which may be homologous or heterologous with respect to the nucleic acid.

Gene: As used herein, the term “gene” refers to a DNA sequence in a chromosome that codes for a product (e.g., an RNA product and/or a polypeptide product). In some embodiments, a gene includes coding sequence (i.e., sequence that encodes a particular product); in some embodiments, a gene includes non-coding sequence. In some particular embodiments, a gene may include both coding (e.g., exonic) and non-coding (e.g., intronic) sequences. In some embodiments, a gene may include one or more regulatory elements that, for example, may control or impact one or more aspects of gene expression (e.g., cell-type-specific expression, inducible expression, etc.).

Gene product or expression product: As used herein, the term “gene product” or “expression product” generally refers to an RNA transcribed from the gene (pre- and/or post-processing) or a polypeptide (pre- and/or post-modification) encoded by anRNA transcribed from the gene.

Heterologous: The term “heterologous” is used herein to describe an entity relative to a reference and to specify that the entity originated in a source that is different, and/or in association with one or more components that are different, from the relevant reference. As an example, the introduction of one individual's cell into a different individual constitutes a heterologous transplant. A heterologous gene is a gene derived from a source other than the subject.

Homology: As used herein, the term “homology” or “homolog” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or polypeptide molecules are considered to be “homologous” to one another if their sequences are at least 15%, 20%, 25%, 30%, 35%, 40%, 45%, 50%, 55%, 60%, 65%, 70%, 75%, 80%, 85%, 90%, 95%, or 99% identical. In some embodiments, polynucleotide molecules (e.g., DNA molecules and/or RNA molecules) and/or polypeptide molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar”side chains. Substitution of one amino acid for another of the same type may often be considered a “homologous” substitution.

Host cell: As used herein, refers to a cell into which exogenous material (e.g., DNA such as recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, a host cell is a human, monkey, ape, hamster, rat, or mouse cell. In some embodiments, a host cell is eukaryotic. For example, an eukaryotic host cell may be CHO (e.g., CHO K1, DXB-11 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3 A cell, HT1080 cell, myeloma cell, tumor cell, or a cell line derived from an aforementioned cell.

Hybridizing: A nucleic acid is “capable of hybridizing” or “hybridizes” to another nucleic acid if the two sequences are complementary with one another. A nucleic acid is “complementary” to another nucleic acid if the two sequences are capable of forming a stable duplex with one another, e.g., hybridize to one another to form a double-stranded molecule. Complementarity can be total or partial. Those skilled in the art are aware that ability of two sequences to hybridize with one another may depend on conditions (e.g., temperature, pH) and/or presence of other potentially competing sequences. In some embodiments, hybridization is carried out under stringent conditions, so that only highly complementary sequences form stable hybrids. Exemplary such stringent conditions are described, for example, in Molecular Cloning: A Laboratory Manual, J. Sambrook et al., Editors, 2nd Edition, Cold Spring Harbor Laboratory press, Cold Spring Harbor, New York, 1989 or Current Protocols in Molecular Biology, F. M. Ausubel et al., Editors, John Wiley & Sons, Inc., New York. For example, in some embodiments, stringent hybridization may involve incubation of a hybridizing nucleic acid with a membrane containing a complementary nucleic acid at 65° C. in hybridization buffer (3.5×SSC, 0.02% Ficoll, 0.02% polyvinylpyrrolidone, 0.02% bovine serum albumin, 2.5 mM NaH2PO4 (pH 7), 0.5% SDS, 2 mM EDTA). SSC is 0.15 M sodium chloride/0.15 M sodium citrate, pH 7; after such incubation, the membrane to which the DNA has been transferred is washed, for example, in 2×SSC at room temperature and then in 0.1-0.5×SSC/0.1×SDS at temperatures of up to 68° C.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Improved, increased or reduced: As used herein, these terms, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Increase, enhance: As is understood in the art, terms such as “increase” or “enhance” may be used herein to refer to an overall and/or relative increase or enhancement e.g., in an entity, event, frequency, activity, etc. In some embodiments, an increase, for example, may be by about at least 10%, in some embodiments at least 20%, in some embodiments at least 30%, in some embodiments at least 40%, in some embodiments at least 50%, 55%, 65%, 70%, 75%, in some embodiments at least 80%, 85%, 90%, 95% and in some embodiments at least 100% or more. In some embodiments, an increase or enhancement can be 2-fold, 3-fold, 4-fold, 5-fold, 6-fold, 7-fold, 8-fold, 9-fold, 10-fold or more.

In vitro: The term “in vitro” as used herein refers to events that occur in an artificial environment, e.g., in a test tube or reaction vessel (e.g., a bioreactor), in cell culture, etc., rather than within a multi-cellular organism.

In vitro transcription, Transcription: As is known in the art, the terms “transcription” and “transcribing” relate to a process during which a nucleic acid molecule with a particular nucleic acid sequence (the “nucleic acid template”) is read by an RNA polymerase so that the RNA polymerase synthesizes its complementary single-stranded RNA molecule. During transcription, the genetic information in the nucleic acid template is transcribed. In some embodiments, the nucleic acid template is or comprises DNA; however, in some embodiments, e.g. in the case of transcription from an alphaviral nucleic acid template, the nucleic acid template may be or comprise RNA. In some embodiments, a nucleic acid template may include one or more residues that is neither DNA nor RNA and/or that is a DNA or RNA analog (e.g., that contains one or more modifications—e.g., a backbone modification or a base modification) relative to canonical DNA or RNA. In many embodiments, a transcribed RNA may be translated into protein. In many embodiments, as will be clear from context, the term “transcription” used herein refers to “in vitro transcription”. As used herein, the term “in vitro transcription” or “IVT” refers to the process whereby transcription occurs in vitro (i.e., outside of an organism and typically in a non-cellular system) to produce a synthetic RNA product; in many embodiments, the present disclosure describes IVT to generate and RNA product for use in certain applications, including, e.g., production of protein or polypeptides. In some embodiments, an produced RNA product can be translated in vitro, or can be introduced directly into cells, where in some embodiments it can be can be translated. In certain particular embodiments, a produced RNA product is of sufficient scale and/or quality for administration to an organism, and in some embodiments, a human (e.g., as a pharmaceutically active RNA). In some embodiments, an RNA product may be selected from, e.g., but not limited to, mRNAs, antisense RNA molecules, shRNA molecules, long non-coding RNA molecules, ribozymes, aptamers, guide RNAs (e.g., for CRISPR), ribosomal RNAs, small nuclear RNAs, small nucleolar RNAs, and the like. An IVT reaction typically utilizes a DNA template (e.g., a linear DNA template) as described and/or utilized herein, ribonucleotides (e.g., non-modified ribonucleotide triphosphates or modified ribonucleotide triphosphates), and an appropriate RNA polymerase. In some embodiments, cloning vectors are applied for the generation of transcripts. In some such embodiments, cloning vectors are designated as “transcription vectors” (which are according to the present invention encompassed by the term “vector”). In some embodiments, cloning vectors may be plasmids. In some embodiments, an RNA is in vitro transcribed RNA (IVT-RNA) and may be obtained by in vitro transcription of an appropriate DNA template. Those skilled in the art are aware of a variety of promoter sequences that can appropriately be used for controlling transcription by a relevant RNA polymerase. In some embodiments, a DNA template for in vitro transcription may be obtained by cloning of a nucleic acid, such as for example a cDNA, and introducing it into an appropriate vector for in vitro transcription. In some embodiments, a cDNA may be obtained by reverse transcription of RNA.

In vitro transcription RNA composition: As used herein, the term “in vitro transcription RNA composition” refers to a composition comprising RNA synthesized by in vitro transcription. In some embodiments, such a composition can comprise excess in vitro transcription reagents (including, e.g., ribonucleotides and/or capping agents), nucleic acids or fragments thereof such as DNA templates or fragments thereof, polypeptides or fragments thereof such as recombinant enzymes or host cell proteins or fragments thereof, and/or other impurities. In some embodiments, an in vitro transcription RNA composition may have been treated and/or processed prior to a purification processes that ultimately produces an RNA transcript preparation comprising RNA transcript at a desired concentration in an appropriate buffer for formulation and/or further manufacturing and/or processing. For example, in some embodiments, an in vitro transcription RNA composition may have been treated to remove or digest DNA template (e.g., using a DNase). In some embodiments, an in vitro transcription RNA composition may have been treated to remove or digest polypeptides (e.g., enzymes such as RNA polymerases, RNase inhibitors, etc.) present in an in vitro transcription reaction (e.g., using a protease). Thus, in some embodiments, following RNA transcription, a DNA template can be removed or separated from a composition comprising RNA; those skilled in the art are aware of a variety of methods, e.g., DNA hydrolysis, by which such removal may be accomplished. In some embodiments, an RNase inhibitor may be added during DNA removal or digestion to protect RNA from potential degradation. In some embodiments, an in vitro transcription RNA composition may have been treated to remove or digest peptides (e.g., enzymes such as RNA polymerases, RNase inhibitors, etc.) present in an in vitro transcription reaction (e.g., using a protease).

In vivo: As used herein, the term “in vivo” refers to events that occur within a multi-cellular organism, such as a human and a non-human animal.

Isolated: The term “isolated” as used herein, typically refers to a molecule or other entity which is substantially free of other components, such as other cellular material; in some embodiments, an “isolated” entity is substantially free of components with which it was previously associated (e.g., when initially generated). In certain embodiments, the term “isolated nucleic acid” as used herein refers to a nucleic acid that has been (i) amplified in vitro, for example by polymerase chain reaction (PCR), (ii) recombinantly produced by cloning, (iii) purified, for example by cleavage and gel-electrophoretic fractionation, or (iv) synthesized, for example by chemical synthesis or IVT. In some embodiments, an isolated nucleic acid is a nucleic acid available to manipulation by recombinant techniques.

Linked, fused, fusion: As used herein, the terms “linked,” “fused”, or “fusion” are used interchangeably. These terms refer to the joining together (e.g., by covalent linkage) of two or more elements or components or domains, e.g., domains from two different proteins or nucleic acid molecules.

Messenger-RNA, mRNA: According to the invention, the term “mRNA” means “messenger-RNA” and relates to a transcript which is typically generated from a template (e.g., a DNA template) and encodes a peptide or protein. Typically, an mRNA comprises a 5′ UTR, a protein coding region, a 3′ UTR, and a poly(A) sequence. In some embodiments, mRNA may be generated by in vitro transcription from a DNA template as described herein. In some embodiments, mRNA may be modified, for example by stabilizing modifications and/or capping. In some embodiments, a nucleic acid such as RNA, e.g. mRNA, may encode a peptide or protein. Accordingly, in some embodiments, a transcribable nucleic acid sequence or a transcript thereof may contain an open reading frame (ORF) encoding a peptide or protein.

Nanoparticle: As used herein, the term “nanoparticle” refers to a particle having a diameter of less than 1000 nanometers (nm). In some embodiments, a nanoparticle has a diameter of less than 300 nm, as defined by the National Science Foundation. In some embodiments, a nanoparticle has a diameter of less than 100 nm as defined by the National Institutes of Health. In some embodiments, a nanoparticle has a diameter of less than 80 nm as defined by the National Institutes of Health. In some embodiments, a nanoparticle comprises one or more enclosed compartments, separated from the bulk solution by a membrane, which surrounds and encloses a space or compartment.

Nucleic acid/Polynucleotide: The term “nucleic acid” as used herein, refers to a polymer comprising two or more nucleotide or nucleotide analog residues. In some embodiments, a nucleic acid may include one or more resides or linkages that is modified relative to a naturally-occurring DNA or RNA residue. For example, in some embodiments, a nucleic acid may have one or more modifications of a base, sugar or backbone (e.g., phosphate) relative to a naturally-occurring DNA or RNA reside. In some embodiments, a nucleic acid molecule refers to a nucleic acid which is or comprises deoxyribonucleic acid (DNA) or ribonucleic acid (RNA). In some embodiments, nucleic acids may be or comprise, or may have sequences found in, genomic DNA, cDNA, mRNA, viral RNA, siRNA, miRNA, shRNA, recombinantly prepared and chemically synthesized molecules. In some embodiment, the term “nucleic acid” refers to a polymer of at least 2 residues or more, including, e.g., at least 3 residues, at least 4 residues, at least 5 residues, at least 6 residues, at least 7 residues, at least 8 residues, at least 9 residues, at least 10 residues, or more. In some embodiments, a nucleic acid is or comprises DNA. In some embodiments, a nucleic acid is or comprises RNA. In some embodiments, a nucleic acid is or comprises peptide nucleic acid (PNA). In some embodiments, a nucleic acid is or comprises a single stranded nucleic acid. In some embodiments, a nucleic acid is or comprises a double-stranded nucleic acid. In some embodiments, a nucleic acid comprises both single and double-stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, I-methyl-pseudouridine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Nucleic acid sequence: According to the invention “nucleic acid sequence” refers to the sequence of residues in a nucleic acid, e.g. a ribonucleic acid (RNA) or a deoxyribonucleic acid (DNA). In some embodiments, the term is used in reference to the sequence of an entire nucleic acid molecule (such as to the single strand of an entire nucleic acid molecule); in some embodiment, the term is used to refer to a sequence that represents a part (e.g. a fragment) thereof.

Nucleotide: The term “nucleotide” is used herein as commonly understood in the art and can refer to nucleoside monophosphate, nucleoside diphosphate and nucleoside triphosphate.

Pharmaceutical grade: The term “pharmaceutical grade” as used herein refers to standards for chemical and biological drug substances, drug products, dosage forms, compounded preparations, excipients, medical devices, and dietary supplements, established by a recognized national or regional pharmacopeia (e.g., The United States Pharmacopeia and The Formulary (USP-NF)).

Polypeptide: The term “polypeptide”, as used herein, typically has its art-recognized meaning of a polymer of at least three amino acids or more. Those of ordinary skill in the art will appreciate that the term “polypeptide” is intended to be sufficiently general as to encompass not only polypeptides having a complete sequence recited herein, but also to encompass polypeptides that represent functional, biologically active, or characteristic fragments, portions or domains (e.g., fragments, portions, or domains retaining at least one activity) of such complete polypeptides. In some embodiments, polypeptides may contain L-amino acids, D-amino acids, or both and/or may contain any of a variety of amino acid modifications or analogs known in the art. Useful modifications include, e.g., terminal acetylation, amidation, methylation, etc. In some embodiments, polypeptides may comprise natural amino acids, non-natural amino acids, synthetic amino acids, and combinations thereof (e.g., may be or comprise peptidomimetics). In some embodiments, a polypeptide may be or comprise an enzyme. In some embodiments, a polypeptide may be or comprise a polypeptide antigen. In some embodiments, a polypeptide may be or comprise an antibody agent. In some embodiments a polypeptide may be or comprise a cytokine.

Primary structure: The term “primary structure”, as used herein with reference to a nucleic acid molecule, refers to the linear sequence of monomer residues.

Promoter, promoter region: The term “promoter” or “promoter region” refers to a nucleic acid sequence which directs synthesis of a transcript, e.g. a transcript comprising a coding sequence, for example by providing a recognition and binding site for RNA polymerase. In some embodiments, a promoter region may include further recognition or binding sites for further factors involved in regulating transcription of said gene. In some embodiments promoter may control transcription of a prokaryotic or eukaryotic gene. In some embodiments, a promoter may be “inducible” and initiate transcription in response to an inducer; in some embodiments, a promoter may be “constitutive” if transcription is not controlled by an inducer or cell-type-specific promoter. In some embodiments, an inducible promoter is expressed only to a very small extent or not at all, if an inducer is absent; when the inducer is present, promoter is “switched on” or the level of transcription is increased, typically mediated by binding of a specific transcription factor.

Pure or Purified: As used herein, an agent or entity is “pure” or “purified” if it is substantially free of other components. For example, a preparation that contains more than about 90% of a particular agent or entity is typically considered to be a pure preparation. In some embodiments, an agent or entity is at least 91%, at least 92%, at least 93%, at least 94%, at least 95%, at least 96%, at least 97%, at least 98%, or at least 99% pure in a preparation.

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides (analogs) may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. In some embodiments, a modified ribonucleotide maintains at least one function of the corresponding nonmodified ribonucleotide. The term “ribonucleotide” can encompass ribonucleotide triphosphates including modified and non-modified ribonucleotide triphosphates.

Ribonucleic acid (RNA): As used herein, the term “RNA” refers to a polymer of ribonucleotides; the term “RNA” or “RNA molecule” relates to a molecule which comprises ribonucleotide residues. In some embodiments, an “RNA” is entirely or substantially composed of ribonucleotide residues. As those skilled in the art are aware, a canonical “ribonucleotide” is a nucleotide with a hydroxyl group at the 2′-position of a β-D-ribofuranosyl group. The term “RNA” can comprise double-stranded RNA, single stranded RNA, isolated RNA such as partially or completely purified RNA, essentially pure RNA, synthetic RNA, and recombinantly generated RNA such as modified RNA which differs from naturally occurring RNA by addition, deletion, substitution and/or alteration of one or more nucleotides. In some embodiments, an RNA may be modified relative to a reference (e.g., a naturally occurring RNA), for example, by addition of non-nucleotide material, such as to the end(s) of an RNA or internally, for example at one or more nucleotides of the RNA. In some embodiments, one or more residues or linkages in an RNA molecule may be or comprise a non-standard residue or linkage, such as a non-naturally occurring nucleotide or a chemically synthesized nucleotide or a deoxynucleotide; in some embodiments, such an RNA may be referred to as an analog, e.g., an analog of a naturally-occurring RNA. In some embodiments, an RNA is single stranded. In some embodiments, an RNA is double stranded. In some embodiments, an RNA comprises both single and double stranded portions. In some embodiments, an RNA can comprise a backbone structure as described in the definition of “Nucleic acid/Polynucleotide” above. An RNA can be a regulatory RNA (e.g., siRNA, microRNA, etc.), or a messenger RNA (mRNA). In some embodiments where an RNA is a mRNA. In some embodiments where an RNA is a mRNA, a RNA typically comprises at its 3′ end a poly(A) region. In some embodiments where an RNA is a mRNA, an RNA typically comprises at its 5′ end an art-recognized cap structure, e.g., for recognizing and attachment of a mRNA to a ribosome to initiate translation. In some embodiments, an RNA is a synthetic RNA. Synthetic RNAs include RNAs that are synthesized in vitro (e.g., by enzymatic synthesis methods and/or by chemical synthesis methods). In some embodiments, an RNA is a single-stranded RNA. In some embodiments, a single-stranded RNA may comprise self-complementary elements and/or may establish a secondary and/or tertiary structure. Those skilled in the art will appreciate that the term “single-stranded RNA” generally refers to an RNA molecule to which no complementary nucleic acid molecule (typically no complementary RNA molecule) is associated. In some embodiments, a single-stranded RNA may contain self-complementary sequences that allow parts of the RNA to fold back and to form secondary structure motifs including without limitation base pairs, stems, stem loops and/or bulges as is known in the art. Those skilled in the art will understand from context when a single-stranded RNA is referred to as “encoding,” whether the reference is to a coding strand sequence or its complement. In some embodiments, a single-stranded RNA can be a self-amplifying RNA (also referred to as self-replicating RNA).

Recombinant: as used herein, the term “recombinant”, when used to refer to a polypeptide, is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.). In some embodiments, nucleic acids described herein may be recombinant and/or isolated molecules.

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

RNA polymerase: As used herein, the term “RNA polymerase” refers to an enzyme that catalyzes polyribonucleotide synthesis by addition of ribonucleotide units to a nucleotide chain using DNA or RNA as a template. As will be clear from context, the term refers to either a complete enzyme as it occurs in nature, or an isolated, active catalytic or functional domain, or fragment thereof. In some embodiments, an RNA polymerase enzyme initiates synthesis at the 3′-end of a primer or a nucleic acid strand, or at a promoter sequence, and proceeds in the 5′-direction along the target nucleic acid to synthesize a strand complementary to the target nucleic acid until synthesis terminates.

RNA transcript preparation: The term “RNA transcript preparation” as used herein refers to a preparation comprising RNA transcript that is purified from an in vitro transcription RNA composition described herein. In some embodiments, an RNA transcript preparation is a preparation comprising pharmaceutical-grade RNA transcript. In some embodiments, an RNA transcript preparation is a preparation comprising RNA transcript, which its one or more product quality attributes are characterized and determined to meet a release and/or acceptance criteria (e.g., as described herein). Examples of such product quality attributes include, but are not limited to appearance, RNA length, identity of drug substance as RNA, RNA integrity, RNA sequence, RNA concentration, pH, osmolality, residual DNA template, residual double stranded RNA, bacterial endotoxins, bioburden, and combinations thereof.

Room temperature: As used herein, the term “room temperature” refers to an ambient temperature. In some embodiments, a room temperature is about 15° C., 16° C., 17° C., 18° C., 19° C., 20° C., 21° C., 22° C., 23° C., 24° C., 25° C., 26° C., 27° C., 28° C., 29° C., 30° C., preferably about 18° C.-30° C., e.g., about 18° C.-25° C., or about 20° C.-25° C., or about 20-30° C., or about 23-27° C. or about 25° C.

Sample: As used herein, the term “sample” typically refers to an aliquot of material obtained or derived from a source of interest, e.g., as described herein. In some embodiments, a source of interest is a biological or environmental source. In some embodiments, a source of interest may be or comprise a cell or an organism, such as a microbe, a plant, or an animal (e.g., a mouse). In some embodiments, a source of interest is or comprises biological tissue or fluid. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In some embodiments, a sample is or comprises cells obtained from a subject. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Secondary structure: As is understood in the art, the term “secondary structure” is used to refer to interactions between bases in nucleic acid molecules. Thus, secondary structure can be described as a two-dimensional representation of a nucleic acid molecule that reflects base pairings. The term is often used in reference to intra-molecular base pairing interactions in a single-stranded molecule, e.g., a single-stranded RNA. Indeed, many single stranded nucleic acid molecules, and particularly single stranded RNA molecules are characterized by regions of (intramolecular) base pairs. According to the invention, the term “secondary structure” comprises structural motifs including without limitation base pairs, stems, stem loops, bulges, loops such as interior loops and multi-branch loops. The secondary structure of a nucleic acid molecule can be represented by a two-dimensional drawing (planar graph), showing base pairings (for further details on secondary structure of RNA molecules, see Auber et al., (2006), J. Graph Algorithms Appl., 10: 329-351). As described herein, the secondary structure of certain RNA molecules is relevant in the context of the present invention. Secondary structure of a nucleic acid molecule, particularly of a single-stranded RNA molecule, may be determined by prediction using the web server for RNA secondary structure prediction (http://rna.urmc.rochester.edu/RNAstructureWeb/Servers/Predict1/Predict1.html).

Stable: The term “stable,” when applied to nucleic acids and/or compositions comprising nucleic acids, e.g., encapsulated in lipid nanoparticles, means that such nucleic acids and/or compositions maintain one or more aspects of their characteristics (e.g., physical and/or structural characteristics, function, and/or activity) over a period of time under a designated set of conditions (e.g., pH, temperature, light, relative humidity, etc.). In some embodiments, such stability is maintained over a period of time of at least about one hour; in some embodiments, such stability is maintained over a period of time of about 5 hours, about 10 hours, about one (1) day, about one (1) week, about two (2) weeks, about one (1) month, about two (2) months, about three (3) months, about four (4) months, about five (5) months, about six (6) months, about eight (8) months, about ten (10) months, about twelve (12) months, about twenty-four (24) months, about thirty-six (36) months, or longer. In some embodiments, such stability is maintained over a period of time within the range of about one (1) day to about twenty-four (24) months, about two (2) weeks to about twelve (12) months, about two (2) months to about five (5) months, etc. In some embodiments, such stability is maintained under an ambient condition (e.g., at room temperature and ambient pressure). In some embodiments, such stability is maintained under a physiological condition (e.g., in vivo or at about 37° C. for example in serum or in phosphate buffered saline). In some embodiments, such stability is maintained under cold storage (e.g., at or below about 4° C., including, e.g., −20° C., or −70° C.). In some embodiments, such stability is maintained when nucleic acids and/or compositions comprising the same are protected from light (e.g., maintaining in the dark).

As an example, in some embodiments, the term “stable” is used in reference to a nanoparticle composition (e.g., a lipid nanoparticle composition). In such embodiments, a stable nanoparticle composition (e.g., a stable nanoparticle composition) and/or component(s) thereof maintain one or more aspects of its characteristics (e.g., physical and/or structural characteristics, function(s), and/or activity) over a period of time under a designated set of conditions. For example, in some embodiments, a stable nanoparticle composition (e.g., a lipid nanoparticle composition) is characterized in that average particle size, particle size distribution, and/or polydispersity of nanoparticles is substantially maintained (e.g., within 10% or less, as compared to the initial characteristic(s)) over a period of time (e.g., as described herein) under a designated set of conditions (e.g., as described herein). In some embodiments, a stable nanoparticle composition (e.g., a lipid nanoparticle composition) is characterized in that no detectable amount of degradation products (e.g., associated with hydrolysis and/or enzymatic digestion) is present after it is maintained under a designated set of conditions (e.g., as described herein) over a period of time.

Stability of RNA: The term “stability of RNA” is commonly used herein in reference to the “half-life” of RNA. “Half-life” relates to the period of time which is needed to eliminate half of the activity, amount, or number of molecules. In many embodiments, the half-life of an RNA is indicative of its stability. Those skilled in the art will appreciate that half-life of RNA may often influence the “duration of expression” of the RNA; typically, an RNA having a long half-life will be expressed for an extended time period relative to an RNA with a shorter half-life.

Stem loop, hairpin: As used herein, the terms “stem loop” or “hairpin” or “hairpin loop” refer to a particular secondary structure of a nucleic acid molecule, typically a single-stranded nucleic acid molecule, such as a single-stranded RNA. The particular secondary structure represented by the stem loop consists of a consecutive nucleic acid sequence comprising a stem and a loop (e.g., a terminal loop), also called hairpin loop, wherein the stem is formed by two neighbored entirely or partially complementary sequence elements; which are separated by a short sequence (e.g. 3-10 nucleotides), which forms the loop of the stem-loop structure. The two neighbored entirely or partially complementary sequences may be defined as e.g. stem loop elements stem 1 and stem 2. The stem loop is formed when these two neighbored entirely or partially reverse complementary sequences, e.g. stem loop elements stem 1 and stem 2, form base-pairs with each other, leading to a double-stranded nucleic acid sequence comprising an unpaired loop at its terminal ending formed by the short sequence located between stem loop elements stem 1 and stem 2. Thus, a stem loop comprises two stems (stem 1 and stem 2), which—at the level of secondary structure of the nucleic acid molecule—form base pairs with each other, and which—at the level of the primary structure of the nucleic acid molecule—are separated by a short sequence that is not part of stem 1 or stem 2. For illustration, a two-dimensional representation of the stem loop resembles a lollipop-shaped structure. The formation of a stem-loop structure involves a sequence that can fold back on itself to form a paired double strand; the paired double strand is formed by stem 1 and stem 2. The stability of paired stem loop elements is typically determined by the length, the number of nucleotides of stem 1 that are capable of forming base pairs (preferably canonical base pairs, more preferably Watson-Crick base pairs) with nucleotides of stem 2, versus the number of nucleotides of stem 1 that are not capable of forming such base pairs with nucleotides of stem 2 (mismatches or bulges). If a given nucleic acid sequence is characterized by a stem loop, the respective complementary nucleic acid sequence is typically also characterized by a stem loop. A stem loop is typically formed by single-stranded RNA molecules.

Synthetic: As used herein, the term “synthetic” refers to an entity that is artificial, or that is made with human intervention, or that results from synthesis rather than naturally occurring. For example, in some embodiments, a synthetic nucleic acid or polynucleotide refers to a nucleic acid molecule that is chemically synthesized, e.g., in some embodiments by solid-phase synthesis. In some embodiments, the term “synthetic” refers to an entity that is made outside of biological cells. For example, in some embodiments, a synthetic nucleic acid or polynucleotide refers to a nucleic acid molecule (e.g., an RNA) that is produced by in vitro transcription using a template.

Template: As used herein, the terms “template” or “nucleic acid template” or “template nucleic acid” generally refer to a nucleic acid sequence that may be replicated or transcribed. In some embodiments, a template is DNA. In some embodiments, a DNA template is a linear DNA molecule. In some embodiments, a DNA template is a circular DNA molecule. DNA can be obtained or generated using methods known in the art, including, e.g., gene synthesis, recombinant DNA technology, or a combination thereof. In some embodiments, a DNA template comprises a nucleotide sequence coding for a transcribed region of interest (e.g., coding for a RNA described herein) and a promoter sequence that is recognized by an RNA polymerase selected for use in in vitro transcription such as the RNA polymerases described herein. In some embodiments, the DNA template encodes one or more elements of a product such as an RNA, e.g., 5′ UTR, 3′ UTR, open reading frame (e.g., coding for a peptide or protein of interest such as an antigen), poly(A)-tail, etc. In some embodiments, a DNA template encodes all elements of a product RNA. In some embodiments, a DNA template does not encode all elements of the product RNA, e.g., the DNA template may not encode a poly(A)-tail and such poly(A)-tail may be added enzymatically to the RNA after transcription as described herein.

Tertiary structure: As used herein, the term “tertiary structure”, with reference to a nucleic acid molecule, refers to the three-dimensional structure of a nucleic acid molecule, as defined by the atomic coordinates.

Three prime untranslated region: As used herein, the terms “three prime untranslated region” or “3′ UTR” refer to the sequence of an mRNA molecule that begins following the stop codon of the coding region of an open reading frame sequence. In some embodiments, the 3′ UTR begins immediately after the stop codon of the coding region of an open reading frame sequence. In other embodiments, the 3′ UTR does not begin immediately after stop codon of the coding region of an open reading frame sequence

Threshold level (e.g., acceptance criteria): As used herein, the term “threshold level” refers to a level that is used as a reference to attain information on and/or classify the results of a measurement, for example, the results of a measurement attained in an assay. For example, in some embodiments, a threshold level means a value measured in an assay that defines the dividing line between two subsets of a population (e.g. a batch that satisfy quality control criteria vs. a batch that does not satisfy quality control criteria). Thus, a value that is equal to or higher than the threshold level defines one subset of the population, and a value that is lower than the threshold level defines the other subset of the population. A threshold level can be determined based on one or more control samples or across a population of control samples. A threshold level can be determined prior to, concurrently with, or after the measurement of interest is taken. In some embodiments, a threshold level can be a range of values.

Transcriptional efficiency: The term “transcription efficiency” relates to the amount of transcription product produced from a template molecule within a particular period of time.

Translation efficiency: The term “translation efficiency” relates to the amount of translation product provided by an RNA molecule within a particular period of time.

Variant: The term “variant” when used with respect to, for example, nucleic acid and amino acid sequences, includes any variants, in particular mutants, viral strain variants, splice variants, conformations, isoforms, allelic variants, species variants and species homologs, in particular those which are naturally present. An allelic variant relates to an alteration in the normal sequence of a gene, the significance of which is often unclear. Complete gene sequencing often identifies numerous allelic variants for a given gene. With respect to nucleic acid molecules, the term “variant” includes degenerate nucleic acid sequences, wherein a degenerate nucleic acid according to the invention is a nucleic acid that differs from a reference nucleic acid in codon sequence due to the degeneracy of the genetic code. A species homolog is a nucleic acid or amino acid sequence with a different species of origin from that of a given nucleic acid or amino acid sequence. A virus homolog is a nucleic acid or amino acid sequence with a different virus of origin from that of a given nucleic acid or amino acid sequence. In some embodiments, nucleic acid variants may include single or multiple nucleotide deletions, additions, mutations, substitutions and/or insertions in comparison with the reference nucleic acid. Deletions include removal of one or more nucleotides from the reference nucleic acid. Addition variants comprise 5- and/or 3′-terminal fusions of one or more nucleotides, such as 1, 2, 3, 5, 10, 20, 30, 50, or more nucleotides. In the case of substitutions, at least one nucleotide in the sequence is removed and at least one other nucleotide is inserted in its place (such as transversions and transitions). Mutations may include abasic sites, crosslinked sites, and chemically altered or modified bases. Insertions include the addition of at least one nucleotide into the reference nucleic acid.

Vector: As used herein, the term “vector” refers to a nucleic acid molecule capable of transporting another nucleic acid to which it has been linked. Vectors comprise plasmids, cosmid vectors, phagemids such as lambda phage, virus genomes including retroviral, adenoviral or baculoviral vectors, artificial chromosome vectors such as bacterial artificial chromosomes (BAC), yeast artificial chromosomes (YAC), or P1 artificial chromosomes (PAC) and functional portions thereof. One type of vector is a “plasmid”, which refers to a circular double stranded DNA into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Expression vectors comprise plasmids as well as viral vectors and generally contain a desired coding sequence and appropriate non-coding sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

### DETAILED DESCRIPTION OF CERTAIN EMBODIMENTS

Although certain embodiments of the present invention are described in detail below, it is to be understood that this invention is not limited to the particular methodologies, protocols and reagents described herein as these may vary. It is also to be understood that terminology used herein is for the purpose of describing particular embodiments only, and is not intended to limit the scope of the present invention which will be defined by the appended claims. Unless defined otherwise, all technical and scientific terms used herein have the same meanings as commonly understood by one of ordinary skill in the art.

In some embodiments, practice of the present invention will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989). Indeed, in many embodiments, standard techniques may be used, for example, for recombinant DNA production and/or manipulation, oligonucleotide synthesis, tissue culture and transformation (e.g., electroporation, lipofection), etc. Those skilled in the art, reading the present disclosure, will appreciate where enzymatic reactions and/or purification techniques may be performed according to manufacturer's specifications or as commonly accomplished in the art or as described herein. The foregoing techniques and procedures may be generally performed according to conventional methods well known in the art and/or as described in various general and more specific references that are cited and discussed throughout the present specification. See e.g., Green and Sambrook, Molecular Cloning: A Laboratory Manual (4th ed., Cold Spring Harbor Laboratory Press, Cold Spring Harbor, N.Y. (2012)), which is incorporated herein by reference for any purpose.

In the following description, certain elements of the present invention will be described. These elements may be discussed with specific embodiments, however, it should be understood that they may be combined in any manner and in any number to create additional embodiments. The variously described examples and particular embodiments should not be construed to limit the present invention to only the explicitly described embodiments. This description should be understood to disclose and encompass embodiments which combine the explicitly described embodiments with any number of the disclosed and/or preferred elements. Furthermore, any permutations and combinations of all described elements in this application should be considered disclosed by this description unless the context indicates otherwise.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present invention was not entitled to antedate such disclosure.

### Batch, Fed-Batch

The present disclosure provides certain technologies for producing RNA molecules, and particularly for producing them via in vitro transcription (IVT) using a “batch” reaction. Those skilled in the art will appreciate that the term “batch” or “batch reaction”, or similar term, is used to refer to a reaction, such as a transcription reaction, wherein at least one discrete supplementation event takes place for at least one component, e.g., for at least one NTP such as UTP and/or GTP, or a functional analog thereof, and optionally for one or more other component(s) (e.g., components of the reaction mix discussed herein), optionally multiple components supplemented in the same discrete supplementation event. In some embodiments, supplementing the reaction mix comprises supplementing UTP or a functional analog thereof. In some embodiments, supplementing the reaction mix comprises supplementing UTP or a functional analog thereof and GTP or a functional analog thereof. In some embodiments, supplementing the reaction mix comprises supplementing further components such as ATP or a functional analog thereof and/or CTP or a functional analog thereof and/or one or more salts and/or one or more enzymes such as a polymerase and/or one or more 5′ cap nucleotides and/or one or more other components of a reaction mix described herein such as transcription buffer, RNase inhibitor, DNA template, 5′ cap and/or 5′ cap analog, etc. In some embodiments, the reaction mix is supplemented more than once during the course of the transcription and/or capping reaction.

In certain embodiments, a fed-batch process is used in a method of producing an RNA or a composition comprising RNA. The term “fed-batch process” or “fed-batch reaction” or similar terms refer to a process or reaction, wherein a starting reaction mix in which part or all of the components are present (batch reaction) and wherein the reaction is occasionally supplemented with one or more components during the course of the reaction. In some embodiments, one or more components such as UTP or a functional analog thereof and/or GTP or a functional analog thereof are supplemented, e.g., introduced by a fed-batch process, to maintain their concentrations low during a reaction or to restore the ration of their initial concentration to the initial concentration of CTP and/or ATP, or a functional analog thereof. In some embodiments, one or more components such as UTP or a functional analog thereof and/or GTP or a functional analog thereof are supplemented, e.g., introduced by a fed-batch process to replenish what is depleted during a reaction. “Supplementing the reaction mix” refers to the supplementation of a component in discrete amounts to the reaction after the reaction has commenced. However, supplementing a fed-batch reaction is not limited to supplementation with discrete amounts. In some embodiments, supplementing comprises supplementing by continuous flow, i.e., continuous supplementation of one or more components of the reaction mix during the course of the transcription and/or capping reaction.

In some embodiments a fed-batch process involves using a starting reaction mix, wherein all nucleotide triphosphates which are part of the RNA to be synthesized are present; in other embodiments, a fed-batch process involves using a starting reaction mix wherein not all nucleotide triphosphates which are part of the RNA to be synthesized are present.

In some embodiments, a starting reaction mix contains ATP, GTP, CTP and UTP, or functional analogs thereof. In some embodiments, a starting reaction mix used is substantially free of ATP or functional analogs thereof. In some embodiments, a starting reaction mix is substantially free of GTP or functional analogs thereof. In some embodiments, a starting reaction mix is substantially free of CTP or functional analogs thereof. In some embodiments, a starting reaction mix is substantially free of UTP or functional analogs thereof. It is understood that, when the RNA to be synthesized is predicted (e.g., according to the template used) to comprise a component which is not present in the starting reaction mix, this component has to be supplemented in order to synthesize said RNA.

In a capping and transcription reaction, for example, the reaction commences when an RNA polymerase mediates the formation of a covalent bond between a nucleotide and a cap analog. It will be understood that one difference between a capping and transcription reaction and a transcription reaction without capping is the presence of a component that provides the cap structure to the 5′ end of a transcript such as a 5′ cap or 5′ cap analog described herein.

The present disclosure provides technologies involving a fed-batch processes, wherein at least one component of a reaction mix is present in a limiting amount. “Limiting amount” means that the limiting reaction component is present in an amount, e.g., in a starting concentration, that limits one or more of the time until the reaction stops, the amount of product generated by the process until the reaction stops, and the rate of transcription. For example, in some embodiments, the amount of the limiting reaction component, e.g., UTP, or a functional analog thereof, and/or GTP, or a functional analog thereof, limits one or more of the time until a transcription and/or capping reaction stops, the amount of product, e.g., RNA, produced until the transcription and/or capping reaction stops, and/or the rate of the transcription and/or capping reaction, e.g., the rate at which the reactants are converted into product.

In some embodiments, one or more reaction components are added continuously to the reaction, and one or more components such as one or more limiting components are added periodically to the reaction such as by a fed-batch process. In certain embodiments, a component such as a limiting component, e.g., a limiting nucleotide, is supplemented to the reaction by a fed-batch process periodically or intermittently. The term “periodically” means “occurring at intervals” that can be “regular” meaning “fixed” with respect to characteristics, such as time and/or concentration level in the reaction. The term “intermittently” means “occurring at intervals”. “Intervals” refers both to regular and irregular intervals. It will be understood that “intermittent” supplementation of a component can also be “periodic”. It will further be understood that intermittent introduction or supplementation of a component to a reaction means at least one time, while “periodic” introduction or supplementation of a component is at least two times (to define the “regular interval”). In some embodiments, a component such as a limiting component, e.g., UTP and/or GTP, or a functional analog thereof, may be supplemented, supplemented at least, or supplemented at most 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 or more times, or any range derivable therein, during the course of a transcription and/or capping reaction. In some embodiments, UTP or a functional analog thereof is supplemented at least once. In some embodiments, UTP or a functional analog thereof is supplemented at least twice. In further embodiments, UTP or a functional analog thereof is introduced intermittently or periodically into the reaction between three times and 50 times. In some embodiments, such periodic supplementation may be performed as one or more bolus or batch addition(s), including, e.g., 1, 2, 3, 4, 5, 6, 7, 8, 9, 10, 11, 12, 13, 14, 15 or more bolus or batch addition(s). In some embodiments, such periodic supplementation may be performed by a fed-batch process. Supplementing may also comprise supplementing a composition comprising UTP or a functional analog thereof and comprising further components such as buffer, polymerase, CTP or a functional analog thereof, GTP or a functional analog thereof, ATP or a functional analog thereof, or other components that may be present in a transcription reaction mix as described herein. In some embodiments, a composition for supplementing is essentially free of CTP and/or ATP or functional analogs thereof.

### DS RNA Contaminants

As known in the art, during synthesis of mRNA by a transcription reaction, e.g., in vitro transcription (IVT), e.g., using T7 RNA polymerase or another RNAP, significant amounts of aberrant products, including double-stranded RNA (dsRNA) (e.g., involving hybridization of distinct RNA molecules) are produced due to unconventional activity of the enzyme. dsRNA has been shown to induce inflammatory cytokines and activates effector enzymes leading to protein synthesis inhibition.

As noted herein, dsRNA contamination of in vitro transcription reactions can be problematic. At least two different types of dsRNA contaminants have been described: (i) short dsRNAs in which antisense fragments base pair with the RNA transcript of interest (e.g., an mRNA product) and (ii) nearly full-length dsRNAs. Both can be generated by promotor dependent or promotor-independent RNAP (e.g., T7) activity; some may be influenced by termination sites within the template.

Without wishing to be bound by theory, we note that various mechanisms have been proposed for production of dsRNA during in vitro transcription. For example, in some instances, short RNA transcripts (e.g., about 5 to about 11 nt long) may be generated when an enzyme that has initiated synthesis aborts before completing the transcript and may then prime transcription of a complementary strand. Alternatively or additionally, RNA backfolding might lead to prolonged transcription, even generating extra-long (potentially twice the size, or even more) transcripts. Still further alternatively or additionally, re-initiation of transcription at certain open template structures might lead to transcription to generate an antisense strand. Reports have also suggested that template termination sites may influence production of dsRNA.

The present disclosure provides, among other things, technologies whereby dsRNA is reduced in RNA preparations. For example, in some embodiments, dsRNA present in an RNA preparation prepared according to the present disclosure is reduced relative to that present in an RNA preparation prepared, for example, using equimolar amounts of ATP, GTP, CTP and UTP.

Level of dsRNA, and thus of its reduction, can be determined using any of a variety of technologies including, without limitation, those discussed herein. For example, RNA can be spotted on a membrane such as a nylon membrane, blocked in appropriate buffer and detected using an antibody-based assay using antibodies specific for dsRNA, such as the J2 antibody (SCICONS English and Scientific Consulting), followed by staining with a secondary antibody anti-mouse HRP antibody (Jackson ImmunoResearch) (see EP 18 717 580.7). Antibody-based detection methods are well-known. RNA concentration can also be assessed using UV (e.g., Nanodrop) which may also indicate whether dsRNA concentration is present. RNA integrity can be assessed using a Bioanalyzer (Agilent).

The term “immunogenicity” refers to the ability of a particular substance, in particular RNA, to provoke an immune response in the body of an animal such as a human, e.g., either an innate immune response or an adaptive immune response, or both. In other words, immunogenicity is the ability to induce a humoral and/or cell mediated immune response. Unwanted immunogenicity includes an immune response by an organism against a therapeutic substance such as a drug. This reaction may inactivate the therapeutic effects of the treatment and may induce adverse effects. Without wishing to be bound by any particular theory, it is believed that immunogenicity of many RNA preparations, and particularly of those produced by conventional in vitro transcription reactions, is at least in part due to the content of dsRNA therein.

In some embodiments, RNA preparations described herein (e.g., which are produced by a method provided herein) are significantly less immunogenic than RNAs and compositions comprising RNA transcribed from the same DNA template using previously known methods, such as using equimolar amounts of adenosine triphosphate (ATP), guanosine triphosphate (GTP), cytidine triphosphate (CTP) and uridine triphosphate (UTP), or functional analogs thereof. In some embodiments, the RNA or composition comprising RNA of the invention (i.e., a provided RNA preparation) is at least 5% less immunogenic than an RNA or composition comprising RNA transcribed using equimolar amounts of ATP, GTP, CTP and UTP, or functional analogs thereof. In some embodiments, immunogenicity is reduced by at least 10%. In some embodiments, immunogenicity is reduced by at least 20%. In some embodiments, immunogenicity is reduced by at least 30%. In some embodiments, immunogenicity is reduced by at least 40%. In some embodiments, immunogenicity is reduced by at least 50%. In some embodiments immunogenicity is reduced by at least 60%. In some embodiments, immunogenicity is reduced by at least 70%. In some embodiments, immunogenicity is reduced by at least 80%. In some embodiments, immunogenicity is reduced by at least 90%. In some embodiments, immunogenicity is removed or essentially removed, i.e. reduced by about 100%.

In some embodiments, relative immunogenicity of RNA or a composition comprising RNA transcribed according to the invention (i.e., immunogenicity of a provided RNA preparation) and RNA or a composition comprising RNA transcribed using relevant control method known in the art, such as using equimolar amounts of ATP, GTP, CTP and UTP, or functional analogs thereof, may be determined by determining the quantity of the provided RNA preparation to elicit the same result to the same degree (e.g. expression of the same amount of protein) as a given quantity of the RNA or composition comprising RNA transcribed using the control method, e.g., using equimolar amounts of ATP, GTP, CTP and UTP, or functional analogs thereof. In an embodiment, the relative immunogenicity of the provided RNA preparation, and its respective counterpart transcribed using a relevant control method may be determined by determining the quantity of cytokine (e.g. IL-12, IFN-α, TNF-α, RANTES, MIP-1α or β, IL-6, IFN-β, or IL-8) secreted in response to administration of the RNA or composition comprising RNA transcribed according to the invention, relative to the same quantity of the RNA or composition comprising RNA transcribed using the control method. For example, if one-half as much cytokine is secreted, then the RNA or composition comprising RNA transcribed according to the invention is 50% less immunogenic than the RNA or composition comprising RNA transcribed using the appropriate control method.

“Significantly less immunogenic” refers to a detectable decrease in immunogenicity. In an embodiment, the term refers to a decrease such that an effective amount of the RNA or RNA comprising composition can be administered or repeatedly administered without triggering a detectable immune response. In some embodiments, the term refers to a decrease such that the RNA or RNA comprising composition can be repeatedly administered without eliciting an immune response sufficient to detectably reduce expression of the peptide or protein encoded by the RNA, e.g., comprised in the composition comprising RNA. In some embodiments, the decrease is such that the RNA or composition comprising RNA can be repeatedly administered without eliciting an immune response sufficient to eliminate expression of the peptide or protein encoded by the RNA.

As demonstrated herein, immunogenicity of an RNA or a composition comprising RNA (i.e., of an RNA preparation) can be decreased by transcribing the RNA using a method according to the invention as described herein. In some embodiments, in such methods the starting concentration of UTP or a functional analog thereof in the reaction mix used for transcribing the RNA from a template is lower than the starting concentration of CTP and/or ATP, or functional analogs thereof. In some embodiments, a method comprising transcribing RNA using a starting concentration of UTP or a functional analog thereof that is lower than the starting concentration of CTP and/or ATP, or a functional analog thereof, and supplementing the transcription reaction mix during the course of the transcription reaction with a composition comprising UTP, or a functional analog thereof, results in reduced formation of dsRNA during transcription as compared to an appropriate control transcription reaction, such as carrying out the method using equimolar amounts of ATP, GTP, CTP and UTP, or functional analogs thereof. Thus, in some embodiments, transcribing RNA according to a method of the invention results in RNA being less immunogenic as compared to RNA transcribed using equimolar amounts of ATP, GTP, CTP and UTP, or functional analogs thereof, as described herein. In some embodiments, transcribing RNA according to a method of the invention results in increased RNA yield as compared to RNA transcribed using equimolar amounts of ATP, GTP, CTP and UTP, or functional analogs thereof, as described herein.

### In Vitro Transcription Reactions

As described herein, RNA can be synthesized in vitro. Among other things, in vitro transcription permits use of cap-analogs (e.g., non-naturally occurring cap analogs) which may, for example, be added to the in vitro transcription reaction. Those skilled in the art will appreciate that, in many embodiments, a poly(A)-tail of an RNA molecule, if present, is encoded by a complementary sequence on the transcribed template (e.g, by a poly-(dT) sequence in a DNA template). Alternatively or additionally, in some embodiments, capping and/or poly(A)-tail addition can be achieved enzymatically after transcription, as is known in the art.

Those skilled in the art will appreciate that an in vitro transcription reaction typically includes: (1) a template (often DNA, typically linear) that comprises a promoter directing transcription of a sequence of interest; (2) ribonucleotide triphosphates (which may be natural compounds or analogs); (3) a buffer system (typically including magnesium ions); and (4) an RNA polymerase. Thus, according to the invention, an RNA transcription reaction typically comprises: (1) a template (e.g., DNA, typically linear) that may comprise a promoter directing transcription of a sequence of interest; (2) ribonucleotide triphosphates (natural or functional analogs thereof); (3) a buffer system such as the buffers described herein, e.g., selected according the RNA polymerase utilized; and (4) an RNA polymerase. In some embodiments, a transcription reaction mix may further comprise an Rnase inhibitor. In some embodiments, a transcription reaction mix may further comprise a pyrophosphatase (e.g., an inorganic pyrophosphatase). In some embodiments, a transcription reaction mix may further comprise one or more salts (e.g., monovalent salts and/or divalent salts such as salts comprising Li⁺, Na⁺, K⁺, NH⁴⁺, tris(hydroxymethyl)aminomethane cation, Mg²⁺, Ba²⁺ or Mn²⁺), a reducing agent (e.g., dithithreitol, 2-mercaptoethanol, etc.), spermidine, or combinations thereof. In some embodiments, certain reaction components are added in a specific order (e.g., pyrophosphatase and polymerase added last). In some embodiments, agitation rate is increased following the addition of specific reaction components (e.g., pyrophosphatase, polymerase).

Those skilled in the art will be aware that, particularly for large scale (e.g., that generate greater than about 50 ug, or more commonly greater than about 100 ug RNA per reaction, or that generate within a range of about 120 ug to about 180 ug per ug of template), it is desirable to reduce variation in the in vitro transcription reaction. For example, in many embodiments, it may be desirable to control the concentration of monovalent or divalent cations and/or the reaction temperature. In some embodiments, the cation in a reaction mix according to the invention is Li⁺, Na⁺, K⁺, NH⁴⁺, tris(hydroxymethyl)aminomethane cation, Mg²⁺, Ba²⁺ or Mn²⁺. For example, in many embodiments, it may be desirable to control concentration of Mg²⁺ and/or reaction temperature. In some embodiments, Mg²⁺ is reduced and/or temperature is increased (see, e.g., Mu et al., Nuc Acids Res. 10:5239).

In some embodiments, the present disclosure, among other things, provides technologies for large-scale manufacturing a pharmaceutical-grade composition or preparation comprising RNA, for example, at a mass batch throughput of at least 10 g RNA (including, e.g., at least 15 g RNA, at least 20 g RNA, at least 25 g RNA, at least 30 g RNA, at least 35 g RNA, at least 40 g RNA, at least 45 g RNA, at least 50 g RNA, at least 55 g RNA, at least 60 g RNA, at least 70 g RNA, at least 80 g RNA, at least 90 g RNA, at least 100 g RNA, at least 150 g RNA, at least 200 g RNA, or more). In some embodiments, such a method described herein can be used to produce a mass batch throughput of about 10 g to about 300 g RNA, about 10 g to about 200 g RNA, about 10 g to about 100 g RNA, about 30 g to about 60 g RNA, or about 50 g RNA to 300 g RNA. In some embodiments, such a method described herein is useful for large scale manufacturing that produces a mass batch throughput of at least 1 g RNA per hour or more, such as at least 1.5 g RNA per hour (including, e.g., at least 2 g RNA per hour, at least 2.5 g RNA per hour, at least 3 g RNA per hour, at least 3.5 g RNA per hour, at least 4 g RNA per hour, at least 4.5 g RNA per hour, at least 5 g RNA per hour, at least 5.5 g RNA per hour, at least 6 g RNA per hour, at least 6.5 g RNA per hour, at least 7 g RNA per hour, at least 7.5 g RNA per hour, at least 8 g RNA per hour, at least 8.5 g RNA per hour, at least 9 g RNA per hour, at least 10 g RNA per hour or higher). In some embodiments, large scale manufacture methods described herein can reach a capacity of 15 g RNA per hour to 20 g RNA per hour (e.g., about 17 g per hour).

Those skilled in the art will appreciate that the term “polymerase” generally refers to a molecular entity capable of catalyzing the synthesis of a polymeric molecule from monomeric building blocks. An “RNA polymerase” is a molecular entity capable of catalyzing the synthesis of a RNA molecule from ribonucleotide building blocks. A “RNA-dependent RNA polymerase” or “RdRP”, is an enzyme that catalyzes the transcription of RNA from an RNA template. A “DNA polymerase” is a molecular entity capable of catalyzing the synthesis of a DNA molecule from deoxy ribonucleotide building blocks. For the case of DNA polymerases and RNA polymerases, the molecular entity is typically a protein or an assembly or complex of multiple proteins. Typically, a DNA polymerase synthesizes a DNA molecule based on a template nucleic acid, which is typically a DNA molecule. Typically, a RNA polymerase synthesizes a RNA molecule based on a template nucleic acid, which is either a DNA molecule (in that case the RNA polymerase is a DNA-dependent RNA polymerase, DdRP), or is a RNA molecule (in that case the RNA polymerase is a RNA-dependent RNA polymerase, RdRP).

Various RNA polymerases that are suitable for transcription reactions are known in the art, including, but not limited to, DNA dependent RNA polymerases (e.g., T7 RNA polymerase, T3 RNA polymerase, SP6 RNA polymerase, N4 virion RNA polymerase, or a variant or functional domain thereof). Naturally catalysed RNA-dependent RNA polymerases are typically encoded by all RNA viruses except retroviruses. Typical representatives of viruses encoding a RNA-dependent RNA polymerase are alphaviruses. A skilled person will understand that an RNA polymerase utilized herein may be a recombinant RNA polymerase, and/or a purified RNA polymerase, i.e., not as part of a cell extract, which contains other components in addition to the RNA polymerases. In some embodiments, an RNA polymerase that is useful for commercial-scale transcription is a T7 RNA polymerase. In some embodiments, an inorganic pyrphosphatase may be added to improve the yield of transcription reaction (e.g., in some embodiments catalysed by T7 RNA polymerase).

Among other things, the present disclosure establishes that limiting nucleotides can reduce formation of dsRNA in in vitro reactions. The present disclosure specifically demonstrates that limitation of UTP in vitro transcription reactions, can reduce formation of dsRNA, and can be particularly useful for production of transcripts that may include a polyA sequence such as, for example, a polyA tail. Without wishing to be bound by theory, we propose that the observed reduction in dsRNA production may be attributable reduction of backwards transcription (e.g., initiated upon hybridization with a polyA sequence such as the polyA tail).

Those skilled in the art will appreciate that “limitation of UTP” as used herein means restriction on level of nucleotide that functionally pairs with an A residue in a template such that it is utilized in templated synthesis (i.e., in the in vitro transcription reaction) and incorporated into the produced strand; such a nucleotide is referred to herein as “UTP or a functional analog thereof”). In some embodiments, a functional analog is also translatable, typically as a “U”.

Thus, among other things, the present disclosure provides certain in vitro transcription technologies in which concentration of UTP (and/or functional analog(s) thereof) is limited, e.g., is lower than the concentration of CTP and/or ATP or functional analogs thereof. In some embodiments, UTP is limited at initiation of the reaction. In some embodiments, UTP is limited throughout the reaction.

For example, the present disclosure provides in vitro transcription reactions in which UTP is limited (e.g., concentration of UTP and functional analogs thereof is lower than that of one or more of the other nucleotides (i.e., adenosine triphosphate (ATP) and functional analogs thereof, guanosine triphosphate (GTP) and functional analogs thereof, and cytidine triphosphate (CTP) and functional analogs thereof).

Moreover, the present disclosure, among other things, provides technologies in which UTP is limited as described herein at the initial in vitro transcription reaction. In some embodiments, the present disclosure, among other things, provides technologies in which an initial in vitro transcription reaction with limitation of UTP is supplemented with UTP or functional analog thereof over time; in some such embodiments, such supplementing is by one or more discrete feeding events. In some embodiments, such supplementing may be performed by a continuous process, for example in some embodiments in which the rate of UTP supplementation is comparable to the rate of UTP consumption during the transcription reaction. In some embodiments, UTP is limited during supplementation (e.g., is supplemented at a concentration so that, after such supplementation, UTP or functional analog thereof is present in the reaction at a concentration lower than that of one or more, and in some embodiments, all of ATP or functional analog thereof, GTP or functional analog thereof, and/or CTP or functional analog thereof.

In some embodiments, an in vitro transcription reaction is supplemented with a plurality of nucleotides (e.g., with UTP or functional analog thereof and also with one or more other nucleotides—e.g., ATP or functional analog thereof, GTP or functional analog thereof, and/or CTP or functional analog thereof).

In some embodiments, both UTP and GTP are limited in the initial reaction and/or during supplementation.

### RNA Products

The present disclosure provides technologies useful for production of RNA products (i.e., for manufacturing preparations of particular RNA products).

In certain embodiments of the present disclosure, the produced RNA is a messenger RNA (mRNA), e.g., that relates to an RNA transcript which encodes a peptide or protein. As known in the art, in many instances, mRNA may contain a 5′ untranslated region (5′ UTR), a peptide coding region and a 3′ untranslated region (3′ UTR).

In some embodiments, an RNA product is produced by in vitro transcription. In some embodiments, an mRNA product is produced by in vitro transcription, for example using a DNA template (where DNA refers to a nucleic acid that contains deoxyribonucleotides). In some embodiments, RNA synthesis can also take place within cells or other systems.

In some embodiments, an RNA product is in vitro transcribed RNA (IVT-RNA) and may be obtained by in vitro transcription of an appropriate DNA template.

In certain embodiments, a DNA template is a linear molecule. In certain embodiments, a DNA template is a circular molecule. Generally, DNA can be obtained or generated using methods known in the art, including, e.g., gene synthesis, recombinant DNA technology, or a combination thereof. In some embodiments, a DNA template comprises a nucleotide sequence coding for a transcribed region of interest (e.g., coding for a RNA such as an RNA described herein) and a promoter sequence that is recognized by an RNA polymerase selected for use in in vitro transcription. A promoter for controlling transcription can be any promoter for any RNA polymerase. Various RNA polymerases are known in the art and exemplary polymerases are disclosed herein. A skilled artisan, reading the present disclosure, will readily understand that an RNA polymerase utilized herein may be a recombinant RNA polymerase, and/or a purified RNA polymerase, i.e., not as part of a cell extract, which contains other components in addition to the RNA polymerases. One skilled in the art will recognize an appropriate promoter sequence for the selected RNA polymerase. In some embodiments, a DNA template can comprise a promoter sequence for a T7 RNA polymerase. A DNA template for in vitro transcription may be obtained by cloning of a nucleic acid, such as cDNA, and introducing it into an appropriate vector for in vitro transcription. Techniques for introducing nucleic acid sequences into vectors is well known in the art, e.g., cold fusion cloning and others. The cDNA may be obtained by reverse transcription of RNA.

In some embodiments, an RNA amenable to technologies described herein is a single-stranded RNA (e.g., mRNA as described herein). In some embodiments, a single-stranded RNA is a non-coding RNA in that its nucleotide sequence does not include an open reading frame (or complement thereof). In some embodiments, a single-stranded RNA has a nucleotide sequence that encodes (or is the complement of a sequence that encodes) a polypeptide as described herein.

In some embodiments, technologies described herein may be particularly useful for synthesizing a single-stranded RNA having a length of at least 500 ribonucleotides (such as, e.g., at least 600 ribonucleotides, at least 700 ribonucleotides, at least 800 ribonucleotides, at least 900 ribonucleotides, at least 1000 ribonucleotides, at least 1250 ribonucleotides, at least 1500 ribonucleotides, at least 1750 ribonucleotides, at least 2000 ribonucleotides, at least 2500 ribonucleotides, at least 3000 ribonucleotides, at least 3500 ribonucleotides, at least 4000 ribonucleotides, at least 4500 ribonucleotides, at least 5000 ribonucleotides, at least 6000 ribonucleotides, at least 7000 ribonucleotides, at least 8000 ribonucleotides, at least 9000 ribonucleotides, at least 10000 ribonucleotides, at least 11000 ribonucleotides, at least 12000 ribonucleotides or longer). In some embodiments, technologies described herein may be particularly useful for synthesizing a single-stranded RNA having a length of about 1000 ribonucleotides to 5000 ribonucleotides.

In some embodiments, a relevant RNA includes a polypeptide-encoding portion. In some particular embodiments, such a portion may encode a polypeptide that is or comprises an antigen (or an epitope thereof), a cytokine, an enzyme, etc. In some embodiments, an encoded polypeptide may be or include one or more neoantigens or neoepitopes associated with a tumor. In some embodiments, an encoded polypeptide may be or include an antigen (or epitope thereof) of an infectious agent (e.g., a bacterium, fungus, virus, etc.). In certain embodiments, an encoded polypeptide may be a variant of a wild type polypeptide.

The present disclosure specifically exemplifies manufacture of RNAs encoding viral antigen(s) (and/or epitope(s) thereof), for example coronavirus antigen(s) and/or epitope(s). For example, in some embodiments, the present disclosure exemplifies production of a single-stranded RNA whose nucleotide sequence encodes a coronavirus polypeptide or a variant thereof. In some embodiments, a single-stranded RNA comprises a nucleotide sequence that encodes a prefusion coronavirus spike protein, e.g., as described in WO 2018081318, the entire contents of which are incorporated herein by reference for purposes described herein.

In some embodiments, a single-stranded RNA comprises a nucleotide sequence that encodes a SARS-CoV-2 polypeptide (including, e.g., a spike (S) protein, a nucleocapsid (N) protein, envelope (E) protein, and a membrane (M) protein) or an immunogenic fragment thereof. In some embodiments, a single-stranded RNA comprises a nucleotide sequence that encodes a SARS-CoV-2 S polypeptide or an immunogenic fragment thereof (e.g., a receptor binding domain of a S protein). In some embodiments, such a SARS-CoV-2 S polypeptide or an immunogenic fragment thereof may be a mutant protein. In some embodiments, an RNA for use in accordance with the present disclosure encodes a SARS-CoV-2 spike protein with K986P and V978P mutations.

In some embodiments, a single-stranded RNA (e.g., mRNA as described herein) may comprise a secretion signal-encoding region (e.g., a secretion signal-encoding region that allows an encoded target entity to be secreted upon translation by cells). In some embodiments, such a secretion signal-encoding region may be or comprise a non-human secretion signal. In some embodiments, such a secretion signal-encoding region may be or comprise a human secretion signal.

In some embodiments, a single-stranded RNA (e.g., mRNA as described herein) may comprise at least one non-coding sequence element (e.g., to enhance RNA stability and/or translation efficiency). Examples of non-coding sequence elements include but are not limited to a 3′ untranslated region (UTR), a 5′ UTR, a cap structure for co-transcriptional capping of mRNA, a poly adenine (polyA) tail, and any combinations thereof.

UTRs (5′ UTRs and/or 3′UTRs): In some embodiments, a single-stranded RNA can comprise a nucleotide sequence that encodes a 5′UTR of interest and/or a 3′ UTR of interest. One of skill in the art will appreciate that untranslated regions (e.g., 3′ UTR and/or 5′ UTR) of a mRNA sequence can contribute to mRNA stability, mRNA localization, and/or translational efficiency. In some embodiments, an untranslated region (UTR) can be present 5′ (upstream) of an open reading frame (5′ UTR) and/or 3′ (downstream) of an open reading frame (3′ UTR).

In some embodiments, a single-stranded RNA can comprise a 5′ UTR nucleotide sequence and/or a 3′ UTR nucleotide sequence. In some embodiments, such a 5′ UTR sequence can be operably linked to a 3′ of a coding sequence (e.g., encompassing one or more coding regions). Additionally or alternatively, in some embodiments, a 3′ UTR sequence can be operably linked to 5′ of a coding sequence (e.g., encompassing one or more coding regions). In some embodiments, 5′- and/or 3′-untranslated regions may, according to the invention, be functionally linked to an open reading frame, so as for these regions to be associated with the open reading frame in such a way that the stability and/or translation efficiency of the RNA comprising said open reading frame are increased.

In some embodiments, 5′ and 3′ UTR sequences included in a single-stranded RNA can consist of or comprise naturally occurring or endogenous 5′ and 3′ UTR sequences for an open reading frame of a gene of interest. Alternatively, in some embodiments, 5′ and/or 3′ UTR sequences included in a single-stranded RNA are not endogenous to a coding sequence (e.g., encompassing one or more coding regions); in some such embodiments, such 5′ and/or 3′ UTR sequences can be useful for modifying the stability and/or translation efficiency of an RNA sequence transcribed. For example, a skilled artisan will appreciate that AU-rich elements in 3′ UTR sequences can decrease the stability of mRNA. Therefore, as will be understood by a skilled artisan, 3′ and/or 5′ UTRs can be selected or designed to increase the stability of the transcribed RNA based on properties of UTRs that are well known in the art.

For example, one skilled in the art will appreciate that, in some embodiments, a nucleotide sequence consisting of or comprising a Kozak sequence of an open reading frame sequence of a gene or nucleotide sequence of interest can be selected and used as a nucleotide sequence encoding a 5′ UTR. As will be understood by a skilled artisan, Kozak sequences are known to increase the efficiency of translation of some RNA transcripts, but are not necessarily required for all RNAs to enable efficient translation. In some embodiments, a single-stranded RNA can comprise a nucleotide sequence that encodes a 5′ UTR derived from an RNA virus whose RNA genome is stable in cells. In some embodiments, various modified ribonucleotides (e.g., as described herein) can be used in the 3′ and/or 5′ UTRs, for example, to impede exonuclease degradation of the transcribed RNA sequence.

As those skilled in the art are aware, the Kozak sequence is a sequence initially described by Kozak, (1987), Nucleic Acids Res., 15: 8125-8148. The Kozak sequence on an RNA molecule such as an mRNA molecule is recognized by the ribosome as the translational start site. Typically, the Kozak sequence comprises an AUG start codon, immediately followed by a highly conserved G nucleotide: AUGG. In particular, it was described that a Kozak sequence may be identified by (gcc)gccRccAUGG, as follows: (i) lower case letters denote the most common base at a position where the base can nevertheless vary; (ii) upper case letters indicate highly conserved bases (e.g. ‘AUGG’); (iii) ‘R’ indicates a purine (adenine or guanine); (iv) the sequence in brackets ((gcc)) is of uncertain significance; (v) the underlined AUG base triplet represents the start codon.

In some embodiments, an RNA produced according to the present invention comprises
(1) a 5′ UTR,
(2) an open reading frame, e.g., encoding a peptide or protein of interest, and/or
(3) a 3′ UTR.

In some embodiments, such a 5′ UTR sequence can be operably linked to a 3′ of a coding sequence (e.g., encompassing one or more coding regions). Additionally or alternatively, in some embodiments, a 3′ UTR sequence can be operably linked to 5′ of a coding sequence (e.g., encompassing one or more coding regions).

In some embodiments, a 5′ UTR included in a single-stranded RNA may be derived from human α-globin mRNA combined with Kozak region.

In some embodiments, a single-stranded RNA may comprise one or more 3′UTRs. For example, in some embodiments, a single-stranded RNA may comprise two copies of 3′-UTRs derived from a globin mRNA, such as, e.g., alpha2-globin, alpha1-globin, beta-globin (e.g., a human beta-globin) mRNA. In some embodiments, two copies of 3′UTR derived from a human beta-globin mRNA may be used, e.g., in some embodiments which may be placed between a coding sequence of a single-stranded RNA and a poly(A)-tail, to improve protein expression levels and/or prolonged persistence of an mRNA. In some embodiments, a 3′ UTR included in a single-stranded RNA may be or comprise one or more (e.g., 1, 2, 3, or more) of the 3′UTR sequences disclosed in WO 2017/060314, the entire content of which is incorporated herein by reference for the purposes described herein. In some embodiments, a 3′-UTR may be a combination of at least two sequence elements (FI element) derived from the “amino terminal enhancer of split” (AES) mRNA (called F) and the mitochondrial encoded 12S ribosomal RNA (called I). These were identified by an ex vivo selection process for sequences that confer RNA stability and augment total protein expression (see WO 2017/060314, herein incorporated by reference).

The human beta-globin 3′ UTR, particularly two consecutive identical copies of the human beta-globin 3′ UTR, contributes to high transcript stability and translational efficiency (Holtkamp et al., (2006), Blood, 108: 4009-4017). Thus, in some embodiments, the replicase construct according to the present invention comprises two consecutive identical copies of the human beta-globin 3′ UTR. Thus, it comprises in the 5′->3′ direction: (a) optionally a 5′ UTR; (b) an open reading frame; (c) a 3′ UTR; said 3′ UTR comprising two consecutive identical copies of the human beta-globin 3′ UTR, a fragment thereof, or a variant of the human beta-globin 3′ UTR or fragment thereof.

In some embodiments, the RNA according to the present invention comprises a 3′ UTR which is active in order to increase translation efficiency and/or stability, but which is not the human beta-globin 3′ UTR, a fragment thereof, or a variant of the human beta-globin 3′ UTR or fragment thereof.

PolyA tail: In some embodiments, a single-stranded RNA can comprise a polyA tail. A polyA tail is a nucleotide sequence comprising a series of adenosine nucleotides, which can vary in length (e.g., at least 5 adenine nucleotides) and can be up to several hundred adenosine nucleotides. A polyA tail may be transcribed from a template, e.g., a DNA template, or may be added enzymatically after the transcription reaction. In some embodiments, a polyA tail is a nucleotide sequence comprising at least 30 adenosine nucleotides or more, including, e.g., at least 35, at least 40, at least 45, at least 50, at least 55, at least 60, at least 65, at least 70, at least 75, at least 80, at least 85, at least 90, at least 95, at least 100, or more adenosine nucleotides. In some embodiments, a polyA tail is or comprises a polyA homopolymeric tail. In some embodiments, a polyA tail may comprise one or more modified adenosine nucleosides, including, but not limited to, cordycepin and 8-azaadenosine. In some embodiments, a polyA tail may comprise one or more non-adenosine nucleotides. In some embodiments, a polyA tail may be or comprise a disrupted or modified polyA tail as described in WO 2016/005324, the entire content of which is incorporated herein by reference for the purpose described herein. For example, in some embodiments, a polyA tail included in a single-stranded RNA described herein may be or comprise a modified polyA sequence comprising: a linker sequence; a first sequence of at least 20 consecutive A nucleotides, which is 5′ of the linker sequence; and a second sequence of at least 20 consecutive A nucleotides, which is 3′ of the linker sequence. In some embodiments, a modified polyA sequence may comprise: a linker sequence comprising at least ten nucleotides (e.g., U, G, and/or C nucleotides); a first sequence of at least 30 consecutive A nucleotides, which is 5′ of the linker sequence; and a second sequence of at least 70 consecutive A nucleotides, which is 3′ of the linker sequence.

In some embodiments, a poly(A)-tail comprises or essentially consists of or consists of at least 20, in some embodiments at least 26, in some embodiments at least 40, in some embodiments at least 80, in some embodiments at least 100 and in some embodiments up to 500, in some embodiments up to 400, in some embodiments up to 300, in some embodiments up to 200, in some embodiments up to 150 and, in particular, about 120 A nucleotides. In some embodiments, a poly(A)-tail is a nucleotide sequence comprising at least 30 adenosine nucleotides or more, including, e.g., at least 35, at least 40, at least 45, at least 50, at least 55, at least 60, at least 65, at least 70, at least 75, at least 80, at least 85, at least 90, at least 95, at least 100, or more adenosine nucleotides. In this context “essentially consists of” means that most nucleotides in the poly(A)-tail, typically at least 50%, and in some embodiments at least 75% by number of nucleotides in the “poly(A)-tail”, are A nucleotides (adenylate), but permits that remaining nucleotides are nucleotides other than A nucleotides, such as U nucleotides (uridylate), G nucleotides (guanylate), C nucleotides (cytidylate). In this context “consists of” means that all nucleotides in the poly(A)-tail, i.e. 100% by number of nucleotides in the poly(A)-tail, are A nucleotides. The term “A nucleotide” or “A” refers to adenylate.

Indeed, it has been demonstrated that a 3′ poly(A)-tail of about 120 A nucleotides has a beneficial influence on the levels of RNA in transfected eukaryotic cells, as well as on the levels of protein that is translated from an open reading frame that is present upstream (5′) of the 3′ poly(A)-tail (Holtkamp et al., (2006), Blood, 108: 4009-4017).

According to the present invention, in some embodiments, a 3′ poly(A)-tail is attached during RNA transcription, i.e., during preparation of in vitro transcribed RNA, based on a DNA template comprising repeated dT nucleotides (deoxythymidylate) in the strand complementary to the coding strand. The DNA sequence encoding a poly(A)-tail (coding strand) is referred to as poly(A) cassette.

5′ cap: In some embodiments, an RNA product (e.g., a single-stranded RNA) prepared as described herein may comprise a 5′ cap, which may be incorporated into such a single-stranded RNA during transcription, or joined to such RNA post-transcription. In some embodiments, an RNA may comprise a 5′ cap structure for co-transcriptional capping of mRNA. Examples of a cap structure for co-transcriptional capping are known in the art, including, e.g., as described in WO 2017/053297, the entire content of which is incorporated herein by reference for the purposes described herein. In some embodiments, a 5′ cap included in an RNA product described herein is or comprises a cap1 structure. For example, in some embodiments, a cap1 structure may be or comprise m7G(5′)ppp(5′)(2′OMeA)pG, also known as m₂^{7,3′-O}Gppp(m₁^{2′-O})ApG.

In some embodiments, an RNA product (e.g., a single-stranded RNA) produced as described herein may comprise at least one modified ribonucleotide, for example, in some embodiments to increase the stability of such a RNA product and/or to decrease cytotoxicity of such RNA product. For example, in some embodiments, at least one of A, U, C, and G ribonucleotide of a single-stranded RNA may be replaced by a modified ribonucleotide. For example, in some embodiments, some or all of cytidine residues present in a single-stranded RNA may be replaced by a modified cytidine, which in some embodiments may be, e.g., 5-methyleytidine. Alternatively or additionally, in some embodiments, some or all of uridine residues present in a single-stranded RNA may be replaced by a modified uridine, which in some embodiments may be, e.g., pseudouridine, such as, e.g., 1-methylpseudouridine. In some embodiments, all uridine residues present in a single-stranded RNA is replaced by pseudouridine, e.g., 1-methylpseudouridine.

### DNA Templates

One of ordinary skill in the art will understand that a DNA template is typically used to direct synthesis of RNA (e.g., single-stranded RNA). In some embodiments, a DNA template is a linear DNA molecule. In some embodiments, a DNA template is a circular DNA molecule. DNA can be obtained or generated using methods known in the art, including, e.g., gene synthesis, recombinant DNA technology, or a combination thereof. In some embodiments, a DNA template comprises a nucleotide sequence coding for a transcribed region of interest (e.g., coding for a RNA described herein) and a promoter sequence that is recognized by an RNA polymerase selected for use in in vitro transcription. Various RNA polymerases are known in the art, including, e.g., DNA dependent RNA polymerases (e.g., a T7 RNA polymerase, a T3 RNA polymerase, a SP6 RNA polymerase, a N4 virion RNA polymerase, or a variant or functional domain thereof). A skilled artisan will readily understand that an RNA polymerase utilized herein may be a recombinant RNA polymerase, and/or a purified RNA polymerase, i.e., not as part of a cell extract, which contains other components in addition to the RNA polymerases. One skilled in the art will recognize an appropriate promoter sequence for the selected RNA polymerase. In some embodiments, a DNA template can comprise a promoter sequence for a T7 RNA polymerase.

In some embodiments, a DNA template comprises a nucleotide sequence coding for an RNA described herein (e.g., comprising a nucleotide sequence coding for an antigen of interest and optionally comprising one or more nucleotide sequences coding for characteristic elements of an RNA described herein, including, e.g., polyA tail, 3′ UTR, and/or 5′ UTR, etc.). In some embodiments, such a coding sequence may be generated by gene synthesis. In some embodiments, such a coding sequence may be inserted into a vector by cold fusion cloning.

In some embodiments, a DNA template may further comprise one or more of a recognition sequence for an appropriate restriction endonuclease (e.g., utilized for linearization), an appropriate resistance gene, and/or an appropriate origin of replication. In some embodiments, a DNA template may further comprise a recognition sequence for an appropriate restriction endonuclease (e.g., utilized for linearization such as, e.g., but not limited to a Class II restriction endonuclease), an appropriate resistance gene (e.g., but not limited to a kanamycin resistance gene), and an appropriate origin of replication.

In some embodiments, a DNA template may be or have been amplified, for example via polymerase chain reaction (PCR) from a plasmid DNA. In some embodiments, a plasmid DNA may be obtained, e.g., from bacterial cells (e.g., Escherichia coli (E. coli)) followed by an endotoxin- and animal product-free plasmid isolation procedure.

### Ribonucleotides

Ribonucleotides for use in in vitro transcription may include at least two or more (e.g., at least three or more, at least four or more, at least five or more, at least six or more) different types of ribonucleotides, each type having a different nucleoside. Ribonucleotides for use in in vitro transcription can include unmodified and/or modified ribonucleotides. Unmodified ribonucleotides include the purine bases adenine (A) and guanine (G), and the pyrimidine bases cytosine (C) and uracil (U). In some embodiments, all four types of unmodified ribonucleotides may be used for in vitro transcription.

In some embodiments, at least one type of ribonucleotide included in in vitro transcription is a modified ribonucleotide. Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. To the extent that such modifications interfere with translation (e.g., results in a reduction of 50% or more in translation relative to the absence of the modification—e.g., as characterized using a rabbit reticulocyte in vitro translation assay), such modified ribonucleotides, in some embodiments, are not desirable for use in technologies described herein.

In some embodiments, one or more modified nucleosides may be utilized, such as one or more functional analogs of one or more of adenosine triphosphate (ATP), guanosine triphosphate (GTP), cytidine triphosphate (CTP) and uridine triphosphate (UTP). When functional analogs of any one of ATP, GTP, CTP or UTP are used in the methods according to the invention, the resulting RNA molecule will comprise these functional analogs in place of ATP, GTP, CTP or UTP, respectively.

In some embodiments, a modified ribonucleotide may have at least one nucleoside (“base”) modification or substitution. Various nucleoside modifications or substitutions are known in the art; one of skill in the art will appreciate that modified nucleosides include, for example, but not limited to synthetic and natural nucleobases such as inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N6-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N6-(isopentyl)adenine, N6-(methyl) adenine, N6, N6-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N4-(acetyl)cytosine, 3-(3 amino-3 carboxypropyl)uracil, 2-(thio)uracil, 5-(methyl)-2-(thio)uracil, 5-(methylaminomethyl)-2 (thio)uracil, 4-(thio)uracil, 5-(methyl)-4 (thio)uracil, 5-(methylaminomethyl)-4 (thio)uracil, 5-(methyl)-2,4-(dithio)uracil, 5-(methylaminomethyl)-2,4 (dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N3-(methyl)uracil, 5-uracil (i.e., pseudouracil), 2-(thio)pseudouracil, 4-(thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4 (thio)pseudouracil, 5-(methyl)-4 (thio)pseudouracil, 5-(alkyl)-2,4 (dithio)pseudouracil, 5-(methyl)-2,4 (dithio)pseudouracil, 1-substituted pseudouracil (e.g., 1-methyl-pseudouridine), C-5 propynyl-uridine, 2-aminoadenosine, C5-bromouridine, C5-fluorouridine, C5-iodouridine, C5-propynyl-uridine, 1-substituted-2(thio)-pseudouracil, 1-substituted 4 (thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1 (aminocarbonylethylenyl)-4 (thio)pseudouracil, 1-(aminocarbonylethylenyl)-2,4-(dithio)pseudouracil, 1-(aminoalkylaminocarbonylethylenyl)-pseudouracil, 1 (aminoalkylamino-carbonylethylenyl)-2(thio)-pseudouracil, 1-(arninoalkylaninocarbonylethylenyl)-4 (thio)pseudouracil, 1-(arninoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, -(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5 nitroindole, 3 nitropyrrole, 6-(aza)pyrimidine, 2 (amino)purine, 2,6-(diamino)purine, 5 substituted pyrimidines, N2-substituted purines, N6-substituted purines, 06-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof.

In some embodiments, a modified nucleotide utilized in IVT systems and/or methods described herein may disrupt binding of an RNA to one or more mammalian (e.g., human) endogenous RNA sensors (e.g., innate immune RNA sensors), including, e.g., but not limited to toll-like receptor (TLR)3, TLR7, TLR8, retinoic acid-inducible gene I (RIG-I), melanoma differentiation-associated gene 5 (MDA5), protein kinase R (PKR), 2′-5′ oligoadenylate synthetase (OAS), and laboratory of genetics and physiology 2 (LGP2), and combinations thereof. In some embodiments, such modified ribonucleotides may include modifications as described in U.S. Pat. No. 9,334,328, the contents of which are incorporated herein by reference in their entireties for the purposes described herein. Modified nucleosides are typically desirable to be translatable in a host cell (e.g., presence of a modified nucleoside does not prevent translation of an RNA sequence into a respective protein sequence). Effects of modified nucleotides on translation can be assayed, by one of ordinary skill in the art using, for example, a rabbit reticulocyte lysate translation assay.

In some embodiments, a modified ribonucleotide may include a modified internucleoside linkage. Various such modified internucleoside linkages are known in the art; one of skill in the art will appreciate that non-limiting examples of modified internucleoside linkages that may be used in technologies provided herein include phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified internucleoside linkages that do not include a phosphorus atom therein may have internucleoside linkages that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thiofonnacetyl backbones; alkene containing backbones; sulfamate backbones; methyleneimino and methylenehydrazino backbones; sulfonate and sulfonamide backbones; amide backbones; and others having mixed N, O, S and CH2 component parts.

In some embodiments, a modified ribonucleotide may include one or more substituted sugar moieties. Various such modified sugar moieties are known in the art; one of skill in the art will appreciate that, in some embodiments, a sugar moiety of a ribonucleotide may include one of the following at the 2′ position: H (deoxyribose); OH (ribose); F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted. In some embodiments, a sugar moiety of a ribonucleotide may include a 2′ methoxyethoxy (2′-O—CH₂CH₂OCH₃, also known as 2′-O-(2-methoxyethyl) or 2′-MOE), 2′-dimethylaminooxyethoxy, i.e., a O(CH2)2ON(CH3)2 group, also known as 2′-DMAOE, and 2′-dimethylaminoethoxyethoxy (also known in the art as 2′O-dimethylaminoethoxyethyl or 2′-DMAEOE), i.e., 2′-O—CH₂—O—CH₂—N(CH₂)₂; 2′-methoxy (2′-OCH₃), 2′-aminopropoxy (2′-OCH₂CH₂CH₂NH₂) and 2′-fluoro (2′-F). Similar modifications can also be made at other positions, for example, at the 3′ position of the sugar on the 3′ terminal nucleotide or in 2′-5′ linked nucleotides and the 5′ position of 5′ terminal nucleotide.

In some embodiments, a mixture of ribonucleotides that are useful for an in vitro transcription reaction may comprise UTP or a functional thereof in combination with at least one or all of ATP or a functional analog thereof, CTP or a functional analog thereof, and GTP or a functional analog thereof. In some embodiments, a functional analog of UTP is or comprises N1-methylpseudouridine-5′ triphosphate (m1ΨTP). As described herein, the present disclosure, among other things, provides technologies in which UTP is limited as described herein at the initial in vitro transcription reaction (e.g., at a concentration lower than that of one or more, and in some embodiments all of ATP, or functional analog thereof, GTP, or functional analog thereof, and/or CTP or functional analog thereof. In some embodiments, the present disclosure, among other things, provides technologies in which an initial in vitro transcription reaction with limitation of UTP is supplemented with UTP or functional analog thereof over time; in some such embodiments, such supplementing is by one or more discrete feeding events. In some embodiments, such supplementing may be performed by a continuous process, for example in some embodiments in which the rate of UTP supplementation is comparable to the rate of UTP consumption during the transcription reaction. In some embodiments, UTP is limited during supplementation (e.g., is supplemented at a concentration so that, after such supplementation, UTP or functional analog thereof is present in the reaction at a concentration lower than that of one or more, and in some embodiments, all of ATP or functional analog thereof, GTP or functional analog thereof, and/or CTP or functional analog thereof.

In some embodiments, a functional analog of a nucleoside triphosphate (NTP) comprises a modified nucleoside. In some embodiments, a modified nucleoside is a modified uridine. In certain embodiments, replacing uridine by a modified nucleoside is done by replacing UTP with a functional analog thereof. In some embodiments, a functional analog of a UTP is a triphosphate of a modified uridine nucleoside.

In some embodiments, a modified uridine nucleoside is independently selected from pseudouridine (ψ), N1-methyl-pseudouridine (m1ψ), and 5-methyl-uridine (m5U). In some embodiments, the modified nucleoside comprises pseudouridine (ψ). In some embodiments, the modified nucleoside comprises N1-methyl-pseudouridine (m1ψ). In some embodiments, a modified nucleoside comprises 5-methyl-uridine (m5U).

In some embodiments, RNA may comprise more than one type of modified uridine nucleoside. In some embodiments, RNA comprises more than one type of modified uridine nucleoside independently selected from pseudouridine (ψ), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (ψ) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ), N1-methyl-pseudouridine (m1ψ), and 5-methyl-uridine (m5U).

In some embodiments, a modified nucleoside is any one or more selected from the group consisting of 3-methyl-uridine (m3U), 5-methoxy-uridine (mo5U), 5-aza-uridine, 6-aza-uridine, 2-thio-5-aza-uridine, 2-thio-uridine (s2U), 4-thio-uridine (s4U), 3-methyluridine, 4-thio-pseudouridine, 2-thio-pseudouridine, 5-hydroxy-uridine (ho5U), 5-aminoallyl-uridine, 5-halo-uridine (e.g., 5-iodo-uridine or 5-bromo-uridine), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), I-ethyl-pseudouridine, 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmmn5s2U), 5-propynyl-uridine, 1-propynyl-pseudouridine, 5-taurinomethyl-uridine (τm5U), 1-taurinomethyl-pseudouridine, 5-taurinomethyl-2-thio-uridine (τm5s2U), 1-taurinomethyl-4-thio-pseudouridine), 5-methyl-2-thio-uridine (m5s2U), 1-methyl-4-thio-pseudouridine (m1s4ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3 ψ), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-0-dimethyl-uridine (m3Um), 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

In some embodiments, a functional analog of an NTP comprises a modified nucleoside. In some embodiments, the modified nucleoside is a modified adenosine. In certain embodiments, replacing adenosine by a modified nucleoside is done by replacing ATP with a functional analog thereof. In some embodiments, a functional analog of an ATP is a triphosphate of a modified adenosine nucleoside.

In some embodiments, a modified nucleoside is any one or more selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N1-methyl-adenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, α-thio-adenosine, 8-azido-adenosine, 7-deaza-adenosine, 7-methyladenine, 2-methylthio-adenine, and 2-methoxy-adenine.

In some embodiments, a functional analog of an NTP comprises a modified nucleoside. In some embodiments, the modified nucleoside is a modified guanosine. In certain embodiments, replacing guanosine by a modified nucleoside is done by replacing GTP with a functional analog thereof. In some embodiments, a functional analog of a GTP is a triphosphate of a modified guanosine nucleoside.

In some embodiments, a modified nucleoside is any one or more selected from the group consisting of 1-methyl-inosine, wyosine, wybutosine, α-thio-guanosine, 6-methyl-guanosine, 7-deazaguanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 06-methyl-guanosine, N1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some embodiments, a functional analog of an NTP comprises a modified nucleoside. In some embodiments, the modified nucleoside is a modified cytidine. In certain embodiments, replacing cytidine by a modified nucleoside is done by replacing CTP with a functional analog thereof. In some embodiments, a functional analog of a CTP is a triphosphate of a modified cytidine nucleoside.

In some embodiments, a modified nucleoside is any one or more selected from the group consisting of 5-aza-cytidine, 6-azacytidine, α-thio-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methylpseudoisocytidine, 4-thio-1-methyl-1-deaza-pseudoisocytidine, 1-methyl-1-deaza-pseudoisocytidine, zebularine, 5-azazebularine, 5-methyl-zebularine, 5-aza-2-thio-zebularine, 2-thio-zebularine, 2-methoxy-cytidine, 2-methoxy-5-methylcytidine, 4-methoxy-pseudoisocytidine, and 4-methoxy-1-methyl-pseudoisocytidine.

### 5′ Cap

In some embodiments, an RNA produced by technologies described herein may comprise a cap at its 5′ end.

The term “non-extending nucleotide” or “start nucleotide” or similar terms mean a nucleotide that does not have a 5′ triphosphate or has a 5′ triphosphate that has been modified, such that the nucleotide can be incorporated only at the 5′ end of a transcript, and that has a 3′ hydroxy, so it can be extended at the 3′ position. A start nucleotide is a nucleotide which corresponds to the first nucleotide of an RNA. In some embodiments, addition of the start nucleotide increases the initiation rate of an RNA polymerase. In certain embodiments, the start nucleotide is or comprises a nucleoside monophosphate, a nucleoside diphosphate, a nucleoside triphosphate or a dinucleoside triphosphate. In case the first nucleotide of an RNA is G, the start nucleotide may be GTP or GMP or a functional analog thereof as described herein. In some embodiments, a start nucleotide is a dinucleotide or a trinucleotide. In some embodiments, a start nucleotide is a nucleoside-5′-triphosphate. In some embodiments, the first nucleotide of the RNA is G, the start nucleotide is a cap analog of G and the corresponding ribonucleoside triphosphate is GTP. In some embodiments, a start nucleotide is a naturally occurring 5′ cap or 5′ cap analog such as a cap analog described herein. In some embodiments, a cap is or comprises a guanine nucleotide. These nucleotides may or may not have cap functionality. Start nucleotides include 5′ caps and 5′ cap analogs such as those described herein.

In some embodiments, an RNA produced according to technologies provided herein (i.e., an RNA according to the invention) has an initial nucleotide (start nucleotide) that is not GTP. In some embodiments, an RNA according to the invention has a start nucleotide that competes with GTP or a functional analog thereof for incorporation into the RNA. In some embodiments, a start nucleotide is incorporated into the RNA as readily as any other nucleotide. In some embodiments, a start nucleotide is incorporated into the RNA more efficiently than any other nucleotide, in particular more efficiently than GTP or a functional analog thereof. In some embodiments, a start nucleotide is incorporated into the RNA less efficiently than any other nucleotide, in particular less efficiently than GTP or a functional analog thereof. In some embodiments, a start nucleotide is supplemented during the course of transcription. In some embodiments, a start nucleotide is added to the reaction mix before the start of the transcription reaction.

In some embodiments, in the reaction mix used according to the invention, the start nucleotide corresponding to the first nucleotide of an RNA molecule to be produced is added in excess compared to the fraction of the nucleotide predicted to be found or found at the first position of the RNA molecule. In some embodiments, in the reaction mix, the start nucleotide corresponding to the first nucleotide of an RNA molecule to be produced is added in excess compared to the fraction of the nucleotide with which it competes for incorporation into the RNA molecule. For example, in some embodiments, the first nucleotide is G and a 5′ cap or 5′ cap analog is present in excess over GTP in the initial reaction mix. In some embodiments the start nucleotide is added with an initial concentration in the range of about 1 to 20 mM, 1 to 17.5 mM, 1 to 15 mM, 1 to 12.5 mM, 1 to 10 mM, 1 to 7.5 mM, 1 to 5 mM or 1 to 2.5 mM. In some embodiments the start nucleotide is added, compared to the nucleotide with which it competes for incorporation into the RNA in excess of at least about 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1 or even more. For example, in some embodiments, the starting concentration of a 5′ cap or 5′ cap analog to the starting concentration of GTP is between about 2:1 and about 20:1, such as about 2:1, 3:1, in some embodiments 4:1, in some embodiments 5:1, in some embodiments 6:1, more in some embodiments 7:1, 8:1, 9:1, 10:1 or even higher.

The terms “5′ cap”, “cap”, “5′ cap structure”, “cap structure”, “cap nucleotide” and “5′ cap nucleotide” are used synonymously to refer to a dinucleotide that is found on the 5′ end of some nucleic acids such as mRNA. A 5′ cap is a structure wherein a (optionally modified) guanosine is bonded to the first nucleotide of an mRNA molecule via a 5′ to 5′ triphosphate linkage (or modified triphosphate linkage in the case of certain cap analogs). In some embodiments, this guanosine is methylated at the 7-position (e.g., naturally occurring m7G cap). The term “conventional 5′ cap” refers to a naturally occurring RNA 5′ cap, in some embodiments to the 7-methylguanosine cap (m7G). Providing an RNA with a 5′ cap or 5′ cap analog may be achieved by in vitro transcription, in which the 5′ cap is co-transcriptionally incorporated into the RNA strand (transcription and capping reaction), or may be attached to RNA post-transcriptionally, e.g., after in vitro transcribing the RNA, using capping enzymes such as capping enzymes from vaccinia virus or Saccharomyces cerevisiae capping enzyme system. Alternatively, a capped RNA can be obtained by in vitro transcription (IVT) of a DNA template, wherein, in addition to the GTP, an IVT system also contains a 5′ cap or 5′ cap analog, e.g., as known in the art and described herein. Methods to provide an RNA with a 5′ cap are well known in the art. In capped RNA, the 3′ position of the first base of a (capped) RNA molecule is linked to the 5′ position of the subsequent base of the RNA molecule (“second base”) via a phosphodiester bond.

Those skilled in the art will appreciate that, in some embodiments, addition of a 5′ cap to an RNA (e.g., mRNA) can facilitate recognition and attachment of the RNA to a ribosome to initiate translation and enhances translation efficiency. Those skilled in the art will also appreciate that a 5′ cap can also protect an RNA product from 5′ exonuclease mediated degradation and thus increases half-life.

As noted above, in some embodiments, an RNA produced by technologies described herein may comprise a cap at its 5′ end. In some embodiments, the RNA does not have uncapped 5′-triphosphates. In some embodiments, the RNA may be modified by a 5′ cap analog. In some embodiments, a 5′ cap is or comprises a synthetic 5′ cap analog that resembles an RNA 5′ cap structure and possesses the ability to stabilize RNA if attached thereto, including, e.g., but not limited to anti-reverse cap analogs (ARCAs) which are known in the art and are described herein. Those skilled in the art will appreciate that addition of a 5′ cap to an RNA (e.g., mRNA) can facilitate recognition and attachment of the RNA to a ribosome to initiate translation and enhances translation efficiency. Those skilled in the art will also appreciate that a 5′ cap can also protect an RNA product from 5′ exonuclease mediated degradation and thus increases half-life. Methods for capping are known in the art; one of ordinary skill in the art will appreciate that in some embodiments, capping may be performed after in vitro transcription in the presence of a capping system (e.g., an enzyme-based capping system such as, e.g., capping enzymes of vaccinia virus). In some embodiments, a capped RNA may be obtained by in vitro capping of RNA that has a 5′ triphosphate group or RNA that has a 5′ diphosphate group with a capping enzyme system (including, e.g., but not limited to vaccinia capping enzyme system or Saccharomyces cerevisiae capping enzyme system). In some embodiments, a capping agent may be introduced into an in vitro transcription reaction mixture (e.g., ones as described herein), along with a plurality of ribonucleotides such that a cap is incorporated into an RNA during transcription (also known as co-transcriptional capping). While it may be desirable to include, in some embodiments, a 5′ cap in an RNA, an RNA, in some embodiments, may not have a 5′ cap.

The most frequently used method to make capped RNAs in vitro is by transcribing a DNA template with an RNAP such as a bacterial or bacteriophage RNA polymerase in the presence of all four ribonucleoside triphosphates and a 5′ cap or 5′ cap analog, such as m7G(5′)ppp(5′)G (also called m7GpppG). The RNA polymerase initiates transcription with a nucleophilic attack by the 3′-OH of the guanosine moiety of m7GpppG on the α-phosphate of the next templated nucleoside triphosphate (pppN), resulting in the intermediate m7GpppGpN (wherein N is the second base of the RNA molecule). The formation of the competing GTP-initiated product pppGpN is suppressed by adding the 5′ cap or 5′ cap analog in excess over GTP as described herein.

In some embodiments, a 5′ capping agent can be added to an in vitro transcription reaction mixture. In some embodiments, a 5′ capping agent may comprise a modified nucleotide, for example, a modified guanine nucleotide. In some embodiments, a 5′ capping agent may comprise, for example, a methyl group or groups, glyceryl, inverted deoxy abasic moiety, 4′5′ methylene nucleotide, l-(beta-D-erythrofuranosyl) nucleotide, 4′ thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety, inosine, N1-methyl-guanosine, 2′-fluoro-guanosine, 7′deaza-guanosine, 8-oxo-guanosine, 2-amino-guanosine, LNA-guanosine, 2-azido-guanosine. In some embodiments, a 5′ capping agent may be or comprise a dinucleotide cap analog (including, e.g., a m7GpppG cap analog or an N7-methyl, 2′-O-methyl-GpppG anti-reverse cap analog (ARCA) cap analog or an N7-methyl, 3′-O-methyl-GpppG ARCA cap analog). In some embodiments, a 5′ capping agent comprises a 5′ N7-Methyl-3′-O-Methylguanosine structure, e.g., CleanCap® Reagents (Trilink BioTechnologies). In some embodiments, a 5′ cap may be or comprise a dinucleotide cap analog such as G[5′]ppp[5′]G, m7G[5′]ppp[5′]G, m₃^{2,2,7}G[5′]ppp[5′]G, m₂^{7,3′-O}G[5′]ppp[5′]G (3′-ARCA), m₂^{7,2′-O}GpppG (2′-ARCA), m₂^{7,2′-O}GppSpG (D1) (β-S-ARCA(D1)), and m₂^{7,2′-O}GppSpG (D2) (β-S-ARCA (D2)) and m2^{7,3′-O}Gppp(m2′-O)ApG (CC413). In some embodiments, a 5′-capping agent is added in excess to a particular ribonucleotide or ribonucleotides (e.g., GTP, ATP, UTP, CTP, or modified version thereof) to enable incorporation of the 5′-cap as the first addition to the RNA transcript. In some embodiments, the 5′ cap used in the present invention is a m₂^{7,3′-O}Gppp(m^{2′-O})ApG 5′ cap.

In the context of the present invention, the term “5′ cap analog” refers to a molecular structure that resembles a conventional 5′ cap, but is modified to possess the ability to stabilize RNA if attached thereto, in some embodiments in vivo and/or in a cell. A cap analog is not a conventional 5′ cap.

The 5′ cap has been generally described to be involved in efficient translation of mRNA: in general, in eukaryotes, translation is initiated only at the 5′ end of a messenger RNA (mRNA) molecule, unless an internal ribosomal entry site (IRES) is present. Eukaryotic cells are capable of providing an RNA with a 5′ cap during transcription in the nucleus: newly synthesized mRNAs are usually modified with a 5′ cap structure, e.g., when the transcript reaches a length of 20 to 30 nucleotides. First, the 5′ terminal nucleotide pppN (ppp representing triphosphate; N representing any nucleoside) is converted in the cell to 5′ GpppN by a capping enzyme having RNA 5′-triphosphatase and guanylyltransferase activities. The GpppN may subsequently be methylated in the cell by a second enzyme with (guanine-7)-methyltransferase activity to form the mono-methylated m⁷GpppN cap. In some embodiments, the 5′ cap used in the present invention is a natural 5′ cap.

Presence of a cap on an RNA molecule is strongly preferred if translation of a nucleic acid sequence encoding a protein after introduction of the respective RNA into host cells or into a host organism is desired, especially if translation is desired within the first 1 hour, or within the first two hours, or within the first three hours after introduction of the RNA.

In the present invention, a natural 5′ cap dinucleotide is typically selected from the group consisting of a non-methylated cap dinucleotide (G(5′)ppp(5′)N; also termed GpppN) and a methylated cap dinucleotide ((m⁷G(5′)ppp(5′)N; also termed m⁷GpppN). m⁷GpppN (wherein N is G) is represented by the following formula:

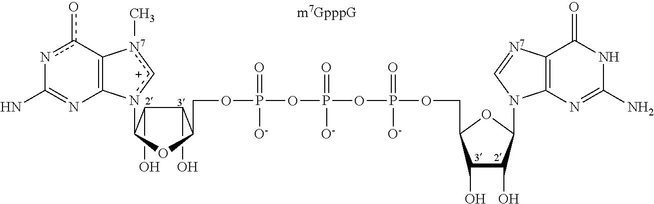

In certain embodiments of the present invention, the 5′ cap is a 5′ cap analog. 5′ cap analogs have been initially described to facilitate large scale synthesis of RNA transcripts by means of in vitro transcription.

For RNA such as mRNA, some 5′ cap analogs (synthetic caps) have been generally described to date, and they can all be used in the context of the present invention. Ideally, a 5′ cap analog is selected that is associated with higher translation efficiency and/or increased resistance to in vivo degradation and/or increased resistance to in vitro degradation.

In some embodiments, a 5′ cap analog is used that can only be incorporated into an RNA chain in one orientation. Pasquinelli et al., (1995), RNA J. 1: 957-967 demonstrated that during in vitro transcription, bacteriophage RNA polymerases use the 7-methylguanosine unit for initiation of transcription, whereby around 40-50% of the transcripts with cap possess the cap dinucleotide in a reverse orientation (i.e., the initial reaction product is Gpppm⁷GpN when m7G is used). Compared to the RNAs with a correct 5′ cap, RNAs with a reverse 5′ cap are not functional with respect to translation of a nucleic acid sequence into protein. Thus, it is desirable to incorporate the 5′ cap in the correct orientation, i.e., resulting in an RNA with a structure essentially corresponding to m⁷GpppGpN etc. It has been shown that the reverse integration of the cap-dinucleotide is inhibited by the substitution of either the 2′- or the 3′—OH group of the methylated guanosine unit (Stepinski et al., (2001), RNA J., 7: 1486-1495; Peng et al., (2002), Org. Lett., 24: 161-164). RNAs which are synthesized in presence of such “anti reverse cap analogs” or “ARCAs” are translated more efficiently than RNAs which are in vitro transcribed in presence of the conventional 5′ cap m⁷GpppG. To that end, one cap analog in which the 3′ OH group of the methylated guanosine unit is replaced by OCH₃ is described e.g. by Holtkamp et al., (2006), Blood, 108: 4009-4017 (7-methyl(3′-O-methyl)GpppG; anti-reverse cap analog (ARCA)). 7-methyl(3′-O-methyl)GpppG (sometimes also called 3′-ARCA) is a suitable cap dinucleotide according to the present invention.

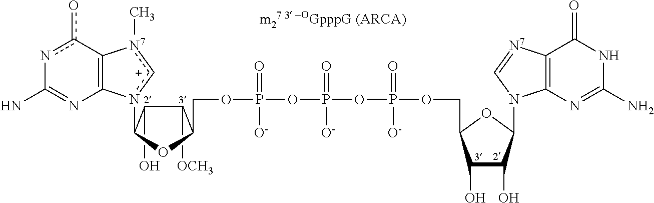

In some embodiments of the present invention, the RNA of the present invention is essentially not susceptible to decapping. This is important because, in general, the amount of protein produced from synthetic mRNAs introduced into cultured mammalian cells is limited by the natural degradation of mRNA. One in vivo pathway for mRNA degradation begins with the removal of the mRNA cap. This removal is catalyzed by a heterodimeric pyrophosphatase, which contains a regulatory subunit (Dcp1) and a catalytic subunit (Dcp2). The catalytic subunit cleaves between the α and β phosphate groups of the triphosphate bridge. In the present invention, a cap analog may be selected or present that is not susceptible, or less susceptible, to that type of cleavage. A suitable cap analog for this purpose may be selected from a cap dinucleotide according to formula (I):

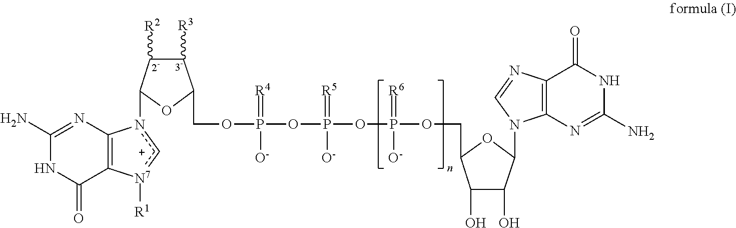

wherein R¹ is selected from the group consisting of optionally substituted alkyl, optionally substituted alkenyl, optionally substituted alkynyl, optionally substituted cycloalkyl, optionally substituted heterocyclyl, optionally substituted aryl, and optionally substituted heteroaryl,
R² and R³ are independently selected from the group consisting of H, halo, OH, and optionally substituted alkoxy, or R² and R³ together form O—X—O, wherein X is selected from the group consisting of optionally substituted CH₂, CH₂CH₂, CH₂CH₂CH₂, CH₂CH(CH₃), and
C(CH₃)₂, or R² is combined with the hydrogen atom at position 4′ of the ring to which R² is attached to form —O—CH₂— or —CH₂—O—,
R⁵ is selected from the group consisting of S, Se, and BH₃,
R⁴ and R⁶ are independently selected from the group consisting of O, S, Se, and BH₃.
n is 1, 2, or 3.

Certain embodiments for R¹, R², R³, R⁴, R⁵, R⁶ are disclosed in WO 2011/015347 A1 and may be selected accordingly in the present invention.

For example, in some embodiments of the present invention, the 5′ cap is or comprises a phosphorothioate-cap-analog. Phosphorothioate-cap-analogs are specific cap analogs in which one of the three non-bridging O atoms in the triphosphate chain is replaced with an S atom, i.e., one of R⁴, R⁵ or R⁶ in Formula (I) is S. Phosphorothioate-cap-analogs have been described by Kowalska et al., (2008), RNA, 14: 1119-1131, as a solution to the undesired decapping process, and thus to increase the stability of RNA in vivo. In particular, the substitution of an oxygen atom for a sulphur atom at the beta-phosphate group of the 5′-cap results in stabilization against Dcp2. In that embodiment, which is preferred in the present invention, R⁵ in Formula (I) is S; and R⁴ and R⁶ are O.

In some embodiments of the present invention, the RNA of the present invention comprises a phosphorothioate-cap-analog wherein the phosphorothioate modification of the RNA 5′-cap is combined with an “anti-reverse cap analog” (ARCA) modification. Respective ARCA-phosphorothioate-cap-analogs are described in WO 2008/157688 A2, and they can all be used in the RNA of the present invention. In that embodiment, at least one of R² or R³ in Formula (I) is not OH, in some embodiments one among R² and R³ is methoxy (OCH₃), and the other one among R² and R³ is in some embodiments OH. In some embodiments, an oxygen atom is substituted for a sulphur atom at the beta-phosphate group (so that R⁵ in Formula (I) is S; and R⁴ and R⁶ are O). It is believed that the phosphorothioate modification of the ARCA ensures that the α, β, and γ phosphorothioate groups are precisely positioned within the active sites of cap-binding proteins in both the translational and decapping machinery. At least some of these analogs are essentially resistant to pyrophosphatase Dep1/Dcp2. Phosphorothioate-modified ARCAs were described to have a much higher affinity for eIF4E than the corresponding ARCAs lacking a phosphorothioate group.

A respective 5′ cap analog that is particularly preferred in the present invention, i.e., m₂^{7,2′-O}GppspG, is termed β-S-ARCA (WO 2008/157688 A2; Kuhn et al., Gene Ther., (2010), 17: 961-971). Thus, in some embodiments of the present invention, the RNA of the present invention is modified with beta-S-ARCA (or β-S-ARCA). β-S-ARCA is represented by the following structure:

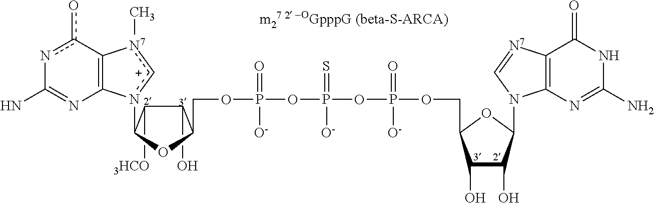

In general, the replacement of an oxygen atom for a sulphur atom at a bridging phosphate results in phosphorothioate diastereomers which are designated D1 and D2, based on their elution pattern in HPLC. Briefly, the “D1 diastereomer of 3-S-ARCA” or “β-S-ARCA(D1)” or “m₂^{7,2′-O}GppspG (D1)” is the diastereomer of β-S-ARCA which elutes first on an HPLC column compared to the D2 diastereomer of 3-S-ARCA (0-S-ARCA(D2) or m₂^{7,2′-O}GppspG (D2)) and thus exhibits a shorter retention time. Determination of the stereochemical configuration by HPLC is described in WO 2011/015347 A1.

In certain embodiments of the present invention, RNA of the present invention is modified with the β-S-ARCA(D2) diastereomer. The two diastereomers of β-S-ARCA differ in sensitivity against nucleases. It has been shown that RNA carrying the D2 diastereomer of β-S-ARCA is almost fully resistant against Dcp2 cleavage (only 6% cleavage compared to RNA which has been synthesized in presence of the unmodified ARCA 5′-cap), whereas RNA with the β-S-ARCA(D1) 5′-cap exhibits an intermediary sensitivity to Dcp2 cleavage (71% cleavage). It has further been shown that the increased stability against Dcp2 cleavage correlates with increased protein expression in mammalian cells. In particular, it has been shown that RNAs carrying the β-S-ARCA(D2) cap are more efficiently translated in mammalian cells than RNAs carrying the β-S-ARCA(D1) cap. Therefore, in some embodiments of the present invention, a 5′ cap used in the present invention is a 5′ cap analog according to Formula (I), characterized by a stereochemical configuration at the P atom comprising the substituent R⁵ in Formula (I) that corresponds to that at the P_{β} atom of the D2 diastereomer of β-S-ARCA. In that embodiment, R⁵ in Formula (I) is S; and R⁴ and R⁶ are O. Additionally, in some embodiments at least one of R² or R³ in Formula (I) is not OH, and/or one among R² and R³ is methoxy (OCH₃), and/or the other one among R² and R³ is OH; in some embodiments each of R² or R³ in Formula (I) is not OH; in some such embodiments, one among R² and R³ is methoxy (OCH₃), and the other one among R² and R³ is OH.

In certain other embodiments, a 5′ cap used in the present invention is the β-S-ARCA(D1) diastereomer. This embodiment is particularly suitable for transfer of capped RNA into immature antigen presenting cells, such as for vaccination purposes. It has been demonstrated that the β-S-ARCA(D1) diastereomer, upon transfer of respectively capped RNA into immature antigen presenting cells, is particularly suitable for increasing the stability of the RNA, increasing translation efficiency of the RNA, prolonging translation of the RNA, increasing total protein expression of the RNA, and/or increasing the immune response against an antigen or antigen peptide encoded by said RNA (Kuhn et al., (2010), Gene Ther., 17: 961-971). Therefore, in some embodiments of the present invention, RNA of the present invention is modified with a cap analog according to Formula (I), characterized by a stereochemical configuration at the P atom comprising the substituent R⁵ in Formula (I) that corresponds to that at the P_{β} atom of the D1 diastereomer of β-S-ARCA. Respective cap analogs and embodiments thereof are described in WO 2011/015347 A1 and Kuhn et al., (2010), Gene Ther., 17: 961-971. Any cap analog described in WO 2011/015347 A1, wherein the stereochemical configuration at the P atom comprising the substituent R⁵ corresponds to that at the P_{β} atom of the D1 diastereomer of β-S-ARCA, may be used in the present invention. In some embodiments, R⁵ in Formula (I) is S; and R⁴ and R⁶ are O. Additionally, in some embodiments at least one of R² or R³ in Formula (I) is not OH, and/or one among R² and R³ is methoxy (OCH₃), and/or the other one among R² and R³ is OH; in some embodiments each of R² or R³ in Formula (I) is not OH; in some such embodiments, one among R² and R³ is methoxy (OCH₃), and the other one among R² and R³ is OH; in some embodiments, the 5′ cap used in the present invention is m₂^{7,3′-O}Gppp(m₁^{2′-O})ApG (also sometimes referred to as m₂^{7,3′-O}G(5′)ppp(5′)m^{2′-O}ApG, m₂^{7,3′-O}Gppp(m₁^{2′-O})ApG, CC413 or CleanCap). In a particularly preferred embodiment, RNA of the present invention is modified with the CleanCap.

5′ caps which are useful in the present invention further include without limitation m₃^{2,2,7}G[5′]ppp[5′]G, m₂^{7,2′-O}GpppG (2′-ARCA), m⁷Gp₃ m^{2′-O}G, m⁷Gp₃ m⁷G, m2^{7,2′-O}Gp₃G, m₂^{7,2′-O}GpppSG (D1), m₂^{7,2′-O}GpppSG (D2), m₂^{7,2′-O}GppspG (D1), m₂^{7,2′-O}GppspG (D2), m₂^{7,2′-O}GpsppG (D1), m₂^{7,2′-O}GpsppG (D2). A 5′ cap useful in the present invention can also be a tetraphosphate derivative of a triphosphate 5′ cap analog, such as m⁷Gp₄G which is the derivative of m⁷Gp₃G, b⁷Gp₄G which is the derivative of m₂^{7,3′-O}Gp₃G, b⁷m^{3′-O}Gp₄G which is the derivative of b⁷Gp₃G, m₂^{2,7}Gp₄G which is the derivative of e⁷Gp₃G, m₃^{2,2,7}Gp₄G which is the derivative of m₂^{2,7}Gp₃G, b⁷m₂Gp₄G which is the derivative of m₃^{2,2,7}Gp₃G, m⁷Gp₄m⁷G which is the derivative of m⁷Gp₃2′dG. Further useful 5′ cap analogs have been described in U.S. Pat. No. 7,074,596, WO2008/016473, WO2008/157688, WO2009/149253, WO2011/015347, and WO2013/059475.

In some embodiments, a 5′ cap used in the invention is a 5′ cap structure according to Formula (I) wherein any one phosphate group is replaced by a boranophosphate group or a phosphoroselenoate group. Such 5′ caps have increased stability both in vitro and in vivo. Optionally, the respective compound has a 2′-O— or 3′-O-alkyl group (wherein alkyl, in some embodiments, is methyl); respective cap analogs are termed BH₃-ARCAs or Se-ARCAs. Compounds that are particularly suitable for capping of mRNA include the β-BH₃-ARCAs and β-Se-ARCAs, as described in WO 2009/149253. For these compounds, a stereochemical configuration at the P atom comprising the substituent R⁵ in Formula (I) that corresponds to that at the P_{β} atom of the D1 diastereomer of beta-S-ARCA is preferred.

### Exemplary In Vitro Transcription Reactions

One of ordinary skill in the art will understand materials and reagents for a typical in vitro transcription. In some embodiments, an individual reaction component or components are thawed prior to their addition to an in vitro transcription reaction mixture. For example, an in vitro transcription reaction mixture typically includes a DNA template (e.g., as described herein), ribonucleotides (e.g., as described herein), a RNA polymerase (e.g., DNA dependent RNA polymerases), and an appropriate reaction buffer for a selected RNA polymerase. In some embodiments, an in vitro transcription reaction mixture may further comprise an RNase inhibitor. In some embodiments, an in vitro transcription reaction mixture may further comprise a pyrophosphatase (e.g., an inorganic pyrophosphatase). In some embodiments, an in vitro transcription reaction mixture may further comprise one or more salts (e.g., monovalent salts and/or divalent salts such as Mg2+), a reducing agent (e.g., dithithreitol, 2-mercaptoethanol, etc.), spermidine, or combinations thereof. In some embodiments, certain reaction components are added in a specific order (e.g., pyrophosphatase and polymerase added last). In some embodiments, agitation rate is increased following the addition of specific reaction components (e.g., pyrophosphatase, polymerase).

Various RNA polymerases that are suitable for in vitro transcription are known in the art, including, e.g., but not limited to DNA dependent RNA polymerases (e.g., a T7 RNA polymerase, a T3 RNA polymerase, a SP6 RNA polymerase, a N4 virion RNA polymerase, or a variant or functional domain thereof). A skilled artisan will understand that an RNA polymerase utilized herein may be a recombinant RNA polymerase, and/or a purified RNA polymerase, i.e., not as part of a cell extract, which contains other components in addition to the RNA polymerases. In some embodiments, an RNA polymerase that is useful for commercial-scale in vitro transcription is a T7 RNA polymerase. In some embodiments, an inorganic pyrphosphatase may be added to improve the yield of in vitro transcription reaction (e.g., in some embodiments catalyzed by T7 RNA polymerase).

In some embodiments, a buffer used in a transcription reaction (transcription buffer) is optimized for a selected RNA polymerase. Transcription buffer is typically optimized for a selected RNA polymerase. For example, in some embodiments, a transcription buffer may comprise Tris-HCl, HEPES, or other appropriate buffer. In some embodiments, a transcription buffer can comprise 20-60 mM HEPES, 20-60 mM divalent salt (e.g., magnesium salts, such as magnesium chloride, magnesium acetate, Li⁺, Na⁺, K⁺, NH⁴⁺, tris(hydroxymethyl)aminomethane cation, Mg²⁺, Ba²⁺ or Mn²⁺, etc.), 5-15 mM reducing agent (e.g., dithiothreitol, 2-mercaptoethanol, etc.) and 0.5-3 mM spermidine.

In some embodiments, a transcription reaction is conducted at a pH of about 6, 6.5, 7, 7.5, 8, or 9. In some embodiments, a transcription buffer has a pH of 7-9 (e.g., about 7.1, 7.2, 7.3, 7.4, 7.5, 7.6 7.7, 7.8, 7.9, 8.0, 8.1, 8.2, 8.3, 8.4, 8.5, 8.6, 8.7, 8.8, 8.9, 9.0). In some embodiments, a transcription buffer has a pH of 6-9. In some embodiments, the pH value is about 6.0, 6.1, 6.2, 6.3, 6.4, 6.5, 6.6, 6.7, 6.8, 6.9, 7.0, 7.1, 7.2, 7.3, 7.4, 7.5, 7.6 7.7, 7.8, 7.9, 8.0, 8.1, 8.2, 8.3, 8.4, 8.5, 8.6, 8.7, 8.8, 8.9, 9.0. In some embodiments, a transcription buffer has a pH value of about 6-8.5. In some embodiments the buffer has a pH value from about 6 to 8.5, from about 6.5 to 8.0, from about 7.0 to 7.5, in some embodiments about 7.5. In some embodiments, a suitable pH value for a transcription reaction may be approximately 7.5-8.5. In some embodiments, the pH value is about 6.0, 6.1, 6.2, 6.3, 6.4, 6.5, 6.6, 6.7, 6.8, 6.9, 7.0, 7.1, 7.2, 7.3, 7.4, 7.5, 7.6 7.7, 7.8, 7.9, 8.0, 8.1, 8.2, 8.3, 8.4, 8.5, 8.6, 8.7, 8.8, 8.9, 9.0. In some embodiments, the pH value is about 6-8.5. In some embodiments the pH value is from 6 to 8.5, from 6.5 to 8.0, from 7.0 to 7.5; in some embodiments, the pH value is 7.5.

In some embodiments, the pH value of the reaction mix is kept essentially constant over the course of the transcription reaction, e.g., by using a suitable buffer. Buffers suitable for adjusting pH value are known in the art and described herein, and include without limitation NaOH buffer, KOH buffer or HCl buffer.

In some embodiments, the pH value of the reaction mix is kept substantially constant during the course of the transcription reaction, e.g., by supplementing buffer with a pH value similar or equal to the pH value of the starting reaction mix and/or by supplementing buffer with a pH value different to the pH value of the starting reaction mix, if required.

In some embodiments, a buffer is selected from the group consisting of 80 mM HEPES/KOH, pH 7.5 and 40 mM Tris/HCl, pH 7.5.

### Exemplary In Vitro Transcription Reaction Conditions

In some embodiments, an in vitro transcription reaction is conducted, e.g., in a bioreactor described herein (selected for a certain in vitro transcription reaction volume, e.g., as described herein) for a period of time. In some embodiments, the period of time is at least 20 minutes, including, e.g., at least 25 minutes, at least 30 minutes, at least 40 minutes, at least 55 minutes, at least 60 minutes, at least 75 minutes, at least 90 minutes, at least 105 minutes, at least 120 minutes, at least 135 minutes, at least 150 minutes, at least 165 minutes, or at least 180 minutes. In some embodiments, the period of time is 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 minutes. In some embodiments, the period of time is about 1.5-3 hours. In some embodiments, the period of time is about 25-35 minutes.

In some embodiments, an in vitro transcription reaction is conducted, e.g., in a bioreactor described herein for a period of time (e.g., as described herein) at a temperature at which a selected RNA polymerase is functionally active. While typical phage RNA polymerases (e.g., T7 polymerases) that carry out in vitro transcription reactions are usually not active at elevated temperatures (e.g., above 45° C.), thermostable RNA polymerases (e.g., thermostable variants of T7 RNA polymerases such as ones as described in U.S. Ser. No. 10/519,431, the contents of which are incorporated by reference for purposes described herein) can show increased stability at elevated temperatures. In some embodiments, in vitro transcription is performed at a temperature of approximately 25° C. or higher, including, e.g., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., or 45° C. In some embodiments, in vitro transcription is performed at a temperature of approximately 45° C. or higher, including, e.g., 46° C., 47° C., 48° C., 49° C., 50° C., 51° C., 52° C., 53° C., 54° C., 55° C. or higher.

In some embodiments, an in vitro transcription is conducted e.g., in a bioreactor described herein at a pH of about 6, 6.5, 7, 7.5, 8, or 9. In some embodiments, a suitable pH for an in vitro transcription may be approximately 7.5-8.5.

In some embodiments, in vitro transcription reactions performed in accordance with the present disclosure (e.g., in a bioreactor as described herein) may be performed as continuous feed reactions; in some embodiments, they may be performed as batch-fed reactions. In some embodiments, one or more nucleotides may be added to an in vitro transcription reaction in a step-wise manner (e.g. at least 5, 6, 7, 8, 9, 10, 11, 12, 13, 14, 15, or more bolus feeds). In some embodiments, an agitation rate is selected such that a particular blend time to enable rapid mixing of bolus additions to ensure optimal availability of modified nucleotide solution and one or more other nucleotide solutions during RNA synthesis is achieved.

Limitation of reaction components: In certain embodiments, a limiting component is present in a starting concentration that is lower than the starting concentration of a non-limiting component. In some embodiments, a limiting component is a nucleotide such as ATP, GTP, CTP, UTP or functional analogs thereof. In some embodiments, a non-limiting component is a nucleotide such as ATP, GTP, CTP, UTP or functional analogs thereof. A limiting or non-limiting component can also be a 5′ cap or 5′ cap analog. In some embodiments, the ratio of a limiting component to a non-limiting component, such as the ratio of UTP or a functional analog thereof to ATP and/or CTP, or a functional analog thereof, or the ratio of GTP or a functional analog thereof to ATP and/or CTP, or a functional analog thereof, is between about 1:1 and about 1:100, such as between 1:1.1 and about 1:80, between 1:1.2 and about 1:60, between 1:1.3 and about 1:40, in some embodiments between 1:1.4 and about 1:30, in some embodiments between 1:1.5 and about 1:20, in some embodiments between 1:1.5 and about 1:15, between 1:1.6 and about 1:10, between 1:1.7 and about 1:9, between 1:1.8 and about 1:8, between 1:1.9 and about 1:7, between 1:2 and about 1:6. In some embodiments, the starting concentration of one or more limiting components, such as UTP or a functional analog thereof or GTP or a functional analog thereof, is 1/2, 1/3, 1/4, 1/5, 1/6, 1/7, 1/8, 1/9, 1/10, 1/11, 1/12, 1/13, 1/14, 1/15, 1/16, 1/17, 1/18, 1/19, 1/20, 1/21, 1/22, 1/23, 1/24, 1/25, 1/26, 1/27, 1/28, 1/29, 1/30, 1/31, 1/32, 1/33, 1/34, 1/35, 1/36, 1/37, 1/38, 1/39, 1/40, 1/50, 1/60, 1/70, 1/80, 1/90 or 1/100 when compared to the starting concentration of one or more non-limiting components, such as ATP and/or CTP, or a functional analog thereof.

UTP limitation and/or supplementation: In some embodiments, an in vitro transcription reaction comprises UTP or a functional thereof at a limiting concentration in combination with at least one or all of ATP or a functional analog thereof, CTP or a functional analog thereof, and optionally GTP or a functional analog thereof. In some embodiments, a functional analog of UTP is or comprises N1-methylpseudouridine-5′ triphosphate (m1ΨTP). In some embodiments, UTP or a functional analog thereof is present in an in vitro transcription reaction at a starting concentration that limits the rate of transcription. In some embodiments, UTP or a functional analog thereof is present in an in vitro transcription reaction at a starting concentration that is lower than the starting concentration of at least one or all of ATP or a functional analog thereof, CTP or a functional analog thereof, and optionally GTP or a functional analog thereof. In some embodiments, the starting concentration of UTP or a functional analog thereof is at least 30% lower (including, e.g., at least 40%, at least 50%, at least 60%, at least 70%, at least 80%, at least 90% lower) than the starting concentration of at least one or all of ATP or a functional analog thereof, CTP or a functional analog thereof, and optionally GTP or a functional analog thereof. In some embodiments, the ratio of the starting concentration of UTP or a functional analog thereof to the starting concentration of at least one or all of ATP or a functional analog thereof, CTP or a functional analog thereof, and optionally GTP or a functional analog thereof is about 1:1.3 or lower, including, e.g., 1:1.4; 1:1.5; 1:2, 1:2.5; 1:3; 1:3.5; 1:4; 1:4.5; 1:5; 1:6; 1:7; 1:8, 1:9; 1:10; 1:11; 1:12; 1:13; 1:14; 1:15; 1:16; 1:17; 1:18; 1:19; 1:20, or lower. In some embodiments, the ratio of the starting concentration of UTP or a functional analog thereof to the starting concentration of at least one or all of ATP or a functional analog thereof, CTP or a functional analog thereof, and optionally GTP or a functional analog thereof is about 1:1.3 to about 1:20, or about 1:1.5 to about 1:15, or about 1:5 to about 1:15, or about 1:8 to about 1:12. In some such embodiments, the starting concentration of ATP or a functional analog thereof, CTP or a functional analog thereof, and optionally GTP or a functional analog thereof may be the same.

In some embodiments, an in vitro transcription reaction is supplemented at least once with UTP or a functional analog thereof over the course of the reaction. In some embodiments, an in vitro transcription reaction is supplemented multiple times (e.g., at least 2 or more, including, e.g., 2, 3, 4, 5, 6, 7, 8, 9, 10, 11, 12, 13, 14, 15 or more) with UTP or a functional analog thereof over the course of the transcription reaction. In some embodiments, supplementation of UTP or a functional analog thereof is performed when its concentration in the reaction mixture is near depletion. In some embodiments, supplementation of UTP or a functional analog thereof is performed when its concentration in the reaction mixture is less than 100 uM, 90 uM, 80 uM, 70 uM, 60 uM, 50 uM, 40 uM, 30 uM, 20 uM, 10 uM, 5 uM, 3 uM, 2, uM, 1 uM, 500 nM, 250 nM, 200 nM, 100 nM, 50 nM, 25 nM, or lower. In some embodiments, supplementing UTP or a functional analog thereof may comprise supplementing UTP or a functional analog thereof and supplementing GTP or a functional analog thereof, e.g., as a composition, optionally comprising further reaction components as described herein. In other embodiments, supplementing UTP does not refer to supplementing other reaction components. Likewise, in some embodiments, supplementing GTP does not refer to supplementing other reaction components.

In some embodiments, UTP (or a functional analog thereof) supplementation may be performed continuously during the course of the transcription reaction. For example, in some embodiments, UTP (or a functional analog thereof) supplementation may be performed in a continuous manner at a rate that is comparable to (e.g., within 10% or lower) of its consumption rate. In some embodiments, UTP (or a functional analog thereof) supplementation may be performed at a rate such that after such supplementation, UTP or a functional analog thereof is present in the reaction at a concentration lower than that of one or more, and in some embodiments, all of ATP or a functional analog thereof, GTP or a functional analog thereof, and/or CTP or a functional analog thereof.

In some embodiments, UTP (or a functional analog thereof) supplementation may be performed periodically during the course of the transcription reaction. In some embodiments, UTP (or a functional analog thereof) supplementation may be performed in a periodic manner such that after each addition, UTP or functional analog thereof is present in the reaction at a concentration lower than that of one or more, and in some embodiments, all of ATP or functional analog thereof, GTP or functional analog thereof, and/or CTP or functional analog thereof. In some embodiments, such periodic supplementation may be performed as one or more bolus or batch addition(s), including, e.g., 1, 2, 3, 4, 5, 6, 7, 8, 9, 10, 11, 12, 13, 14, 15 or more bolus or batch addition(s). In some embodiments, such periodic supplementation may be performed by a fed-batch process. In some embodiments, supplementing may comprise supplementing a composition comprising UTP or a functional analog thereof and comprising further components such as buffer, polymerase, CTP or a functional analog thereof, GTP or a functional analog thereof, ATP or a functional analog thereof, or other components that may be present in a transcription reaction mix as described herein. In some embodiments, supplementing UTP or a functional analog thereof does not comprise supplementing CTP or ATP, or functional analogs thereof.

In some embodiments, the concentration of UTP or a functional analog thereof added during supplementation is same as the starting concentration of UTP or a functional analog thereof. In some embodiments, the concentration of UTP or a functional analog thereof added during supplementation is lower than the starting concentration of UTP or a functional analog thereof, e.g., at least 10% lower (including, e.g., at least 20%, at least 30%, at least 40%, at least 50%, at least 60%, at least 70%, at least 80%, at least 90% lower) than the starting concentration of UTP or a functional analog thereof.

In some embodiments, UTP (or a functional analog thereof) supplementation is performed at a concentration and/or at a rate or manner such that the ratio of the concentration of UTP or a functional analog thereof to the concentration of at least one or all of ATP or a functional analog thereof, CTP or a functional analog thereof, and optionally GTP or a functional analog thereof (during the course of the reaction) is maintained substantially the same (e.g., within 10% or less) as the initial ratio of the concentration of UTP or a functional analog thereof to the starting concentration of at least one or all of ATP or a functional analog thereof, CTP or a functional analog thereof, and optionally GTP or a functional analog thereof (at the beginning of the reaction).

In some embodiments, UTP or a functional analog thereof is supplemented until the end of the transcription reaction.

In some embodiments, UTP or a functional analog thereof is present in an initial transcription reaction at a starting concentration of 0.1 to 2 mM or 0.1 to 1.5 mM, or 0.1 to 1 mM, or 0.5 to 2 mM, or 1 to 2 mM. In some embodiments, UTP or a functional analog thereof is maintained during the course of an in vitro transcription reaction at a concentration of 0.1 to 2 mM or 0.1 to 1.5 mM, or 0.1 to 1 mM, or 0.5 to 2 mM, or 1 to 2 mM.

Optional additional non-UTP limitation and/or supplementation: In some embodiments, at least one of non-UTP (or functional analog thereof) is provided at a limiting concentration (in addition to limited UTP or a functional analog thereof) at the initial in vitro transcription reaction (e.g., the beginning of the in vitro transcription reaction). For example, in some embodiments, at least one of ATP or a functional analog thereof, CTP or a functional analog thereof, or GTP or a functional analog thereof is provided at a limiting concentration (in addition to limited UTP or a functional analog thereof) at the initial in vitro transcription reaction (e.g., the beginning of the in vitro transcription reaction). In some embodiments, GTP or a functional analog thereof is provided at a limiting concentration (in addition to limited UTP or a functional analog thereof) at the initial in vitro transcription (e.g., the beginning of the in vitro transcription reaction).

In certain embodiments of the invention, methods involve supplementing a transcription and capping reaction with GTP or a functional analog thereof because it competes with a cap analog in certain reactions, such as when T7, SP6 or T3 polymerase is used to catalyze the reaction. It will be understood, however, that the invention is not limited to GTP or a functional analog thereof. Instead, the invention can be implemented with respect to any reaction involving a nucleotide that competes with a cap analog or a nonextending mono- or di-nucleotide that can be incorporated at the 5′ end of the transcript. Thus, it is specifically contemplated that any embodiment involving GTP or a functional analog thereof as the competing nucleotide can be implemented with respect to a different nucleotide or nucleotide analog. The method does not depend on whether GTP and/or a functional analog thereof are used, so long as the analog is incorporated at a rate similar to GTP by the polymerase into the elongated transcript. The term “functional analog of GTP” as used herein, refers to extending nucleotides, and thus, excludes any cap analogs, as defined below.

In some embodiments, a starting concentration of GTP or a functional analog thereof limits the rate of transcription. Using a starting concentration of GTP or a functional analog thereof that limits the rate of transcription of a transcription and/or capping reaction and supplementing the reaction with GTP or a functional analog thereof is preferred because GTP competes with a 5′ cap or 5′ cap analog in certain reactions such as reactions using T7, SP6 or T3 polymerase. It will be understood, however, that the invention is not limited to GTP or a GTP analog. In certain embodiments, UTP or a functional analog thereof is present in a starting amount that is limiting to the reaction and a method according to the invention comprises supplementing the reaction mix with UTP or a functional analog thereof. In certain embodiments, UTP or a functional analog thereof and GTP or a functional analog thereof are present in a starting amount that is limiting to the reaction and a method according to the invention comprises supplementing the reaction mix with UTP or a functional analog thereof and GTP or a functional analog thereof.

In some embodiments, GTP or a functional analog thereof is present in an in vitro transcription reaction at a starting concentration that limits the rate of transcription. In some embodiments, GTP or a functional analog thereof is present in an in vitro transcription reaction at a starting concentration that is lower than the starting concentration of at least one or all of ATP or a functional analog thereof and/or CTP or a functional analog thereof. In some embodiments, the starting concentration of GTP or a functional analog thereof is at least 30% lower (including, e.g., at least 40%, at least 50%, at least 60%, at least 70%, at least 80%, at least 90% lower) than the starting concentration of at least one or all of ATP or a functional analog thereof and/or CTP or a functional analog thereof. In some embodiments, the ratio of the starting concentration of GTP or a functional analog thereof to the starting concentration of at least one or all of ATP or a functional analog thereof and/or CTP or a functional analog thereof is about 1:1.3 or lower, including, e.g., 1:1.4; 1:1.5; 1:2, 1:2.5; 1:3; 1:3.5; 1:4; 1:4.5; 1:5; 1:6; 1:7; 1:8, 1:9; 1:10; 1:11; 1:12; 1:13; 1:14; 1:15; 1:16; 1:17; 1:18; 1:19; 1:20, or lower. In some embodiments, the ratio of the starting concentration of GTP or a functional analog thereof to the starting concentration of at least one or all of ATP or a functional analog thereof and/or CTP or a functional analog thereof is about 1:1.3 to about 1:20, or about 1:1.5 to about 1:15, or about 1:5 to about 1:15, or about 1:8 to about 1:12. In some such embodiments, the starting concentration of ATP or a functional analog thereof and/or CTP or a functional analog thereof.

In some embodiments, an in vitro transcription reaction is supplemented at least once with GTP or a functional analog thereof over the course of the reaction. In some embodiments, an in vitro transcription reaction is supplemented multiple times (e.g., at least 2 or more, including, e.g., 2, 3, 4, 5, 6, 7, 8, 9, 10, 11, 12, 13, 14, 15 or more) with GTP or a functional analog thereof over the course of the transcription reaction. In some embodiments, supplementation of GTP or a functional analog thereof is performed when its concentration in the reaction mixture is near depletion. In some embodiments, supplementation of GTP or a functional analog thereof is performed when its concentration in the reaction mixture is less than 100 uM, 90 uM, 80 uM, 70 uM, 60 uM, 50 uM, 40 uM, 30 uM, 20 uM, 10 uM, 5 uM, 3 uM, 2, uM, 1 uM, 500 nM, 250 nM, 200 nM, 100 nM, 50 nM, 25 nM, or lower.

In some embodiments, GTP (or a functional analog thereof) supplementation may be performed continuously during the course of the transcription reaction. For example, in some embodiments, GTP (or a functional analog thereof) supplementation may be performed in a continuous manner at a rate that is comparable to (e.g., within 10% or lower) of its consumption rate. In some embodiments, GTP (or a functional analog thereof) supplementation may be performed at a rate such that after such supplementation, GTP or functional analog thereof is present in the reaction at a concentration lower than that of ATP or functional analog thereof and/or CTP or functional analog thereof.

In some embodiments, GTP (or a functional analog thereof) supplementation may be performed periodically during the course of the transcription reaction. In some embodiments, GTP (or a functional analog thereof) supplementation may be performed in a periodic manner such that after each addition, GTP or functional analog thereof is present in the reaction at a concentration lower than that of one or more, and in some embodiments, all of ATP or functional analog thereof, and/or CTP or functional analog thereof. In some embodiments, such periodic supplementation may be performed as one or more bolus or batch addition(s), including, e.g., 1, 2, 3, 4, 5, 6, 7, 8, 9, 10, 11, 12, 13, 14, 15 or more bolus or batch addition(s). In some embodiments, such periodic supplementation may be performed by a fed-batch process. In some embodiments, supplementing may comprise supplementing a composition comprising GTP or a functional analog thereof and comprising further components such as buffer, polymerase, CTP or a functional analog thereof, UTP or a functional analog thereof, ATP or a functional analog thereof, or other components that may be present in a transcription reaction mix as described herein. In some embodiments, supplementing GTP or a functional analog thereof does not comprise supplementing ATP or CTP, or functional analogs thereof.

In some embodiments, the concentration of GTP or a functional analog thereof added during supplementation is same as the starting concentration of GTP or a functional analog thereof. In some embodiments, the concentration of GTP or a functional analog thereof added during supplementation is lower than the starting concentration of GTP or a functional analog thereof, e.g., at least 10% lower (including, e.g., at least 20%, at least 30%, at least 40%, at least 50%, at least 60%, at least 70%, at least 80%, at least 90% lower) than the starting concentration of GTP or a functional analog thereof.

In some embodiments, GTP (or a functional analog thereof) supplementation is performed at a concentration and/or at a rate or manner such that the ratio of the concentration of GTP or a functional analog thereof to the concentration of ATP or a functional analog thereof, and/or CTP or a functional analog thereof (during the course of the reaction) is maintained substantially the same (e.g., within 10% or less) as the initial ratio of the concentration of GTP or a functional analog thereof to the starting concentration of ATP or a functional analog thereof and/or CTP or a functional analog thereof (at the beginning of the reaction).

In some embodiments, GTP or a functional analog thereof is supplemented until the end of the transcription reaction.

In some embodiments, GTP or a functional analog thereof is present in an initial transcription reaction at a starting concentration of 0.1 to 2 mM or 0.1 to 1.5 mM, or 0.1 to 1 mM, or 0.5 to 2 mM, or 1 to 2 mM. In some embodiments, GTP or a functional analog thereof is maintained during the course of an in vitro transcription reaction at a concentration of 0.1 to 2 mM or 0.1 to 1.5 mM, or 0.1 to 1 mM, or 0.5 to 2 mM, or 1 to 2 mM.

In some embodiments, non-UTP supplementation does not include supplementation of CTP or functional analog thereof or ATP or functional analog thereof.

In some embodiments where non-UTP supplementation is performed, such non-UTP supplementation can be performed concurrently with UTP supplementation over the course of the reaction. In some embodiments, non-UTP or functional analog thereof and UTP or functional analog thereof can be added to a reaction mixture as a single composition. In some embodiments, non-UTP or functional analog thereof and UTP or functional analog thereof can be added to a reaction mixture as separate compositions, for example, each at the same or different concentrations and/or each introduced at the same or different flow rates to a reaction mixture). In some embodiments, such non-UTP supplementation and UTP supplementation can be performed by different methods, e.g., one is performed continuously (e.g., as described herein) while another is performed periodically (e.g., as described herein).

In some embodiments, supplementing a nucleotide such as UTP and/or GTP, or a functional analog thereof, includes supplementing more than one type of nucleotide, e.g., supplementing more than one functional analog of UTP and/or GTP. For example, in some embodiments, supplementing the reaction mix during the transcription reaction comprises supplementing UTP and pseudo-UTP. In some embodiments, supplementing the reaction mix during the transcription reaction comprises supplementing UTP, pseudo-UTP and GTP. In some embodiments, supplementing the reaction mix during the transcription reaction comprises supplementing pseudo-UTP and/or 1-methylpseudo-UTP and GTP. In some embodiments, supplementing more than type of nucleotide results in supplementing amounts of UTP and/or GTP or functional analogs thereof that result in excess of UTP and/or GTP or functional analogs thereof in the reaction mix. In some embodiments, supplementing more than one functional analog of UTP and/or GTP does not result in supplementing amounts of UTP and/or GTP or functional analogs thereof that would result in excess of UTP and/or GTP or functional analogs thereof in the reaction mix.

In some embodiments, a method for increasing the yield of capped RNA transcript and/or for decreasing dsRNA comprise: incubating components for a transcription and capping reaction under conditions to promote polymerization of the transcript, wherein the concentration of a 5′ cap analog is maintained in the reaction at a ratio of between about 1:1 and about 50:1 relative to the concentration of a competing nucleotide component by multiple administration of the competing nucleotide component. In specific embodiments, the competing nucleotide is GTP or a functional analog thereof. In reactions involving T7, T3, or SP6 RNA polymerase, the competing nucleotide is typically GTP, or a functional analog thereof. It is specifically contemplated that any embodiment involving the use of GTP or a functional analog thereof may be substituted with another nucleotide triphosphate or functional analog thereof when using an RNA polymerase that employs that particular nucleotide at the +1 position. The present invention also relates to methods for increasing the yield of capped transcripts and/or for decreasing dsRNA in an in vitro transcription and capping reaction comprising: incubating reaction components under conditions that enable transcription, wherein the concentration of GTP or a functional analog thereof in the reaction is maintained at a concentration between about 0.2 mM and about 2.0 mM and the concentration of other nucleotides is at least about 0.2 mM for at least 30 minutes during the reaction.

Moreover, the present invention relates to methods of producing RNA with a nonextending nucleotide at the 5′ end comprising introducing a nucleotide that competes with the non-extending nucleotide by a fed-batch process to a transcription reaction comprising RNA polymerase and the non-extending nucleotide. In particular embodiments, the non-extending nucleotide is not a functional cap analog. It is specifically contemplated that any embodiment discussed with respect to GTP or a GTP analog may be implemented with respect to another nucleotide so long as that nucleotide competes with a non-extending nucleotide at the 5′ end, and vice versa. Furthermore, it will also be understood that any embodiment discussed with respect to a 5′ cap or 5′ cap analog can be implemented with respect to a nonextending nucleotide capable of being added only to the 5′ end of the transcript, and vice versa.

In certain embodiments, the reaction can be supplemented with a 5′ cap or a 5′ cap analog during the course of the transcription and/or capping reaction. In certain embodiments, the reaction is not supplemented with a 5′ cap or a 5′ cap analog during the course of the transcription and/or capping reaction.

In some embodiments, one of the components supplemented to the reaction, e.g., by a fed-batch process is a nucleotide. In some cases, more than one nucleotide is introduced by the fed-batch process. For example, both UTP and GTP nucleotides or functional analogs thereof may be supplemented by a fed-batch process, or UTP and a functional analog thereof may be supplemented by a fed-batch process, and/or GTP and a functional analog thereof may be supplemented by a fed-batch process. In further embodiments, all of the nucleotides are supplemented by a fed-batch process. One or more of the nucleotides in the reaction may be a modified nucleotide such as a functional analog of a nucleoside triphosphate described herein. Non-cap nucleotides may be modified but still be functional in that they may be incorporated at the 3′ end onto a polymerized transcript; that is, these non-cap modified nucleotides are extendable because they have a 5′ triphosphate.

In some embodiments, a programmable pump may be used for supplementation. In some embodiments, a programmable syringe pump may be used, for example, to automatically perform step-wise addition of one or more reaction components. Alternatively or additionally, in some embodiments, a monitor (e.g., a sensor) may be utilized to detect level(s) of one or more components; in some such embodiments, a monitor may communicate automatically with a pump, for example so that additional feeds may be released upon detection of a reduced amount of such component(s).

In some embodiments, following RNA transcription, a DNA template can be removed or separated from an in vitro transcription RNA composition, for example using methods known in the art, e.g., DNA hydrolysis.

In some embodiments, an RNase inhibitor may be added during DNA removal or digestion to protect RNA from potential degradation. In some embodiments, a chelating agent may be added to a DNase-treated transcription mixtures to complex with divalent ions that may be added during in vitro transcription reaction. An exemplary chelating agent may be or comprise ethylenediaminetetraacetic acid (EDTA). In some embodiments, upon addition of chelating agent, the temperature may be shifted at least 1° C. (including e.g., at least 2° C., 3° C., 4° C., 5° C., 6° C., 7° C., 8° C., 9° C., 10° C. or more).

### Bioreactor

In some embodiments, a transcription reaction is conducted in, (i.e., using) a, bioreactor described herein.

As noted herein, in some embodiments, a transcription reaction is conducted at a pH of about 6, 6.5, 7, 7.5, 8, or 9. In some embodiments, a suitable pH value for a transcription reaction may be approximately 7.5-8.5. In some embodiments, the pH value is about 6.0, 6.1, 6.2, 6.3, 6.4, 6.5, 6.6, 6.7, 6.8, 6.9, 7.0, 7.1, 7.2, 7.3, 7.4, 7.5, 7.6 7.7, 7.8, 7.9, 8.0, 8.1, 8.2, 8.3, 8.4, 8.5, 8.6, 8.7, 8.8, 8.9, 9.0. In some embodiments, the pH value is about 6-8.5. In some embodiments the pH value is from 6 to 8.5, from 6.5 to 8.0, from 7.0 to 7.5; in some embodiments, the pH value is 7.5. In some embodiments, the pH value of the reaction mix is kept essentially constant over the course of the transcription reaction, e.g., by using a suitable buffer. Buffers suitable for adjusting pH value are known in the art and described herein, and include without limitation NaOH buffer, KOH buffer or HCl buffer. In some embodiments the buffer has a pH value as described herein, e.g., from 6 to 8.5, from 6.5 to 8.0, from 7.0 to 7.5, or 7.5. In some embodiments, a buffer is selected from the group consisting of 80 mM HEPES/KOH, pH 7.5 and 40 mM Tris/HCl, pH 7.5. In some embodiments, keeping the pH value constant and/or monitoring the pH value over the course of a transcription reaction is done in, i.e., using a bioreactor.

In some embodiments, the progress of the transcription reaction is monitored in real time. In some embodiments, monitoring the progress of the transcription reaction is accomplished using a bioreactor such as a bioreactor comprising a sensor, e.g., an UV flow cell for UV 260/280 nm measurement.

The term “bioreactor” or “transcription reactor” as used herein refers to a vessel such as a chamber or test tube or column, wherein a transcription reaction is carried out under specific conditions such as described herein. Bioreactors for transcription are known in the art (see WO 1995/08626 and EP 3 155 129). A bioreactor typically is configured such that reaction components are delivered by a feed line to the reactor core and RNA products are removed by passing through an ultrafiltration membrane (see EP 3 155 129 and van de Merbel, (1999), J. Chromatogr. A 856(1-2): 55-82) to the exit stream. A bioreactor useful in methods of the present invention may comprise a reaction module for carrying out transcription reactions, a capture module for temporarily capturing the transcribed RNA molecules, and a control module for controlling the infeed of components of the reaction mix into the reaction module, wherein the reaction module may comprise a filtration membrane for separating nucleotides from the reaction mix, and the control of the infeed of components of the reaction mix by the control module may be based on a measured concentration of separated nucleotides. The bioreactor may be thermally regulated to maintain accurately a specific temperature such as the temperature of the transcription reaction as described herein, e.g., usually between 4° C. and 40° C. The bioreactor may comprise an inflow feet and an exit port. The bioreactor may allow for stirring the reaction mix during the transcription reaction, e.g., at variable rates of stirring. Stirring may be continuous or discontinuous such as in intervals.

A bioreactor for use according to the invention can be of any size so long as it is useful for transcription. For example, in some embodiments, a bioreactor can be at least 0.2 liter or more, such as 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5, 6, 7, 8, 9, 10, 15, 20, 25, 30, 35, 40, 45, 50 liters or more, or any volume in between. The internal conditions of the bioreactor, including, but not limited to pH and temperature, are typically controlled during a transcription reaction as described herein. The bioreactor can be composed of any material that is suitable for in vitro transcription under the conditions as described herein, including glass, plastic or metal.

In some embodiments, a bioreactor may be equipped with a pump for supplementing the reaction mix. In some embodiments, a programmable pump may be used for supplementation. In some embodiments, a programmable syringe pump may be used, for example, to automatically perform step-wise addition of one or more reaction components. Alternatively or additionally, in some embodiments, a monitor (e.g., a sensor) may be utilized to detect level(s) of one or more components; in some such embodiments, a monitor may communicate automatically with a pump, for example so that additional feeds may be released upon detection of a reduced amount of such component(s).

### Uses

In many embodiments, RNA product produced by methods described herein has a reduced amount of dsRNA contaminant, as compared to RNA product produced by a process in which UTP is limiting during in vitro transcription process. In some embodiments, such RNA product has a low level of dsRNA contaminant that does not require a purification process to remove dsRNA contaminant. In some embodiments, such RNA (e.g., mRNA) is therapeutic. In some embodiments, such RNA (e.g., mRNA) is a pharmaceutical-grade product.

In some embodiments, one or more RNAs (e.g., single-stranded RNAs) can be formulated with lipid nanoparticles or complexed with liposomes to produce pharmaceutical-grade compositions or preparations comprising RNA-LNPs or RNA-lipoplexes. In some embodiments, lipid nanoparticles are lipid nanoparticles comprising one or more cationic or ionizable lipids, e.g., as known in the art. In some embodiments, lipid nanoparticles may comprise at least one cationic or ionizable lipid, at least one polymer-conjugated lipid, and at least one helper lipid (e.g., at least one neutral lipid such as, e.g., a phospholipid and/or sterol).

In some embodiments, such RNA product described herein can be administered to subject in need thereof (e.g., who would benefit from expression of encoded polypeptide—for example as replacement or to stimulate or enhance immune response), typically in some embodiments via incorporation in LNP.

Treatment of a condition and Vaccination: In many embodiments, provided RNA products (e.g., preparations manufactured through use of a process as described herein) are utilized for treatment of a condition and/or for vaccination.

The term “immunization” or “vaccination” generally refers to a process of treating a subject for therapeutic or prophylactic reasons. A treatment, particularly a prophylactic treatment, is or comprises preferably a treatment aiming to induce or enhance an immune response of a subject, e.g. against one or more antigens. If, according to the present invention, it is desired to induce or enhance an immune response by using RNA as described herein, the immune response may be triggered or enhanced by the RNA. In some embodiments, the invention provides a prophylactic treatment which is or comprises preferably the vaccination of a subject. An embodiment of the present invention wherein the RNA of the invention encodes, as a protein of interest, a pharmaceutically active peptide or protein which is an immunologically active compound or an antigen is particularly useful for vaccination. When aimed at treating a condition, RNA of the invention preferably encodes a peptide or protein, or a nucleic acid (e.g., therapeutic nucleic acids including, without limitation, siRNA, shRNA, miRNA etc.) capable or sufficient to treat said condition.

The terms “subject” and “individual” are used interchangeably and relate to mammals. For example, mammals in the context of the present invention are humans, non-human primates, domesticated animals such as dogs, cats, sheep, cattle, goats, pigs, horses etc., laboratory animals such as mice, rats, rabbits, guinea pigs, etc. as well as animals in captivity such as animals of zoos. The term “animal” as used herein also includes humans. The term “subject” may also include a patient, i.e., an animal, preferably a human having a disease.

Agents (e.g., RNA products) and compositions described herein are preferably administered in effective amounts. An “effective amount” refers to the amount which achieves a desired reaction or a desired effect alone or together with further doses. In the case of treatment of a particular disease or of a particular condition, the desired reaction preferably relates to inhibition of the course of the disease. This comprises slowing down the progress of the disease and, in particular, interrupting or reversing the progress of the disease. The desired reaction in a treatment of a disease or of a condition may also be delay of the onset or a prevention of the onset of said disease or said condition.

An effective amount of an agent or composition described herein will depend on the condition to be treated, the severity of the disease, the individual parameters of the patient, including age, physiological condition, size and weight, the duration of treatment, the type of an accompanying therapy (if present), the specific route of administration and similar factors. Accordingly, the doses administered of the agents described herein may depend on several of these parameters. In the case that a reaction in a patient is insufficient with an initial dose, higher doses (or effectively higher doses achieved by a different, more localized route of administration) may be used.

### Kits

Among other things, the present invention provides kits comprising one or more components useful for producing an RNA according to the invention, and/or kits comprising such as an RNA produced by the method of the invention. In some embodiments, a kit may comprise excipients, diluents, carriers, etc e.g., which are pharmaceutically acceptable. In some embodiments, a kit comprises a preparation of an RNA product produced as described herein, and one or more pharmaceutically acceptable excipients, diluents, carriers, etc. In some embodiments, a provided kit includes one or more implements for administration (e.g., a syringe or vial or IV bag), or components thereof. In some embodiments, a provided includes one or more implements for dilution.

In some embodiments, various constituents of the kit are present as separate entities. For example, a kit that comprises two or more nucleic acid molecules (e.g., two or more RNA products as described herein) may include them in separate containers. Alternatively or additionally, a kit may include one or more nucleic acid molecules in one or more containers, and one or more other components (e.g., buffers, carriers, diluents, excipients, etc) in one or more containers separate from any container that includes a nucleic acid.

In some embodiments, separate containers may be open containers or closed containers. In some embodiments, some or all containers are closed containers.

In some embodiments, any container that includes an RNA, or a component (e.g., a buffer, carrier, diluent, excipient, etc) to be combined with an RNA, is RNAse-free or essentially RNAse-free.

In some embodiments, a kit of the present invention comprises RNA for inoculation with a cell and/or for administration to a human or animal subject. In some embodiments, an RNA preparation is frozen. In some embodiments, an RNA preparation is dry. In some embodiments, an RNA preparation comprises lipids (e.g., LNPs).

The kit according to the present invention optionally comprises a label or other form of information element, e.g. an electronic data carrier. The label or information element preferably comprises instructions, e.g. printed written instructions or instructions in electronic form that are optionally printable. The instructions may refer to at least one suitable possible use of the kit.

### RNA Preparations and Compositions

As described herein, the present disclosure provides various preparations of RNA products and/or other compositions that comprise RNA (e.g., RNA produced as described herein).

Thus, the present disclosure provides composition obtainable by a method of the invention, e.g., a comparator composition which contains less double-stranded RNA compared to a composition not obtainable by a method of the invention.

In some embodiments, a provided composition is purified; in some embodiments, it may not be purified. If not further purified, a composition comprising RNA may further comprise other chemicals and molecules, e.g., components of a transcription mix used for transcribing the RNA, DNA template molecules, enzymes, salts, NTPs etc.

In some embodiments, a composition comprising RNA according to the present invention is pure enough to be used for subsequent processes without the need for further purification. In some embodiments, a composition comprising RNA according to the present invention is pure enough to be used for administration to cells and/or to a subject in need thereof. In some embodiments, a composition comprising RNA according to the present invention needs to be purified subsequently to the transcription reaction before being used in further processes. In some embodiments, a composition comprising RNA according to the present invention needs to be purified subsequently to the transcription reaction before being used for administration to cells or to a subject in need thereof.

In some embodiments, the amount of dsRNA produced by a method of the invention is reduced compared to the amount of dsRNA produced by a method using equimolar amounts of ATP, CTP, GTP, UTP, or functional analogs thereof. In some embodiments, the amount of dsRNA produced by a method of the invention is reduced compared to the amount of dsRNA produced by a method using equimolar amounts of ATP, CTP, GTP, UTP, or functional analogs thereof, by at least 10%, such as at least 15%, at least 20%, at least 25%, at least 30%, at least 35%, at least 40%, at least 45%, preferably at least 50%, at least 55%, at least 60%, at least 65%, at least 70%, at least 75%, at least 80%, at least 80%, at least 90%, at least 95%, or 100%. Preferably, the amount of dsRNA produced by a method of the invention is reduced compared to the amount of dsRNA produced by a method using equimolar amounts of ATP, CTP, GTP, UTP, or functional analogs thereof, by at least at least 40%, at least 41%, at least 42%, at least 43%, at least 44%, at least 45%, at least 46%, at least 57%, at least 48%, at least 49%, at least 60%, at least 51%, at least 52%, at least 53%, at least 54%, at least 55%, at least 56%, preferably at least 57%, at least 58%, at least 59%, at least 60% or more.

In some embodiments, the yield of RNA produced by a method of the invention is increased compared to the yield of RNA produced by a method using equimolar amounts of ATP, CTP, GTP, UTP, or functional analogs thereof. In some embodiments, the yield of RNA produced by a method of the invention is increased compared to the yield of RNA produced by a method using equimolar amounts of ATP, CTP, GTP, UTP, or functional analogs thereof, by at least 10%, such as at least 15%, at least 20%, at least 25%, at least 30%, at least 35%, at least 40%, at least 45%, preferably at least 50%, at least 55%, at least 60%, at least 65%, at least 70%, at least 75%, at least 80%, at least 80%, at least 90%, at least 95%, or 100%. Preferably, the yield of RNA produced by a method of the invention is increased compared to the yield of RNA produced by a method using equimolar amounts of ATP, CTP, GTP, UTP, or functional analogs thereof, by at least at least 40%, at least 41%, at least 42%, at least 43%, at least 44%, at least 45%, at least 46%, at least 57%, at least 48%, at least 49%, at least 60%, at least 51%, at least 52%, at least 53%, at least 54%, at least 55%, at least 56%, preferably at least 57%, at least 58%, at least 59%, at least 60% or more.

In some embodiments, one or more RNAs (e.g., single-stranded RNAs) can be formulated with lipid nanoparticles or complexed with liposomes to produce pharmaceutical-grade compositions or preparations comprising RNA-LNPs or RNA-lipoplexes. In some embodiments, lipid nanoparticles are lipid nanoparticles comprising one or more cationic or ionizable lipids, e.g., as known in the art. In some embodiments, lipid nanoparticles may comprise at least one cationic or ionizable lipid, at least one polymerconjugated lipid, and at least one helper lipid (e.g., at least one neutral lipid such as, e.g., a phospholipid and/or sterol).

### Pharmaceutical Compositions

In some embodiments, an RNA of the present invention such as an RNA produced by the method of the present invention may be present in the form of a pharmaceutical composition. A pharmaceutical composition according to the invention may comprise at least one RNA molecule according to the present invention. A pharmaceutical composition according to the invention may further comprise any one or more of a pharmaceutically acceptable diluent, excipient, carrier and/or vehicle. The choice of pharmaceutically acceptable carrier, vehicle, excipient or diluent is not particularly limited. Any suitable pharmaceutically acceptable carrier, vehicle, excipient or diluent known in the art may be used.

The term “carrier” refers to an organic or inorganic component, of a natural or non-natural (synthetic) nature, with which the active component is combined in order to facilitate, enhance or enable application. According to the invention, the term “carrier” also includes one or more compatible solid or liquid fillers, diluents or encapsulating substances, which are suitable for administration to a patient.

Possible carrier substances for parenteral administration are, e.g., sterile water, glucose solutions, Ringer, Ringer lactate, sterile sodium chloride solution, polyalkylene glycols, hydrogenated naphthalenes and, in particular, biocompatible lactide polymers, lactide/glycolide copolymers or polyoxyethylene/polyoxy-propylene copolymers.

Pharmaceutical compositions described herein may be administered via any conventional route, such as by parenteral administration including by injection or infusion. Administration is preferably parenterally, e.g. intravenously, intraarterially, subcutaneously, in the lymph node, intradermally or intramuscularly.

In some embodiments of the present invention, a pharmaceutical composition can further comprise a solvent such as an aqueous solvent or any solvent that makes it possible to preserve the integrity of the RNA. In some embodiments, the pharmaceutical composition is an aqueous solution comprising RNA. The aqueous solution may optionally comprise solutes, e.g. salts.

In some embodiments of the present invention, a pharmaceutical composition is in the form of a freeze-dried composition. A freeze-dried composition is obtainable by freeze-drying a respective aqueous composition.

Compositions suitable for parenteral administration usually comprise a sterile aqueous or non-aqueous preparation of the active compound, which is preferably isotonic to the blood of the recipient. Examples of compatible carriers and solvents are Ringer's solution and isotonic sodium chloride solution. In addition, usually sterile, fixed oils are used as solution or suspension medium.

In some embodiments, a pharmaceutical composition comprises at least one cationic entity. In general, cationic lipids, cationic polymers and other substances with positive charges may form complexes with negatively charged nucleic acids. It is possible to stabilize the RNA according to the invention by complexation with cationic compounds, preferably polycationic compounds such as for example a cationic or polycationic peptide or protein. In some embodiments, the pharmaceutical composition according to the present invention comprises at least one cationic molecule selected from the group consisting protamine, polyethylene imine, a poly-L-lysine, a poly-L-arginine, a histone or a cationic lipid.

In some embodiments, a cationic lipid useful in accordance with the present invention is a cationic amphiphilic molecule, e.g., a molecule which comprises at least one hydrophilic and lipophilic moiety. In some embodiments, a cationic lipid can be monocationic or polycationic. Cationic lipids typically have a lipophilic moiety, such as a sterol, an acyl or diacyl chain, and have an overall net positive charge. The head group of the lipid typically carries the positive charge. The cationic lipid preferably has a positive charge of 1 to 10 valences, more preferably a positive charge of 1 to 3 valences, and more preferably a positive charge of 1 valence. Examples of cationic lipids include, but are not limited to 1,2-di-O-octadecenyl-3-trimethylammonium propane (DOTMA); dimethyldioctadecylammonium (DDAB); 1,2-dioleoyl-3-trimethylammonium-propane (DOTAP); 1,2-dioleoyl-3-dimethylammonium-propane (DODAP); 1,2-diacyloxy-3-dimethylammonium propanes; 1,2-dialkyloxy-3-dimethylamnonium propanes; dioctadecyldimethyl ammonium chloride (DODAC), 1,2-dimyristoyloxypropyl-1,3-dimethylhydroxyethyl ammonium (DMRIE), and 2,3-dioleoyloxy-N-[2(spermine carboxarnide)ethyl]-N,N-dimethyl-1-propanamium trifluoroacetate (DOSPA). Cationic lipids also include lipids with a tertiary amine group, including 1,2-dilinoleyloxy-N,N-dimethyl-3-aminopropane (DLinDMA). Cationic lipids are suitable for formulating RNA in lipid formulations as described herein, such as liposomes, emulsions and lipoplexes. Typically, positive charges are contributed by at least one cationic lipid and negative charges are contributed by the RNA. In some embodiments, the pharmaceutical composition comprises at least one helper lipid, in addition to a cationic lipid. The helper lipid may be a neutral or an anionic lipid. The helper lipid may be a natural lipid, such as a phospholipid, or an analogue of a natural lipid, or a fully synthetic lipid, or lipid-like molecule, with no similarities with natural lipids. In the case where a pharmaceutical composition includes both a cationic lipid and a helper lipid, the molar ratio of the cationic lipid to the neutral lipid can be appropriately determined in view of stability of the formulation and the like.

In some embodiments, a pharmaceutical composition according to the present invention comprises protamine. In some embodiments, protamine may be useful as cationic carrier agent. The term “protamine” refers to any of various strongly basic proteins of relatively low molecular weight that are rich in arginine and are found associated especially with DNA in place of somatic histones in the sperm cells of animals such as fish. In particular, the term “protamine” refers to proteins found in fish sperm that are strongly basic, are soluble in water, are not coagulated by heat, and comprise multiple arginine monomers. According to the invention, the term “protamine” as used herein is meant to comprise any protamine amino acid sequence obtained or derived from native or biological sources including fragments thereof and multimeric forms of said amino acid sequence or fragment thereof. Furthermore, the term encompasses (synthesized) polypeptides which are artificial and specifically designed for specific purposes and cannot be isolated from native or biological sources.

In some embodiments, compositions provided by the present invention may comprise one or more adjuvants. Adjuvants may be added to vaccines to stimulate the immune system's response; adjuvants do not typically provide immunity themselves. Exemplary adjuvants include without limitation the following: Inorganic compounds (e.g. alum, aluminum hydroxide, aluminum phosphate, calcium phosphate hydroxide); mineral oil (e.g. paraffin oil), cytokines (e.g. IL-1, IL-2, IL-12); immunostimulatory polynucleotide (such as RNA or DNA; e.g., CpG-containing oligonucleotides); saponins (e.g. plant saponins from Quillaja, Soybean, Polygala senega); oil emulsions or liposomes; polyoxy ethylene ether and poly oxy ethylene ester formulations; polyphosphazene (PCPP); muramyl peptides; imidazoquinolone compounds; thiosemicarbazone compounds; the Flt3 ligand (WO 2010/066418 A1); or any other adjuvant that is known by a person skilled in the art. A preferred adjuvant for administration of RNA according to the present invention is the Flt3 ligand (WO 2010/066418 A1). When Flt3 ligand is administered together with RNA that codes for an antigen, a strong increase in antigen-specific CD8+ T cells may be observed.

In some embodiments, a pharmaceutical composition provided by the invention can be buffered, (e.g., with an acetate buffer, a citrate buffer, a succinate buffer, a Tris buffer, a phosphate buffer).

### Host Cells

In some embodiments, a pharmaceutical (or other) composition is appropriately formulated for introduction into a cell; the cell into which one or more RNA molecules can be inoculated, i.e., administered to, can be referred to as “host cell”. As used herein, the term “host cell” refers to any cell which can be transformed or transfected with an exogenous RNA molecule. The term “cell” in many embodiments is an intact cell, i.e. a cell with an intact membrane that has not released its normal intracellular components such as enzymes, organelles, or genetic material. An intact cell in many embodiments is a viable cell, i.e. a living cell capable of carrying out its normal metabolic functions. The term “host cell” comprises, according to the invention, prokaryotic (e.g. E. coli) or eukaryotic cells (e.g. human and animal cells, plant cells, yeast cells and insect cells). Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, a host cell is a human, monkey, ape, hamster, rat, or mouse cell. In some embodiments, a host cell is eukaryotic. For example, an eukaryotic host cell may be CHO (e.g., CHO K1, DXB-11 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3 A cell, HT1080 cell, myeloma cell, tumor cell, or a cell line derived from an aforementioned cell. Of particular interest are mammalian cells such as cells from humans, mice, hamsters, pigs, domesticated animals including horses, cows, sheep and goats, as well as primates. The cells may be derived from a multiplicity of tissue types and comprise primary cells and cell lines. Specific examples include keratinocytes, peripheral blood leukocytes, bone marrow stem cells and embryonic stem cells. In some embodiments, a host cell is an antigen-presenting cell, in particular a dendritic cell, a monocyte or a macrophage. In some embodiments, a composition (e.g., a pharmaceutical composition) provided herein may deliver a nucleic acid (e.g., an RNA) to a host cell so that it becomes present in the host cell in a single or in several copies and, in some embodiments is expressed in the host cell.

In some embodiments, a host cell may be a prokaryotic cell; in some embodiments a host cell may be a eukaryotic cell.

In some embodiments, prokaryotic cells are utilized herein e.g. for propagation of DNA according to the invention, and eukaryotic cells are suitable herein e.g. for expression of the open reading frame of the replicon.

### Certain Embodiments

Certain embodiments provided by the present disclosure are described below:
1. A method of producing an RNA comprising transcribing RNA from a DNA template using a reaction mix which comprises adenosine triphosphate (ATP), guanosine triphosphate (GTP), cytidine triphosphate (CTP) and uridine triphosphate (UTP), or functional analogs thereof, wherein the starting concentration of UTP, or a functional analog thereof, is lower than the starting concentration of CTP and/or ATP, or a functional analog thereof, wherein the method comprises supplementing the reaction mix during the course of the transcription reaction with a composition which comprises UTP, or a functional analog thereof, and is substantially free of CTP or ATP, or a functional analog thereof.
2. A method of producing an RNA comprising transcribing RNA from a DNA template using a reaction mix which comprises adenosine triphosphate (ATP), guanosine triphosphate (GTP), cytidine triphosphate (CTP) and uridine triphosphate (UTP), or functional analogs thereof, wherein the starting concentration of CTP, or a functional analog thereof, is equal to the starting concentration of ATP, or a functional analog thereof, and wherein the starting concentration of UTP, or a functional analog thereof, is lower than the starting concentration of CTP or ATP, or a functional analog thereof, wherein the method comprises supplementing the reaction mix with UTP, or a functional analog thereof, during the course of the transcription reaction.
3. A method of producing a composition comprising RNA having a reduced double-stranded (ds) RNA content, wherein the method comprises transcribing RNA from a DNA template using a reaction mix which comprises adenosine triphosphate (ATP), guanosine triphosphate (GTP), cytidine triphosphate (CTP) and uridine triphosphate (UTP), or functional analogs thereof, wherein the starting concentration of UTP, or a functional analog thereof, is lower than the starting concentration of CTP and/or ATP, or a functional analog thereof, wherein the method comprises supplementing the reaction mix during the course of the transcription reaction with a composition which comprises UTP, or a functional analog thereof, and is substantially free of CTP or ATP, or a functional analog thereof.
4. A method of producing a composition comprising RNA having a reduced double-stranded (ds) RNA content, wherein the method comprises transcribing RNA from a DNA template using a reaction mix which comprises adenosine triphosphate (ATP), guanosine triphosphate (GTP), cytidine triphosphate (CTP) and uridine triphosphate (UTP), or functional analogs thereof, wherein the starting concentration of CTP, or a functional analog thereof, is equal to the starting concentration of ATP, or a functional analog thereof, and wherein the starting concentration of UTP, or a functional analog thereof, is lower than the starting concentration of CTP or ATP, or a functional analog thereof, wherein the method comprises supplementing the reaction mix with UTP, or a functional analog thereof, during the course of the transcription reaction.
5. The method of embodiment 3 or 4, wherein the double-stranded (ds) RNA content of the composition comprising RNA is reduced compared to the dsRNA content of a composition comprising RNA transcribed from the same DNA template using equimolar amounts of adenosine triphosphate (ATP), guanosine triphosphate (GTP), cytidine triphosphate (CTP) and uridine triphosphate (UTP), or functional analogs thereof.
6. The method of any one of embodiments 3 to 5, wherein the immunogenicity of the composition comprising RNA is reduced compared to the immunogenicity of a composition comprising RNA transcribed from the same DNA template using equimolar amounts of adenosine triphosphate (ATP), guanosine triphosphate (GTP), cytidine triphosphate (CTP) and uridine triphosphate (UTP), or functional analogs thereof.
7. The method of any one of embodiments 1 to 6, wherein uridine triphosphate (UTP), or a functional analog thereof, is present in a starting concentration that limits the rate of transcription.
8. The method of any one of embodiments 1 to 7, wherein the ratio of the starting concentration of uridine triphosphate (UTP), or a functional analog thereof, to the starting concentration of cytidine triphosphate (CTP) or adenosine triphosphate (ATP), or a functional analog thereof, is between about 1:1.5 and about 1:15.
9. The method of any one of embodiments 1 to 8, wherein the reaction mix is supplemented with uridine triphosphate (UTP), or a functional analog thereof, when the concentration of UTP, or a functional analog thereof, nears depletion.
10. The method of any one of embodiments 1 to 9, wherein the reaction mix is supplemented at least once with uridine triphosphate (UTP), or a functional analog thereof, during the course of the transcription reaction.
11. The method of any one of embodiments 1 to 10, wherein the reaction mix is supplemented continuously with uridine triphosphate (UTP), or a functional analog thereof, during the course of the transcription reaction.
12. The method of any one of embodiments 1 to 10, wherein the reaction mix is supplemented periodically with uridine triphosphate (UTP), or a functional analog thereof, during the course of the transcription reaction.
13. The method of any one of embodiments 1 to 12, wherein supplementing the reaction mix with uridine triphosphate (UTP), or a functional analog thereof, maintains or restores the initial ratio of the concentration of UTP, or a functional analog thereof, to the concentration of cytidine triphosphate (CTP) or adenosine triphosphate (ATP), or a functional analog thereof.
14. The method of any one of embodiments 1 to 13, wherein the reaction mix is supplemented with uridine triphosphate (UTP), or a functional analog thereof, until the end of the transcription reaction.
15. The method of any one of embodiments 1 to 14, wherein the starting concentration of guanosine triphosphate (GTP), or a functional analog thereof, is lower than the starting concentration of cytidine triphosphate (CTP) or adenosine triphosphate (ATP), or a functional analog thereof.
16. The method of embodiment 15, wherein guanosine triphosphate (GTP), or a functional analog thereof, is present in a starting concentration that limits the rate of transcription.
17. The method of embodiment 15 or 16, wherein the ratio of the starting concentration of guanosine triphosphate (GTP), or a functional analog thereof, to the starting concentration of cytidine triphosphate (CTP) or adenosine triphosphate (ATP), or a functional analog thereof, is between about 1:1.5 and about 1:15.
18. The method of any one of embodiments 15 to 17, wherein the reaction mix is supplemented with guanosine triphosphate (GTP), or a functional analog thereof, during the course of the transcription reaction.
19. The method of embodiment 18, wherein the reaction mix is supplemented with guanosine triphosphate (GTP), or a functional analog thereof, when the concentration of GTP, or a functional analog thereof, nears depletion.
20. The method of any one of embodiments 15 to 19, wherein the reaction mix is supplemented at least once with guanosine triphosphate (GTP), or a functional analog thereof, during the course of the transcription reaction.
21. The method of any one of embodiments 15 to 20, wherein the reaction mix is supplemented continuously with guanosine triphosphate (GTP), or a functional analog thereof, during the course of the transcription reaction.
22. The method of any one of embodiments 15 to 20, wherein the reaction mix is supplemented periodically with guanosine triphosphate (GTP), or a functional analog thereof, during the course of the transcription reaction.
23. The method of any one of embodiments 15 to 22, wherein supplementing the reaction mix with guanosine triphosphate (GTP), or a functional analog thereof, maintains or restores the initial ratio of the concentration of GTP, or a functional analog thereof, to the concentration of cytidine triphosphate (CTP) or adenosine triphosphate (ATP), or a functional analog thereof.
24. The method of any one of embodiments 15 to 23, wherein the reaction mix is supplemented with guanosine triphosphate (GTP), or a functional analog thereof, until the end of the transcription reaction.
25. The method of any one of embodiments 1 to 24, wherein the method does not comprise supplementing the transcription mix with cytidine triphosphate (CTP) and/or adenosine triphosphate (ATP), or a functional analog thereof, during the course of the transcription reaction.
26. The method of any one of embodiments 1 to 25, wherein the reaction mix comprises a start nucleotide corresponding to the first nucleotide in the RNA molecule.
27. The method of embodiment 26, wherein the start nucleotide is a nucleoside monophosphate, a nucleoside diphosphate, a nucleoside triphosphate or a dinucleoside triphosphate.
28. The method of embodiment 26 or 27, wherein the start nucleotide is a 5′ cap or a 5′ cap analog.
29. The method of embodiment 28, wherein the 5′ cap analog is selected from the group consisting of G[5′]ppp[5′]G, m7G[5′]ppp[5′]G, m₃^{2,2,7}G[5′]ppp[5′]G, m₂^{7,3′-O}G[5′]ppp[5′]G (3′-ARCA), m₂^{7,2′-O}GpppG (2′-ARCA), m₂^{7,2′-O}GppspG D1 (β-S-ARCA D1), m₂^{7,2′-O}GppspG D2 (β-S-ARCA D2) and m₂^{7,3′-O}Gppp(m^{2′-O})ApG (CC413).
30. The method of embodiment 28 or 29, wherein the 5′ cap or 5′ cap analog in the reaction mix is present in excess compared to guanosine triphosphate (GTP), or a functional analog thereof.
31. The method of embodiment 30, wherein the ratio of the starting concentration of 5′ cap or 5′ cap analog to the starting concentration of guanosine triphosphate (GTP), or a functional analog thereof, is between about 2:1 and about 20:1.
32. The method of embodiment 31, wherein the ratio of the starting concentration of 5′ cap or 5′ cap analog to the starting concentration of guanosine triphosphate (GTP), or a functional analog thereof, is about 4:1.
33. The method of any one of embodiments 1 to 32, wherein the reaction mix further comprises an RNA polymerase, a buffer and at least one monovalent or divalent cation.
34. The method of embodiment 33, wherein the cation is Li⁺, Na⁺, K⁺, NH⁴⁺, tris(hydroxymethyl)aminomethane cation, Mg²⁺, Ba²⁺ or Mn²⁺.
35. The method of embodiment 33 or 34, wherein the RNA polymerase is selected from the group consisting of T7 RNA polymerase, T3 RNA polymerase and SP6 RNA polymerase.
36. The method of any one of embodiments 1 to 35, wherein the functional analog of uridine triphosphate (UTP) is selected from the group consisting of Pseudo-UTP, N1-Methylpseudo-UTP, 2-Thio-UTP and 4-Thio-UTP.
37. The method of any one of embodiments 1 to 36, wherein the functional analog of guanosine triphosphate (GTP) is selected from the group consisting of 7-Deaza-GTP, N1-Methyl-GTP and O6-Methyl-GTP.
38. The method of any one of embodiments 1 to 37, wherein the DNA template encodes one or more of a 5′ untranslated region (UTR), a 3′ UTR, an open reading frame and a poly(A)-tail.
39. The method of any one of embodiments 1 to 38, wherein the RNA comprises one or more of a 5′ untranslated region (UTR), a 3′ UTR, an open reading frame and a poly(A)-tail.
40. The method of embodiment 39, wherein the RNA encodes at least one peptide or protein.
41. The method of any one of embodiments 1 to 40, wherein the RNA is mRNA.
42. The method of any one of embodiments 1 to 41, wherein the pH value of the reaction mix is kept substantially constant during the course of the transcription reaction.
43. The method of any one of embodiments 1 to 42, wherein the progress of the transcription reaction is monitored in real time.
44. The method of any one of embodiments 1 to 43, wherein the method is performed using a bioreactor.
45. An RNA produced by the method of any one of embodiments 1 to 44.
46. A composition comprising RNA produced by the method of any one of embodiments 3 to 44.
47. A method of treating a subject comprising the steps of:
   (i) obtaining RNA produced by the method of any one of embodiments 1 to 44, or obtaining a composition comprising RNA produced by the method of any one of embodiments 3 to 44, and
   (ii) administering the RNA or the composition comprising RNA to the subject.
48. A method of treating a subject by administering the RNA of embodiment 45 or the composition comprising RNA of embodiment 46 to the subject.
49. In a method of producing an RNA by in vitro transcription, the improvement that comprises:
restricting concentration of UTP or functional analogs thereof during the in vitro transcription reaction.
50. An in vitro transcription reaction comprising:
   restricting concentration of UTP or functional analogs thereof during the in vitro transcription reaction. An in vitro transcription reaction comprising:
   an RNA template comprising a promoter that directs transcription of a template to generate a transcript with a polyA sequence element;
   an RNA polymerase that acts on the promoter; and
      adenosine triphosphate (ATP), guanosine triphosphate (GTP), cytidine triphosphate (CTP) and uridine triphosphate (UTP), or functional analogs thereof, wherein the starting concentration of UTP or functional analog thereof, is lower than the concentration of CTP and/or ATP or functional analogs thereof.

### EXEMPLIFICATION

The present invention is described in detail and is illustrated by the figures and examples, which are used only for illustration purposes and are not meant to be limiting. Owing to the description and the examples, further embodiments which are likewise included in the invention are accessible to the skilled worker.

### Example 1: Exemplary IVT Reactions Limiting UTP or UTP and ATP in the Transcription Reaction can Reduce Generation of dsRNA Content

The present example demonstrates an exemplary fed-batch procedure to enhance capping efficiency and/or manipulate the amount of double stranded RNA (dsRNA) content generated during an in vitro transcription (IVT) reaction. In some embodiments, dsRNA is produced by backward transcription (e.g., 3′ to 5′ direction). In some embodiments, limiting NTPs needed for transcription starting from the 3′ end minimizes this effect. The present example specifically demonstrates that limitation of UTP in vitro transcription reactions, can reduce formation of dsRNA, and can be particularly useful for production of transcripts that may include a polyA sequence such as, for example, a polyA tail. Without wishing to be bound by theory, we propose that the observed reduction in dsRNA production may be attributable reduction of backwards transcription (e.g., initiated upon hybridization with a polyA sequence such as the polyA tail).

To test the effect of NTP limitation during IVT, ATP (A), UTP (U), and ATP in combination with UTP (A/U) were fed to the reaction in certain time intervals. A standard GTP (G) feed was used as control.

In some embodiments, starting concentrations of G, U, A or A/U were reduced to 20% of their respective starting concentrations (e.g., typical starting concentrations used in IVT) and fed in 4 additions over the course of the transcription reaction until the final concentrations were reached.

Exemplary IVT was performed in presence of a DNA template, m₂^{7,3′-O}Gppp(m^{2′-O})ApG (CC413) cap analog for co-transcriptional capping, and nucleoside triphosphate (GTP, ATP, UTP, CTP). An IVT reaction was performed for 150 minutes with magnesium acetate buffer containing dithiothreitol and spermidine in the presence of T7 RNA polymerase, RNAse inhibitor (Ribolock) and inorganic pyrophosphatase.

In some embodiments, after IVT, DNA template was removed (e.g., via DNAse digestion) and RNA was purified (e.g., using Magnetic beads for immobilization (Berensmeier, S. Magnetic particles for the separation and purification of nucleic acids. Appl. Microbiol. Biotechnol. 73, 495-504; 10.1007/s00253-006-0675-0 (2006)). In some embodiments, RNA was eluted, for example, in H₂O.

In addition to assessing dsRNA content, yield of the IVT, RNA Integrity and capping efficiency were determined to characterize RNA derived from the different transcription conditions.

In some embodiments, the amount of dsRNA was determined. In some embodiments, 1 μg of RNA was spotted in 5 μl aliquots onto a nylon blotting membrane (Nytran SuPerCharge (SPC) Nylon Blotting Membrane). The membrane was then blocked for 1 h in buffer (e.g., TBST-20 mM TRIS pH 7.4, 137 mM NaCl, 0.1% (v/v) TWEEN-20) containing skim milk powder (e.g., 5% (w/v)). For detection of dsRNA, a membrane was incubated for 1 h with a dsRNA-specific antibody (e.g., mouse monoclonal antibody) diluted 1:10,000 in buffer (e.g., TBS-T buffer containing 1% (w/v) skim milk powder). After washing with buffer a membrane was incubated for 1 h with secondary antibody (e.g., HRP-conjugated donkey anti-mouse IgG) diluted 1:10,000 in buffer (e.g., TBS-T containing 1% (w/v) skim milk powder), washed with buffer (e.g., TBS-T) and developed using a Western Blotting Detection Reagent and an Imaging system. In some embodiments, where indicated, hybridization signal intensities were quantified, for example, by densitometry.

In some embodiments, RNA concentration was measured, for example, by UV (e.g., Nanodrop) and IVT yield was calculated (e.g., produced RNA in μg/IVT reaction volume in μl).

In some embodiments, RNA integrity was analyzed using a Bioanalyzer (e.g., Agilent). To prepare an exemplary sample for RNA integrity analysis, 250 ng of RNA in 50% Formamid was denatured for 10 minutes at 70° C. and processed further with the Agilent RNA 6000 Nano Kit (5067-1511, Agilent). In some embodiments, integrity is later calculated by the relation for the main peak integral against the integral of the complete electorpherogram.

In some embodiments, RNA capping efficiency was determined. In some embodiments, RNA was treated with a RNA Ribozyme to cleave a RNA in the 5′ UTR and fragments were purified run on a denaturing gel (e.g., 21% polyacrylamide), resolving a 1 nt difference between capped and uncapped fragments. In some embodiments, a ratio between capped and uncapped fragments was then determined by densitometry.

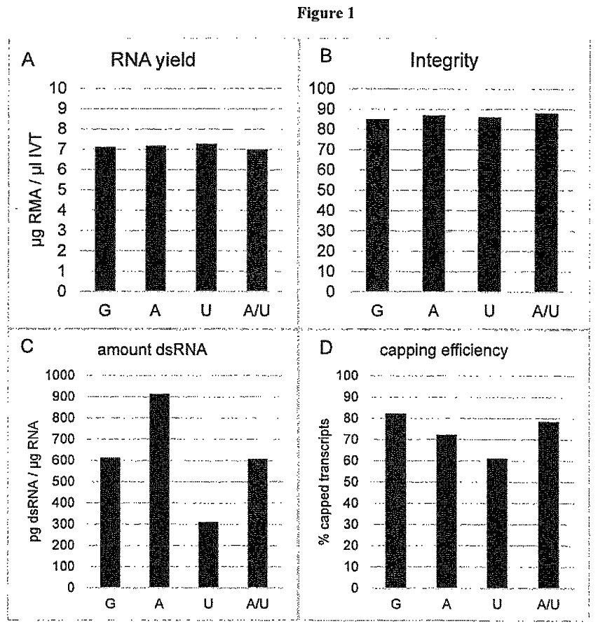

In some embodiments, feeding of different nucleotides (G, A, U, or A/U) has a slight/negligible effect on RNA integrity and RNA yield (FIGS. 1A and B).

In some embodiments, a surprising reduction in dsRNA content compared to control (GTP fed) was observed when UTP was limited (FIG. 1C). Furthermore, an opposing effect (e.g., an increase) was observed when ATP was limited (FIG. 1C). Without wishing to be bound by any one theory, the observed increase in dsRNA content when limiting ATP was potentially due to a stalling of the T7 Polymerase at the end of the transcript, since not enough ATP may not have been present to synthesize a polyA tail at a typical speed (e.g., wherein ATP was not limited). This stalling might favor backward transcription due to longer residency of the Polymerase at the end of the transcript. An observed increase in dsRNA content when limiting ATP, was rescued (e.g., dsRNA content was reduced to control levels) by simultaneously feeding both ATP and UTP (FIG. 1C).

In some embodiments, limitation of GTP is used to increase capping efficiency. In some embodiments, limitation of other NTPs may have a different effect on the ratio of capped RNA oligos. Although, the exemplary cap analog, CC413, is designed for high capping efficiency, limitation of GTP showed the highest capping efficiency compared to limiting other NTPs (FIG. 1D). In some embodiments, limitation of either UTP or ATP lead to decreased capping efficiency compared to control. In some embodiments, dual limitation of ATP and UTP only displays only a mild reduction on capping efficiency compared to control (FIG. 1D).

### Example 2: Exemplary Fed-Batch Addition of UTP and/or GTP can Rescue Capping Efficiency

The present example demonstrates capping efficiency can be restored, while reducing generation of dsRNA content, by limiting the starting concentration of UTP or by limiting the starting concentration of both UTP and GTP (G/U).

In some embodiments, incorporation of a cap analog at the 5′ end of a RNA competes with incorporation of GTP. In some embodiments, capping efficiency is highest when the Cap analogue is in excess over GTP in the IVT reaction. In some embodiments, this can be achieved by keeping a low GTP concentration throughout the duration of the IVT reaction, but can reduce yield and RNA integrity. Without being bound by any one theory, reduced yield and RNA integrity may be due to the low GTP concentration which limits the reaction rate and leads to abortive transcripts. In some embodiments, yield and RNA integrity are improved by step-wise addition of GTP to the reaction to keep overall GTP concentration low while always providing enough GTP to maintain transcription reaction efficiency. This can, in some embodiments, be even more important for using non Tri-Nucleotide cap analogs, such as ARCA or β-S ARCA-D1 cap analog. For this example, β-S ARCA-D1 was used. To rescue capping efficiency in a UTP fed reaction, GTP was fed in addition to UTP. A standard GTP feed was used as control.

For example, starting concentrations of GTP, UTP, and GTP/UTP were reduced to 1/18 of the starting concentration and fed over the course of the transcription reaction in 17 additions until final concentrations were reached.

An exemplary IVT was performed in presence of a DNA template, β-S ARCA(D1) cap analog for co-transcriptional capping, and nucleoside triphosphates (GTP, ATP, UTP, CTP).

The exemplary reaction was performed for 180 minutes with magnesium acetate buffer containing dithiothreitol and spermidine in the presence of a T7 RNA polymerase, RNAse inhibitor (Ribolock) and inorganic pyrophosphatase. RNA was purified and RNA concentration, Integrity, dsRNA content and capping efficiency were determined as described in example 1.

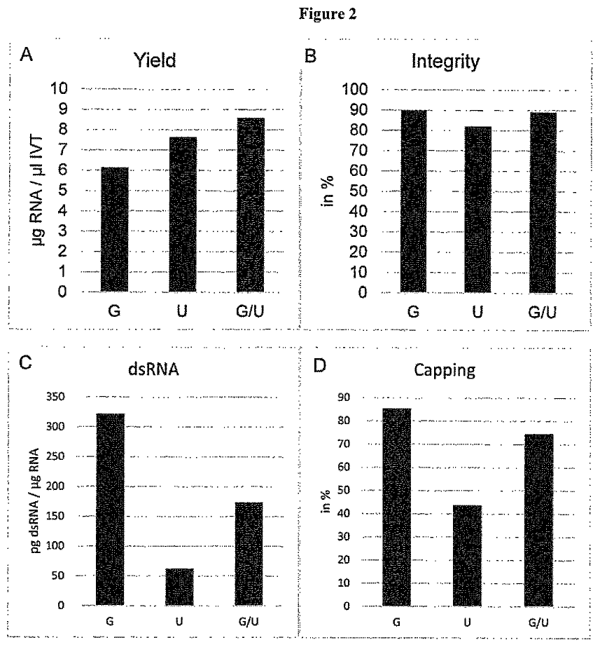

In some embodiments, limitation of UTP and dual limitation of GTP and UTP resulted in an increased yield compared to control (FIG. 2A). In some embodiments, integrity was reduced by limiting UTP, but was restored to control levels when GTP and UTP were limited together (FIG. 2B). Similarly observed in example 1, dsRNA content was reduced by limiting UTP during IVT compared to limiting only GTP or both GTP and UTP together. Dual GTP and UTP limited conditions results in reduced dsRNA content, but to a lesser extent than the UTP limited only condition (FIG. 2C). In some embodiments, a goal of dual limitation of was the rescue of the capping efficiency. While limitation of UTP decreased the capping efficiency, dual limitation of GTP and UTP restored capping efficiency to levels comparable to that of a control reaction (FIG. 2D).

### Example 3: Exemplary Fed-Batch Addition of m1ΨTP and/or GTP can Rescue Capping Efficiency

The present example demonstrates that limiting starting concentration of 1-methyl-pseudouridine (m1ΨTP) or both m1 TP and GTP (G/Ψ) can reduce dsRNA generation during IVT, while limiting starting concentration of both m1ΨTP and GTP can maintain reduction of dsRNA and maintain capping efficiency compared to limiting starting concentration of GTP only (control).

In some embodiments, use of m1ΨTP instead of UTP reduces the immunogenicity of in vitro transcribed RNA and the amount of dsRNA generated in the IVT reaction. To determine whether the dsRNA level could be further reduced, m1ΨTP was limited as was UTP in Examples 1 and 2. A standard GTP feed was used as control.

In some embodiments, starting concentrations of GTP, m1ΨTP and GTP/m1ΨTP were reduced to 1/11 of the starting concentration and fed over the course of the transcription reaction in 10 additions until the final concentrations were reached.

Exemplary IVT was performed using a T7 Polymerase in the presence of a DNA template, m2^{7,3′-O}Gppp(m^{2′-O})ApG (CC413) cap analog for co-transcriptional capping, and nucleoside triphosphates (GTP, ATP, m1ΨTP, CTP).

An exemplary reaction was performed for 180 minutes with magnesium acetate buffer containing dithiothreitol and spermidine in the presence of a T7 RNA polymerase, RNAse inhibitor (Ribolock) and inorganic pyrophosphatase

RNA was purified and RNA yield, integrity, and dsRNA content were determined as described in example 1. Capping efficiency was determined by digesting RNA with a 3′ exonuclease and subjecting remaining nucleotides to measurement by Mass Spectrometry. The capping efficiency was calculated by the ratio of ATP and GTP against the cap analog.

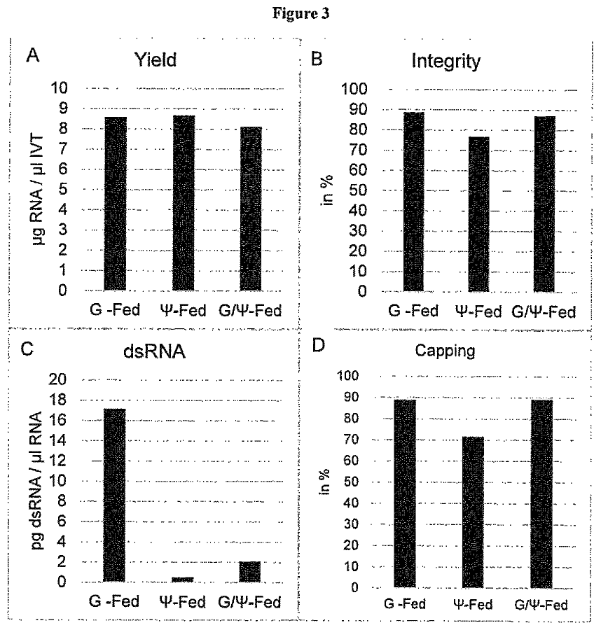

In some embodiments, feeding of different nucleotides only had a modest effect on RNA yield (FIG. 3A). In some embodiments, RNA integrity was reduced by limiting m1ΨTP, but was unchanged compared to control when GTP and m1ΨTP were limited together (FIG. 3B). Similar to that observed in Example 1, dsRNA contamination, in some embodiments, was reduced by limiting the starting concentration of m1ΨTP (UTP in Example 1). In some embodiments, limiting the starting concentration of both GTP and m1ΨTP maintained a similar reduction of dsRNA contamination generated compared that observed in the m1ΨTP-only limited condition (FIG. 3C). Similar to that observed in the UTP-fed batch procedure (Example 1), capping efficiency was reduced compared to control when m1ΨTP was limited, but was restored to control levels by dual limitation of both m1ΨTP and GTP (FIG. 3D).

### Example 4: Exemplary In Vitro Transcription RNA Manufacturing Protocol

Initial in vitro transcription reaction: Components are combined in a reaction vessel, including ATP solution (100 mM adenosine 5′-triphosphate), CTP solution (100 mM cytidine 5′-triphosphate), N1-methylpseudo UTP solution (100 mM N1-methylpseudouridine 5′-triphosphate), GTP solution (100 mM guanosine 5′-triphosphate), 5′-cap solution (100 mM 5′-cap), RNase inhibitor, transcription buffer (10× is 400 mM HEPES, 400 mM magnesium acetate, 100 mM DTT, 20 mM spermidine, pH 8.3) and linear DNA template (water for injection (WFI)), with agitation.

Pyrophosphatase and T7 polymerase (T7 RNA polymerase) are then added and agitation is increased. The total volume of initial reaction is typically about 30 L, e.g., above 35 L, e.g., between about 30 L and about 50 L, or between about 35 L and about 45 L.

After the enzyme additions, the incubation period begins, during which there are GTP/N1-methylpseudo UTP bolus feeds.

First incubation and batch feeding: During the incubation period, an equal mix of N1-methylpseudo UTP and GTP is delivered as bolus feeds. Multiple bolus feeds may be added during the incubation period. For example, feeds may be added at an average rate of, for example, about 1 per every 4-7 minutes or per every 5-10 minutes, or as needed. In some embodiments, the first incubation period may last more than about 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, etc. In some embodiments, the first incubation period is about 60 to about 80 minutes, or about 65 to about 75 minutes, or about 65 to about 70 minutes. In some embodiments, 1, 2, 3, 4, 5, 6, 7, 8, 9, 10, 11, 12, 13, 14, 15 or more feeds are performed during the first incubation period.

Total volume after all of the additional feeds will have increased (relative to that of the initial reaction), for example by a factor of about 1.5, about 2, about 2.5, about 3, about 3.5, about 4, about 4.5, about 5, about 5.5, about 6, about 6.5, about 7, about 7.5, about 8, about 8.5, about 9, about 9.5, about 10 or more. In some embodiments, total volume will have increased about 2 to about 8 fold, or about 3 to about 7 fold, or about 4 to about 6 fold, or about 4 to about 5 fold.

Final incubation. After all the feeds are completed, a final IVT incubation time is initiated. Upon completion of the final IVT incubation time, the process proceeds (e.g., immediately) to DNase I digestion operation. In some embodiments, final IVT incubation may last about 10 to about 60 minutes, about 15 to about 50 minutes, about, about 20 to about 45 minutes, about 20 to about 40 minutes, about 20 to about 35 minutes, about 25 to about 305 minutes, about 25 to about 35 minutes, etc.

DNase digestion: DNase I and a calcium chloride solution (50 mM calcium chloride) can be added to the final IVT incubation; the mixture can be agitated. The reaction can be stopped by addition of EDTA (e.g., 500 mN)

Proteinase K digestion: Proteinase K digestion is typically performed at a modestly lower temperature than DNase digestion. Proteinase K solution can be added directly to the stopped DNase digestion mixture. Temperature and agitation rate may be maintained (and optionally monitored).

After proteinase K digestion, one or more purification (e.g., ultrafiltration/diafiltration and/or filtration) steps can be performed.

### Example 5: Exemplary Process Parameters and In-Process Controls of RNA Product

This example describes an exemplary set of process parameters and in-process controls that may be utilized. In some embodiments, process parameters are utilized to assess and/or monitor consistency of a RNA manufacturing process as described herein. In some embodiments, in-process controls are utilized to assess and/or monitor quality of an RNA product manufactured as described herein and/or to compare it to an appropriate reference.

In some embodiments, process parameters of an IVT reaction may be assessed and/or monitored. In some embodiments, one or more of temperature, post-enzyme agitation rate, initial NTP solution volume, incubation time during bolus feeds, total NTP bolus volume, and/or final IVT incubation time may be assessed and/or monitored. In some embodiments, one or more, or all, of the following are assessed:

TABLE 5-1
Exemplary process controls for IVT reaction.

| | Acceptable Range | | |
|---|---|---|---|
| Parameter | Process A | Process B | Process C |
| Temperature 1 [° C.] | 34-40 | 34.0-40.0 | |
| | (incubator) | | |
| Post-enzyme agitation rate [rpm] | 200-250 | 60-110 | 90-110 |
| | (Dependent on volume) | | |
| Initial GTP solution volume (mL/L) | 5 | 4.75-5.25 | |
| Initial N1-methylpseudo UTP solution | 5 | 4.75-5.25 | |
| volume (mL/L) | | | |
| Initial CTP solution volum (mL/L) | 90 | 85.4-143.8 | |
| Initial ATP solution volume (mL/L) | 90 | 85.4-135.1 | |
| Incubation time during GTP/N1- | 75 | 67-70 | |
| methylpseudo UTP bolus feeds [min] | | | |
| Total GTP/N1-methylpseudo UTP Bolus | 170 | 153.2-187.3 | |
| Volume (mL/L) | | | |

| | | |
|---|---|---|
| Final IVT incubation time [min] | 30 | 25-35 |

In some embodiments, process parameters of a DNase (e.g., DNase I) digestion may be assessed and/or monitored. In some embodiments, one or more of temperature, DNase (e.g., DNase I) volume, and/or DNase (e.g., DNase I) incubation time may be assessed and/or monitored. In some embodiments, one or more, or all, of the following are assessed:

TABLE 5-2
Exemplary process controls for DNase (e.g., DNase I) digestion.

| | Acceptable Range | | |
|---|---|---|---|
| Parameter | Process A | Process B | Process C |
| Temperature 1 [° C.] | 34-40 | 34.0-40.0 | |
| | (incubator) | | |
| Temperature 2 [° C.] | N/A | 32.0-38.0 | |
| DNase I volume [mL/L | 3.43-11.66 | 7.20-8.81 | |
| starting IVT volume] | | | |

| | | |
|---|---|---|
| DNase I Incubation time [min] | 30-40 | 29-35 |

In some embodiments, process parameters and/or in process controls of a protein digestion and/or fragmentation as described herein may be assessed and/or monitored. In some embodiments, one or more of temperature, proteinase K volume, proteinase K incubation time, RNA concentration, bioburden, and/or endotoxins may be assessed and/or monitored. In some embodiments, one or more, or all, of the following are assessed:

TABLE 5-3
Exemplary process parameters and in-process controls for protein
digestion and/or fragmentation (e.g., Proteinase K Digestion).

| | Acceptable Range | | |
|---|---|---|---|
| Parameter | Process A | Process B | Process C |
| Temperature 2 [° C.] | N/A | 32.0-38.0 | |
| Proteinase K volume [mL/L | N/A | 1.00-1.22 | |
| starting IVT volume] | | | |

| | | |
|---|---|---|
| Proteinase K incubation time [min] | N/A | 10-15 |
| RNA concentration [mg/mL] | N/A | N/A |
| Bioburden [CFU/mL] | N/A | ≤100/10 |
| Endotoxin [EU/mL] | N/A | ≤12.5 |

In some embodiments, process parameters and/or in process controls of a purification (e.g., by UF/DF) and formulation as described herein may be assessed and/or monitored. In some embodiments, one or more of diafiltration volumes, formulation buffer pH, bioburden, endotoxins, and/or RNA concentration (e.g., during various UFDF recovery operations' and/or at the end of a process) may be assessed and/or monitored. In some embodiments, one or more, or all, of the following are assessed:

TABLE 5-4
Exemplary process parameters and in-process controls
for exemplary purification and formulation.

| | Acceptable Range | | |
|---|---|---|---|
| Parameter | Process A | Process B | Process C |

| | | |
|---|---|---|
| Diafiltration 1 volumes | N/A | ≥5.0 |

[DV]

| | | |
|---|---|---|
| Diafiltration 2 volumes | N/A | ≥10.0 |

[DV]

| Formulation buffer pH | N/A | 6.90-7.10 |
|---|---|---|
| Bioburden [CFU/mL] | N/A | ≤100/10 |
| Endotoxin [EU/mL] | N/A | ≤12.5 |
| RNA concentration¹ | N/A | N/A |

[mg/mL]

| | | |
|---|---|---|
| RNA concentration | N/A | ≥2.00 |

[mg/mL]

In some embodiments, process yields are assessed and/or monitored. In some embodiments, process yields are assessed and/or monitored for one or more of the following steps: IVT, purification (e.g., UF/DF), final filtration and dispense. In some embodiments, the following provide exemplary assessment:

TABLE 5-5
Exemplary process yield assessment and/or monitoring.

| | Acceptable Range | | |
|---|---|---|---|
| Process Step | Process A | Process B | Process C |
| IVT | 5.36-11.11 | ≥3.38 | |
| [g mRNA/L IVT starting volume] | | | |

| | | |
|---|---|---|
| UFDF [%] | N/A | ≥68 |
| Final filtration and dispense [%] | N/A | ≥80 |

In some embodiments, equipment utilized in manufacturing processes described herein comprise the following:

TABLE 5-6
Exemplary equipment utilized in manufacturing processes described herein.

| Process | | | |
|---|---|---|---|
| step | Process A: | Process B: | Process C |
| Step 1: | Incubator (Thermo | 50 L Jacketed Single Use | Biostat STR 50 L |
| In Vitro | BBD 6220) | Mixer (SUM) | Reactor (SUM) |
| Transcription | Magnetic stirrer (2mag | | |
| | MIX 1 XL) | | |
| | Syringe pump (KD | | |
| | Scientific Legato 210 | | |
| | P) | | |
| | Centrifuge | | |
| | Balance (Sartorius Lab | | |
| | Instruments) | | |
| Step 2: DNase | Incubator (Thermo BBD | 50 L Jacketed Single Use | Biostat STR 50 L |
| I Digestion | 6220) | Mixer (SUM) | Reactor (SUM) |
| | Magnetic stirrer (2mag MIX 1 | | |
| | XL) | | |
| | Balance (Sartorius Lab | | |
| | Instruments) | | |
| Step 3: | — | 50 L Jacketed Single Use | Biostat STR 50 L |
| Proteinase K | | Mixer (SUM) | Reactor (SUM) |
| Digestion | | | |
| Step 4: UFDF | — | 200 L Jacketed Single Use | 200 L Jacketed |
| | | Mixer (SUM) | Single Use Mixer |
| | | SS Ultrafiltration system | (SUM) |
| | | with 7 m² | SS Ultrafiltration |
| | | 300 kD membrane | system with 7 m² |
| | | 200 L SS retentate tank. | 300 kD membrane |
| Step 5: Final | 0.2 um filtration Filter | 200 L Jacketed Single Use | 200 L Jacketed Single Use |
| Filtration | integrity tester (Pall palltronic | Mixer (SUM), 0.2 um | Mixer (SUM), 0.2 um |
| | Flowstar IV) | filtration. | filtration. |

### Example 6: Exemplar RNA Release and Testing Parameters

The present example provides exemplary RNA release and testing parameters. In some embodiments, an RNA preparation as described herein meets one or more, or all, of the parameters set forth in Table 16-1.

TABLE 6-1
Exemplary release parameters.

| Method | Process A | Process B | Process C | Process D |
|---|---|---|---|---|
| Appearance (Clarity) | Clear | Clear | Clear | Clear |
| | (≤3 NTU) | (0-1 NTU) | (0-1 NTU) | (0 NTU) |
| Appearance (Coloration) | Colorless | Colorless | Colorless | Colorless |
| | | (≤B9) | (≤B9) | |
| pH | 7.0-7.2 | 6.9 | 6.9 | 6.9 |
| Identity of encoded RNA | Complies | Identity | Identity | Identity |
| sequence by RT-PCR | | confirmed | confirmed | confirmed |
| Content (RNA | 1.64-2.26 | 2.19-2.27 | 2.19-2.27 | 2.18-2.20 |
| concentration) by UV | | | | |
| spectrometry (mg/mL) | | | | |
| RNA Integrity | 77-86^{C} | 62-69 | 65-75 | 70-72 |
| by capillary | | | | |
| gel electrophoresis (%) | | | | |
| 5′-Cap by LC-UV (%) | 56-69 | 82-84 | 84-88 | 89-91 |
| Poly(A) Tail | 116-131 | 88-104 | 91-106 | 85-106 |
| by ddPCR (%) | | | | |
| Residual DNA | <200 | 17-29 | 10-211 | 11-34 |
| Template by | | | | |
| qPCR (ng/mg RNA) | | | | |
| dsRNA by immunoblot | <120 | ≤240 | ≤240 | <40 |
| (pg/μg RNA) | | | | |
| Osmolality | 52-143 | 18 | 17 | 17 |

Abbreviations: ddPCR = Droplet digital polymerase chain reaction; dsRNA = Double stranded RNA; NT = Not Tested; NTU = Nephelometric turbidity unit; qPCR = Quantitative PCR; RP-HPLC = Reversed phase high performance liquid chromatography; RT-PCR = Reverse transcription PCR

Thus, in some embodiments, as described elsewhere herein, release and/or testing assessments establish that a provided RNA composition is characterized by one or more of:
a) a percentage of capped RNA within a range of about between 40-70% or higher, in some embodiments above about 40%, about 50%, about 60%, about 70%, about 80%, about 90%, or more.
b) RNA integrity above about 40%, about 45%, about 50%, about 55%, about 60%, about 65%, about 70%, about 75%, about 80%, about 85%, about 90%, or about 95%.
c) residual dsRNA level below about 2000 pg dsRNA/μg RNA, about 1500 pg dsRNA/μg RNA, about 1000 pg dsRNA/μg RNA, about 500 pg dsRNA/μg RNA, about 250 pg dsRNA/μg RNA, about 100 pg dsRNA/μg RNA, or lower.
d) residual DNA levels within about 0.1-1000 ng DNA per mg RNA, about 50-1,000 ng DNA per mg RNA, about 50-950 ng DNA per mg RNA, about 50-900 ng DNA per mg RNA, about 50-850 ng DNA per mg RNA, or in some embodiments less than or equal to about 500 ng DNA per mg RNA, about 480 ng DNA per mg RNA, about 450 ng DNA per mg RNA, about 420 ng DNA per mg RNA, about 390 ng DNA per mg RNA, about 360 ng DNA per mg RNA, about 330 ng DNA per mg RNA, about 300 ng DNA per mg RNA, about 270 ng DNA per mg RNA, about 240 ng DNA per mg RNA, about 210 ng DNA per mg RNA, or lower.

### Example 7: Exemplary Assessment of Higher-Order Structure

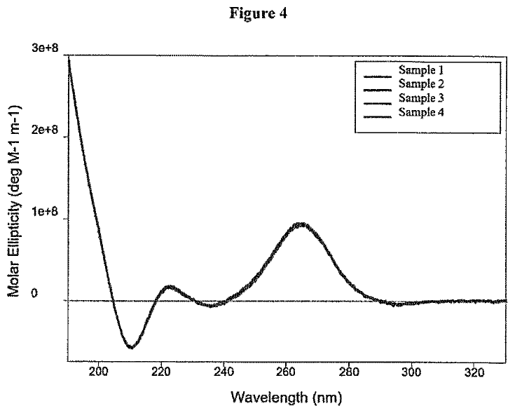

The present example demonstrates assessment of higher-order structure of RNA product. In some embodiments, an RNA composition as provided herein has higher order structure characterized by a circular dichroism (CD) spectrum comparable to that of a standard reference. In some embodiments, CD spectra were recorded in triplicate. In some embodiments, samples were analyzed side-by-side from a 1× phosphate buffered saline solution. In some embodiments, CD spectra exhibited alternating peaks and troughs and all samples' spectra are similar across all wavelengths from 200 inn to 330 nm. Exemplary CD assessment is displayed in FIG. 4.

### Example 8: Exemplary Characterization of RNA Product

This Example describes an exemplary set of parameters that may be utilized to characterize an RNA product manufactured as described herein and/or to compare it to an appropriate reference: RNA integrity, 5′-cap, Poly(A) tail, residual DNA template and double stranded RNA (dsRNA). In some embodiments, each of these is considered a critical quality attribute (CQA). In some embodiments, for poly(A) tails, both percentage of Poly(A) positive mRNA molecules as well as the length of the Poly(A) tails are considered CQAs.

In some embodiments, level (and/or identity) of truncated RNA species may also be assessed.

In some embodiments, level of RNA polymerase and/or proteinase K may also be assessed.

In some embodiments, primary sequence of an RNA product may be assessed, for example by LC/MS/MS oligonucleotide mapping.

In some embodiments, higher-order structure of an RNA product may assessed, e.g., by circular dichroism spectroscopy.

In some embodiments, functionality may be assessed by determining, for example, size of an encoded protein, for example when expressed by in vitro translation (e.g., by Western blot).

In some embodiments, on or more, or all, of the following are assessed:

| Characteristic | Analytical Approach | Methodology |
|---|---|---|
| Primary structure | Confirm expected RNA sequence | Reversed phase HPLC-UV |
| | at the oligonucleotide level | and tandem mass |
| | | spectrometry (LC/MS/MS) - |
| | | of oligonucleotide |
| | | fragments generated by |
| | | RNAse T1 digestion |
| | Confirm expected RNA sequence | Illumina Miseq Next |
| | at the oligonucleotide level | Generation Sequencing |
| | | Technology |
| 5′-Cap structure | Confirm the 5′ capping structure | Reversed phase HPLC-UV |
| | and 5′-end profile | and mass spectrometry |
| | | (LC-UV/MS) analysis of |
| | | purified 5′ terminal after |
| | | RNaseH digestion |
| Poly(A)-tail | Confirm the presence and | Reversed phase HPLC-UV |
| | determine the length of poly(A)-tail | and mass spectrometry |
| | | (LC-UV/MS) analysis of |
| | | purified poly(A)-tail after |
| | | Ribonuclease T1 digestion |
| Higher order | Spectroscopic analysis to confirm | Circular dichroism (CD) |
| structure (HOS) | the presence and fingerprint of | spectroscopy |
| | HOS | |

### Example 9: Exemplary Specifications for an RNA Drug Substance

The present example describes exemplary specifications for an RNA drug substance manufactured by an in vitro transcription processed as described herein.

TABLE 9-1
Exemplary specifications for an RNA drug substance

| | | |
|---|---|---|
| Quality Attribute | Analytical Procedure | Acceptance Criteria |

Composition and Strength

| Clarity | Appearance (Clarity) a | ≤6 | NTU |
|---|---|---|---|
| Coloration | Appearance (Coloration) a | Not more intensely | |
| | | coloured than level 7 of the | |
| | | brown (B) colour standard | |
| pH | Potentiometry a | 7.0 ± 0.5 | |
| Content (RNA | UV Spectroscopy | 2.25 ± 0.25 | mg/mL |

Identity

| | | |
|---|---|---|
| Identity of Encoded RNA | RT-PCRb | Identity confirmed |
| Sequence | | |

Purity

| | | |
|---|---|---|
| RNA Integrity | Capillary Gel | ≥50% intact RNA |
| | Electrophoresis | |
| 5′- Cap | RP-HPLC | ≥50% |
| Poly(A) Tail | ddPCR | ≥70% |

Process related impurities

| | | |
|---|---|---|
| Residual DNA Template | qPCRb | ≤1000 ng DNA/mg RNA |

Product related impurities

| | | |
|---|---|---|
| dsRNA | Immunoblotb | ≤2000 pg dsRNA/μg RNA |

Safety

| | | | |
|---|---|---|---|
| Bacterial Endotoxin | Endotoxin (LAL) | ≤12.5 | EU/mL |

| | | |
|---|---|---|
| Bioburden | Bioburden | ≤1 CFU/10 mL |

Thus, in some embodiments, as described elsewhere herein, release and/or testing assessments establish that a provided RNA composition is characterized by one or more of:
a) a percentage of capped RNA within a range of about 40-70% or higher, in some embodiments above about 40%, about 50%, about 60%, about 70%, about 80%, about 90%, or more.
b) RNA integrity above about 40%, about 45%, about 50%, about 55%, about 60%, about 65%, about 70%, about 75%, about 80%, about 85%, about 90%, or about 95%.
c) residual dsRNA level below about 2000 pg dsRNA/μg RNA, about 1500 pg dsRNA/μg RNA, about 1000 pg dsRNA/μg RNA, about 500 pg dsRNA/μg RNA, about 250 pg dsRNA/μg RNA, about 100 pg dsRNA/μg RNA, or lower.
d) residual DNA levels within about 0.1-1000 ng DNA per mg RNA, about 50-1,000 ng DNA per mg RNA, about 50-950 ng DNA per mg RNA, about 50-900 ng DNA per mg RNA, about 50-850 ng DNA per mg RNA, or in some embodiments less than or equal to about 500 ng DNA per mg RNA, about 480 ng DNA per mg RNA, about 450 ng DNA per mg RNA, about 420 ng DNA per mg RNA, about 390 ng DNA per mg RNA, about 360 ng DNA per mg RNA, about 330 ng DNA per mg RNA, about 300 ng DNA per mg RNA, about 270 ng DNA per mg RNA, about 240 ng DNA per mg RNA, about 210 ng DNA per mg RNA, or lower.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A method of producing an RNA comprising transcribing RNA from a DNA template using a reaction mix which comprises adenosine triphosphate (ATP), guanosine triphosphate (GTP), cytidine triphosphate (CTP) and uridine triphosphate (UTP), or functional analogs thereof, wherein the starting concentration of UTP, or a functional analog thereof, is lower than the starting concentration of CTP and/or ATP, or a functional analog thereof, wherein the method comprises supplementing the reaction mix during the course of the transcription reaction with a composition which comprises UTP, or a functional analog thereof, and is substantially free of CTP or ATP, or a functional analog thereof.

2. A method of producing an RNA comprising transcribing RNA from a DNA template using a reaction mix which comprises adenosine triphosphate (ATP), guanosine triphosphate (GTP), cytidine triphosphate (CTP) and uridine triphosphate (UTP), or functional analogs thereof, wherein the starting concentration of CTP, or a functional analog thereof, is equal to the starting concentration of ATP, or a functional analog thereof, and wherein the starting concentration of UTP, or a functional analog thereof, is lower than the starting concentration of CTP or ATP, or a functional analog thereof, wherein the method comprises supplementing the reaction mix with UTP, or a functional analog thereof, during the course of the transcription reaction.

3. A method of producing a composition comprising RNA having a reduced double-stranded (ds) RNA content, wherein the method comprises transcribing RNA from a DNA template using a reaction mix which comprises adenosine triphosphate (ATP), guanosine triphosphate (GTP), cytidine triphosphate (CTP) and uridine triphosphate (UTP), or functional analogs thereof, wherein the starting concentration of UTP, or a functional analog thereof, is lower than the starting concentration of CTP and/or ATP, or a functional analog thereof, wherein the method comprises supplementing the reaction mix during the course of the transcription reaction with a composition which comprises UTP, or a functional analog thereof, and is substantially free of CTP or ATP, or a functional analog thereof.

4. A method of producing a composition comprising RNA having a reduced double-stranded (ds) RNA content, wherein the method comprises transcribing RNA from a DNA template using a reaction mix which comprises adenosine triphosphate (ATP), guanosine triphosphate (GTP), cytidine triphosphate (CTP) and uridine triphosphate (UTP), or functional analogs thereof, wherein the starting concentration of CTP, or a functional analog thereof, is equal to the starting concentration of ATP, or a functional analog thereof, and wherein the starting concentration of UTP, or a functional analog thereof, is lower than the starting concentration of CTP or ATP, or a functional analog thereof, wherein the method comprises supplementing the reaction mix with UTP, or a functional analog thereof, during the course of the transcription reaction.

5. The method of claim 3 or 4, wherein the double-stranded (ds) RNA content of the composition comprising RNA is reduced compared to the dsRNA content of a composition comprising RNA transcribed from the same DNA template using equimolar amounts of adenosine triphosphate (ATP), guanosine triphosphate (GTP), cytidine triphosphate (CTP) and uridine triphosphate (UTP), or functional analogs thereof.

6. The method of any one of claims 3 to 5, wherein the immunogenicity of the composition comprising RNA is reduced compared to the immunogenicity of a composition comprising RNA transcribed from the same DNA template using equimolar amounts of adenosine triphosphate (ATP), guanosine triphosphate (GTP), cytidine triphosphate (CTP) and uridine triphosphate (UTP), or functional analogs thereof.

7. The method of any one of claims 1 to 6, wherein uridine triphosphate (UTP), or a functional analog thereof, is present in a starting concentration that limits the rate of transcription.

8. The method of any one of claims 1 to 7, wherein the ratio of the starting concentration of uridine triphosphate (UTP), or a functional analog thereof, to the starting concentration of cytidine triphosphate (CTP) or adenosine triphosphate (ATP), or a functional analog thereof, is between about 1:1.5 and about 1:15.

9. The method of any one of claims 1 to 8, wherein the reaction mix is supplemented with uridine triphosphate (UTP), or a functional analog thereof, when the concentration of UTP, or a functional analog thereof, nears depletion.

10. The method of any one of claims 1 to 9, wherein the reaction mix is supplemented at least once with uridine triphosphate (UTP), or a functional analog thereof, during the course of the transcription reaction.

11. The method of any one of claims 1 to 10, wherein the reaction mix is supplemented continuously with uridine triphosphate (UTP), or a functional analog thereof, during the course of the transcription reaction.

12. The method of any one of claims 1 to 10, wherein the reaction mix is supplemented periodically with uridine triphosphate (UTP), or a functional analog thereof, during the course of the transcription reaction.

13. The method of any one of claims 1 to 12, wherein supplementing the reaction mix with uridine triphosphate (UTP), or a functional analog thereof, maintains or restores the initial ratio of the concentration of UTP, or a functional analog thereof, to the concentration of cytidine triphosphate (CTP) or adenosine triphosphate (ATP), or a functional analog thereof.

14. The method of any one of claims 1 to 13, wherein the reaction mix is supplemented with uridine triphosphate (UTP), or a functional analog thereof, until the end of the transcription reaction.

15. The method of any one of claims 1 to 14, wherein the starting concentration of guanosine triphosphate (GTP), or a functional analog thereof, is lower than the starting concentration of cytidine triphosphate (CTP) or adenosine triphosphate (ATP), or a functional analog thereof.

16. The method of claim 15, wherein guanosine triphosphate (GTP), or a functional analog thereof, is present in a starting concentration that limits the rate of transcription.

17. The method of claim 15 or 16, wherein the ratio of the starting concentration of guanosine triphosphate (GTP), or a functional analog thereof, to the starting concentration of cytidine triphosphate (CTP) or adenosine triphosphate (ATP), or a functional analog thereof, is between about 1:1.5 and about 1:15.

18. The method of any one of claims 15 to 17, wherein the reaction mix is supplemented with guanosine triphosphate (GTP), or a functional analog thereof, during the course of the transcription reaction.

19. The method of claim 18, wherein the reaction mix is supplemented with guanosine triphosphate (GTP), or a functional analog thereof, when the concentration of GTP, or a functional analog thereof, nears depletion.

20. The method of any one of claims 15 to 19, wherein the reaction mix is supplemented at least once with guanosine triphosphate (GTP), or a functional analog thereof, during the course of the transcription reaction.

21. The method of any one of claims 15 to 20, wherein the reaction mix is supplemented continuously with guanosine triphosphate (GTP), or a functional analog thereof, during the course of the transcription reaction.

22. The method of any one of claims 15 to 20, wherein the reaction mix is supplemented periodically with guanosine triphosphate (GTP), or a functional analog thereof, during the course of the transcription reaction.

23. The method of any one of claims 15 to 22, wherein supplementing the reaction mix with guanosine triphosphate (GTP), or a functional analog thereof, maintains or restores the initial ratio of the concentration of GTP, or a functional analog thereof, to the concentration of cytidine triphosphate (CTP) or adenosine triphosphate (ATP), or a functional analog thereof.

24. The method of any one of claims 15 to 23, wherein the reaction mix is supplemented with guanosine triphosphate (GTP), or a functional analog thereof, until the end of the transcription reaction.

25. The method of any one of claims 1 to 24, wherein the method does not comprise supplementing the transcription mix with cytidine triphosphate (CTP) and/or adenosine triphosphate (ATP), or a functional analog thereof, during the course of the transcription reaction.

26. The method of any one of claims 1 to 25, wherein the reaction mix comprises a start nucleotide corresponding to the first nucleotide in the RNA molecule.

27. The method of claim 26, wherein the start nucleotide is a nucleoside monophosphate, a nucleoside diphosphate, a nucleoside triphosphate or a dinucleoside triphosphate.

28. The method of claim 26 or 27, wherein the start nucleotide is a 5′ cap or a 5′ cap analog.

29. The method of claim 28, wherein the 5′ cap analog is selected from the group consisting of G[5′]ppp[5′]G, m⁷G[5′]ppp[5′]G, m₃^{2,2,7}G[5′]ppp[5′]G, m₂^{7,3′-O}G[5′]ppp[5′]G (3′-ARCA), m₂^{7,2′-O}GpppG (2′-ARCA), m₂^{7,2′-O}GppspG D1 (β-S-ARCA D1), m₂^{7,2′-O}GppspG D2 (β-S-ARCA D2) and m₂^{7,3′-O}Gppp(m^{2′-O})ApG (CC413).

30. The method of claim 28 or 29, wherein the 5′ cap or 5′ cap analog in the reaction mix is present in excess compared to guanosine triphosphate (GTP), or a functional analog thereof.

31. The method of claim 30, wherein the ratio of the starting concentration of 5′ cap or 5′ cap analog to the starting concentration of guanosine triphosphate (GTP), or a functional analog thereof, is between about 2:1 and about 20:1.

32. The method of claim 31, wherein the ratio of the starting concentration of 5′ cap or 5′ cap analog to the starting concentration of guanosine triphosphate (GTP), or a functional analog thereof, is about 4:1.

33. The method of any one of claims 1 to 32, wherein the reaction mix further comprises an RNA polymerase, a buffer and at least one monovalent or divalent cation.

34. The method of claim 33, wherein the cation is Li⁺, Na⁺, K⁺, NH₄⁺, tris(hydroxymethyl)aminomethane cation, Mg²⁺, Ba²⁺ or Mn²⁺.

35. The method of claim 33 or 34, wherein the RNA polymerase is selected from the group consisting of T7 RNA polymerase, T3 RNA polymerase and SP6 RNA polymerase.

36. The method of any one of claims 1 to 35, wherein the functional analog of uridine triphosphate (UTP) is selected from the group consisting of Pseudo-UTP, N1-Methylpseudo-UTP, 2-Thio-UTP and 4-Thio-UTP.

37. The method of any one of claims 1 to 36, wherein the functional analog of guanosine triphosphate (GTP) is selected from the group consisting of 7-Deaza-GTP, N1-Methyl-GTP and O6-Methyl-GTP.

38. The method of any one of claims 1 to 37, wherein the DNA template encodes one or more of a 5′ untranslated region (UTR), a 3′ UTR, an open reading frame and a poly(A)-tail.

39. The method of any one of claims 1 to 38, wherein the RNA comprises one or more of a 5′ untranslated region (UTR), a 3′ UTR, an open reading frame and a poly(A)-tail.

40. The method of claim 39, wherein the RNA encodes at least one peptide or protein.

41. The method of any one of claims 1 to 40, wherein the RNA is mRNA.

42. The method of any one of claims 1 to 41, wherein the pH value of the reaction mix is kept substantially constant during the course of the transcription reaction.

43. The method of any one of claims 1 to 42, wherein the progress of the transcription reaction is monitored in real time.

44. The method of any one of claims 1 to 43, wherein the method is performed using a bioreactor.

45. An RNA produced by the method of any one of claims 1 to 44.

46. A composition comprising RNA produced by the method of any one of claims 3 to 44.

47. A method of treating a subject comprising the steps of:
(i) obtaining RNA produced by the method of any one of claims 1 to 44, or obtaining a composition comprising RNA produced by the method of any one of claims 3 to 44, and
(ii) administering the RNA or the composition comprising RNA to the subject.

48. A method of treating a subject by administering the RNA of claim 45 or the composition comprising RNA of claim 46 to the subject.

49. A method of producing an RNA by in vitro transcription, the method comprising:
restricting concentration of UTP or functional analogs thereof during the in vitro transcription reaction.

50. An in vitro transcription reaction comprising:
an RNA template comprising a promoter that directs transcription of a template to generate a transcript with a polyA sequence element;
an RNA polymerase that acts on the promoter; and
adenosine triphosphate (ATP), guanosine triphosphate (GTP), cytidine triphosphate (CTP) and uridine triphosphate (UTP), or functional analogs thereof, wherein the starting concentration of UTP or functional analog thereof, is lower than the concentration of CTP and/or ATP or functional analogs thereof.
